SEQUENCE LISTING (5 sequences)

SEQ ID NO: 1 - DNA, 1857 bp, Homo sapiens
  misc_feature (1)..(1857): n is any nucleic acid
atggcgcagctagagggttactgtttctcggccgccttgagctgtacctttttagtgtcctgcctcctcttctccgccttcagccgggcgctgcgagagccctacatggacgagatcttccacctgcctcaggcgcagcgctactgtgagggccatttctccctttcccagtgggatcccatgattactacattacctggcttgtacctggtgtcagttggagtggtcaaacctgccatttggatctttggatggtctgaacatgttgtctgctccattgggatgctcagatttgttaatcttctcttcagtgttggcaacttctatttactatatttgcttttccacaaggtacaacccagaaacaaggctgcctcaagtatccagagagtcttgtcaacattaacactagcagtatttccaacactttatttttttaacttcctttattatacagaagcaggatctatgttttttactctttttgcatatttgatgtgtctttatggaaatcataaaacttcagccttccttggattttgtggcttcatgtttcggcaaacaaatatcatctgggctgtcttctgtgcagggaatgtcattgcacaaaagttaactgaggcttggaaaactgagctacaaaagaaggaagacagacttccacctattaaaggaccatttgcagaattcagaaaaattcttcagtttcttttggcttattccatgtcctttaaaaacttgagtatgcttttctgtttgacttggccctacatccttctgggatttctgttttgtgcttttgtagtagttaatggtggaattgttattggcgatcggagtagtcatgaagcctgtcttcattttcctcaactattctactttttttcatttactctctttttttcttttcctcatctcctgtctcctagcaaaattaagacttttctttccttagtttggaaacatggaattctgtttttggtggttaccttagtctctgtgtttttagtttggaaattcacttatgctcataaatacttgctagcagacaatagacattatactttctatgtgtggaaaagagtttttcaaagatatgcaattctgaaatatttgttagttccagcctatatatttgctggttggagtatagctgactcattgaaatcaaagccaattttttggaatttaatgtttttcatatgcttgttcattgttatagttcctcagaaactgctggaatttcgttacttcattttaccttatgtcatttataggcttaacataactctgcctcccacatccagacttgtttgtgaactgagttgctatgcaattgttaatttcataactttttacatctttctgaacaagacttttcagtggccaaatagtcaggacattcaaaggtttatgtggtaatatcagtgatattttgaactgtaaaaatggacttaataatagaccatttctacaaagaacaactgaataggnggaaaacatggaatttcttttaggtgcagtggtggtcttcaaattacattagtttttttaatatatattttaaacatatgtaagaaattaagtggcaaagaactgggaaagcttaagacctgcttcaaangcctgaataatgggaaaataaanwngtttncagatatctcatatcgctcnnnknatgntggcccytmncaangcttgggaatgkttnnwntgnataagttnattaaanctgggnntgctnnmwatnacttnnnknccanccwnnnwacnatgnnntannnantatttacaaaggtcaggtgatattcttgactgaaaagtgctctnaacataaaagtaaatatgngccncaaa

SEQ ID NO: 2 - protein, 473 aa, Homo sapiens
  misc_feature (1)..(1857): n is any nucleic acid
MAQLEGYCFSAALSCTFLVSCLLFSAFSRALREPYMDEIFHLPQAQRYCEGHFSLSQWDPMITTLPGLYLVSVGVVKPAIWIFGWSEHVVCSIGMLRFVNLLFSVGNFYLLYLLFHKVQPRNKAASSIQRVLSTLTLAVFPTLYFFNFLYYTEAGSMFFTLFAYLMCLYGNHKTSAFLGFCGFMFRQTNIIWAVFCAGNVIAQKLTEAWKTELQKKEDRLPPIKGPFAEFRKILQFLLAYSMSFKNLSMLFCLTWPYILLGFLFCAFVVVNGGIVIGDRSSHEACLHFPQLFYFFSFTLFFSFPHLLSPSKIKTFLSLVWKHGILFLVVTLVSVFLVWKFTYAHKYLLADNRHYTFYVWKRVFQRYAILKYLLVPAYIFAGWSIADSLKSKPIFWNLMFFICLFIVIVPQKLLEFRYFILPYVIYRLNITLPPTSRLVCELSCYAIVNFITFYIFLNKTFQWPNSQDIQRFMW

SEQ ID NO: 3 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 4 - protein, 123 aa, Homo sapiens
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 5 - DNA, 732 bp, Homo sapiens
caaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctctgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaattt

(19) United States
(12) Patent Application Publication
(10) Pub. No.: US 2003/0013136 A1
(43) Pub. Date: Jan. 16, 2003

(54) Human KCR1 regulation of HERG potassium channel block

(76) Inventors: Jeffrey R. Balser, Brentwood, TN (US); Alfred L. George JR., Brentwood, TN (US); Dan M. Roden, Nashville, TN (US)

Correspondence Address: JENKINS & WILSON, PA, 3100 TOWER BLVD, SUITE 1400, DURHAM, NC, 27707, US

(21) Appl. No.: 10/000,151

(22) Filed: Oct. 30, 2001

Related U.S. Application Data
(60) Provisional application No. 60/244,340, filed on Oct. 30, 2000.

(51) Int. Cl. G01N 33/567; C12Q 1/68; C12P 21/02; C12N 5/06; C12N 15/85

(52) U.S. Cl. 435/7.21; 435/6; 435/455; 435/325

(57) ABSTRACT

The present invention discloses methods relating to screening methods and methods of identifying a compound that can modulate HERG potassium channel activity. The methods generally employ at least HERG and KCR1 polypeptides. The disclosed methods can be applied in the development of a candidate pharmaceutical or they can be employed to evaluate presently marketed pharmaceuticals.

### CROSS REFERENCE TO RELATED APPLICATIONS

The present patent application claims benefit of U.S. Provisional Application Serial No. 60/244,340, entitled “Human KCR1 Regulation of HERG Potassium Channel Block”, which was filed Oct. 30, 2000 and is incorporated herein by reference.

### TECHNICAL FIELD

The present invention relates generally to modulation of potassium channels, and more particularly potassium channels encoded by the HERG gene. The present invention also relates to modulation of potassium channels encoded by the HERG gene coexpressed with the protein KCR1.

Abbreviations

| | |
|---|---|
| LOT | long QT |
| CHO | Chinese hamster ovary |
| HERG | human EAG related gene |
| EAG | ether-a-go-go |
| EST | expressed sequence tag |
| FLIPR | fluorometric imaging plate reader |
| cNBD | cyclic nucleotide binding domain |

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| Amino Acid | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### Background Art

Cardiac action potential is repolarized (i.e. terminated) by currents through K+channels, and both acquired and inherited (congenital) arrhythmias can be triggered by drugs and genetic defects that suppress cardiac K⁺ currents, leading to a condition known as “Long QT Syndrome” (Keating & Sanguinetti, (1996) Science 272: 681-685; Splawski et al., (1997) Nat Genet 17: 338-340; and Wang et al., (1996) Nat Genet 12:17-23). The human ether-a-go-go-related gene (HERG) (Warmke & Ganetzky, (1994) Proc Natl Acad Sci USA 91: 3438-3442) encodes the major pore-forming protein (i.e. a “HERG channel”), which is involved in a prominent repolarizing K⁺ current in the heart, known as “I_{Kr}” (Curran et al., (1995) Cell. 80: 795-803). Drug-induced suppression of this pore-forming protein, which can be intentional or a side effect of the drug, can provoke abnormal cardiac repolarization and ventricular arrhythmias, but this effect is often unpredictable (Roden, (1998) Pacing Clin Electrophysiol 21: 1029-1034). This wide variability in clinical response suggests that modulating factors might critically influence HERG block (i.e. Inhibition), both positively and negatively.

HERG ion channels, encoded by the HERG gene, are inwardly rectifying potassium channels. HERG channels have properties consistent with the gating properties of ether-a-go-go (EAG) potassium channels, and other outwardly-rectifying S4-containing potassium channels, but with the addition of an inactivation mechanism that attenuates potassium efflux during depolarization. These properties of HERG channel function are critical to maintaining normal cardiac rhythmicity. The molecular mechanism by which HERG ion channels protect the heart against inappropriate rhythmicity has been elucidated by Smith et al., (Smith et al., (1996) Nature 379: 33) and by Miller (Miller, (1996) Nature 379: 767). The role of HERG channels in long QT syndrome also has been an area of interest, although until the present invention, the precise effects of interactions between HERG channels and other proteins (e.g., KCR1) has not be elucidated.

Acquired LQT usually results from therapy with medications that block cardiac K⁺ channels (Roden, (1988). Arrhythmogenic Potential of Class III Antiarrhythmic Agents: Comparison with Class I Agents. in Control of Cardiac Arrhythmias by Lengthening Repolarization, Singh (ed.). Mt. Kisco, N.Y., Futura Publishing Co., pp. 559-576.), while inherited long QT syndrome is primarily a gene-based condition, although it can be aggravated by certain drugs and medications. The medications most commonly associated with long QT (LQT) are antiarrhythmic drugs (e.g., quinidine, sotalol) that block the cardiac rapidly-activating delayed rectifier K⁺ current, I_{Kr}, as part of their spectrum of pharmacologic activity. Thus, these medications block HERG channels.

Other drugs might also contribute to acquired LQT. These include antihistamines and some antibiotics such as erythromycin. I_{Kr} has been characterized in, among other systems, isolated cardiac myocytes (Balser et al., (1990). J. Gen. Physiol. 96: 835-863; Follmer et al., (1992). Am. J. Physiol. 262: C75-C83; Sanguinetti & Jurkiewicz; (1990) J Gen Physiol 96: 195-215; Shibasaki, (1987). J. Physiol. 387: 227-250; Yang et al., (1994). Circ. Res. 75: 870-878.), and is known to have an important role in initiating repolarization of action potentials. Acquired LQT can cause a range of adverse conditions, including death. Often, the risk of acquired LQT is a risk that must be assumed by a patient taking a medication known to be associated with LQT.

It would be of great significance to be able to design medications that minimize the risk of acquired long QT syndrome. Such medications could be prescribed and employed with the confidence that a patient can take the medication with minimal risk of developing a cardiac arrhythmia or other adverse condition. It would also be advantageous to be able to identify medications that have an ability to contribute to acquired LQT. If medications could be screened for this property prior to administration or even before clinical trials are initiated, many candidate therapeutics could be eliminated from further testing, thus saving a drug developer time and money, as well as reducing the risk of harm to a patient. This and other problems are solved by the present invention.

### SUMMARY OF THE INVENTION

A method of identifying a compound known or suspected to modulate a biological activity of a potassium channel is disclosed. In a preferred embodiment, the method comprises: (a) providing a structure comprising a potassium channel polypeptide and a KCR1 polypeptide; (b) contacting the test compound with the structure; (c) determining a biological activity of the potassium channel polypeptide in the presence of the test compound; (d) comparing the biological activity of the potassium channel polypeptide in the presence of the test compound to the biological activity of the potassium channel polypeptide in an absence of the test compound, wherein a difference between the biological activity of the potassium channel in the absence of the test compound and the biological activity of the potassium channel polypeptide in the presence of test compound indicates modulation of a biological activity of the potassium channel.

Additionally, a method of identifying a candidate compound as a HERG channel inhibitor is disclosed. In a preferred embodiment, the method comprises: (a) providing a structure comprising a HERG potassium channel and a KCR1 polypeptide; (b) contacting a candidate compound with the structure; (c) determining a biological activity of the HERG potassium channel in the presence of the candidate compound; (d) comparing the activity in the presence of the candidate compound with the biological activity of the HERG potassium channel in an absence of the candidate compound; and (e) selecting the candidate compound as a HERG potassium channel inhibitor if the biological activity of the HERG potassium channel in the presence of the candidate compound is lower than the biological activity of the HERG potassium channel in the absence of the candidate compound.

Additionally, a method of predicting a propensity of a candidate drug to induce cardiac arrhythmia is disclosed. In a preferred embodiment, the method comprises: (a) providing a structure comprising a potassium channel and a KCR1 polypeptide; (b) contacting a candidate drug with the structure; (c) determining a biological activity of the potassium channel in the presence of the candidate drug; and (d) comparing the biological activity of the potassium channel in the presence of a KCR1 polypeptide and in an absence of a candidate drug to a biological activity of the potassium channel in the presence of the candidate drug, wherein a biological activity of the potassium channel in the presence of a candidate drug that is less than a biological activity of the potassium channel in an absence of the candidate drug is indicative of a propensity of the drug to induce cardiac arrhythmia.

In each of the foregoing embodiments of a method of the present invention, it is preferred that the potassium channel is HERG. More preferably, the HERG comprises a polypeptide sequence as set forth in SEQ ID NO: 3, even more preferably the HERG is disposed in a cell or a lipid bilayer, and even more preferably the HERG is in an activated state.

In each of the foregoing embodiments of a method of the present invention, it is preferred that the KCR1 is derived from a human and comprises the nucleic acid sequence of SEQ ID NO: 1. Optionally, the cell further comprises a MiRP1 polypeptide. In this case, the MiRP1 polypeptide is preferably encoded by a nucleic acid comprising SEQ ID NO: 4. In each of the foregoing embodiments of the methods of the present invention, it is also preferable that the determining comprises employing a patch clamp apparatus.

A method of identifying a candidate compound that modulates the biological activity of a complex comprising a HERG channel polypeptide and a KCR1 polypeptide is disclosed. In a preferred embodiment, the method comprises: (a) placing a cell comprising a HERG channel polypeptide and a KCR1 polypeptide into a bathing solution; (b) determining an induced K⁺ current in the cell of step (a); (c) adding a candidate drug to the bathing solution of step (a); (d) determining an induced K⁺ current in the cell of step (c); and (e) comparing the induced current of step (b) with the induced current of step (d), wherein the candidate compound modulates the biological activity of a complex comprising a HERG channel polypeptide and a KCR1 polypeptide if the current of step (d) is different from the current of step (b).

Preferably, the HERG channel polypeptide comprises a polypeptide sequence as set forth in SEQ ID NO: 3, even more preferably is disposed in a cell or a lipid bilayer, and even more preferably is in an activated state. Preferably, the KCR1 is derived from a human and more preferably, comprises the nucleic acid sequence of SEQ ID NO: 1. Optionally, the cell further comprises a MiRP1 polypeptide, which is preferably encoded by a nucleic acid comprising SEQ ID NO: 4. It is also preferable that the determining comprises employing a patch clamp apparatus. Optionally, the cell is transfected with a nucleic acid sequence encoding a HERG channel polypeptide and a nucleic acid sequence encoding a KCR1 polypeptide.

A method for identifying a candidate compound as a modulator of KCR1 expression is also disclosed. In one embodiment, the method comprises: (a) contacting a eukaryotic cell sample with a predetermined concentration of the candidate compound to be tested, the cell sample comprising at least one cell comprising a DNA construct comprising in 5′ to 3′ order (i) a modulatable transcriptional regulatory sequence of a KCR1-encoding gene, (ii) a promoter of the KCR1-encoding gene, and (iii) a reporter gene which expresses a polypeptide capable of producing a detectable signal coupled to and under the control of the promoter, under conditions such that the candidate compound if capable of acting as a transcriptional modulator of the gene encoding the protein of interest, causes a measurable detectable signal to be produced by the polypeptide expressed by the reporter gene; (b) quantitatively determining the amount of the signal so produced; and (c) comparing the amount so determined with the amount of produced signal detected in the absence of candidate compound being tested or upon contacting the cell sample with other compounds so as to thereby identify the candidate compound as a chemical which causes a change in the detectable signal produced by the polypeptide and which transcriptionally modulates expression of KCR1.

In another embodiment, the method comprises: (a) contacting a eukaryotic cell sample with a predetermined concentration of the candidate compound to be tested, the cell sample comprising at least one cell comprising a DNA construct comprising in 5′ to 3′ order (i) a modulatable transcriptional regulatory sequence of a KCR1-encoding gene, (ii) a promoter of the KCR1-encoding gene, and (iii) a DNA sequence transcribable into mRNA coupled to and under the control of the promoter, under conditions such that the candidate compound if capable of acting as a transcriptional modulator of the KCR1-encoding gene, causes a measurable difference in the amount of mRNA transcribed from the DNA sequence; (b) quantitatively determining the amount of the mRNA so produced; and (c) comparing the amount so determined with the amount of mRNA detected in the absence of candidate compound being tested or upon contacting the cell sample with other compounds so as to thereby identify the candidate compound as a compound which causes a change in the detectable mRNA amount and which transcriptionally modulates expression of KCR1.

Optionally, each of the foregoing embodiments can further comprise separately contacting each of a plurality of identical cell samples with different candidate compounds, each cell sample containing a predefined number of identical cells under conditions wherein said contacting is effected with a predetermined concentration of each different candidate compound to be tested. Modulators identified by the methods are also provided, as are methods of using the modulators.

A method for modulating potassium channel function in a subject is also provided. The method comprises: (a) administering to the subject an effective amount of a substance that provides elevated expression of a KCR1-encoding nucleic acid molecule in a cell or tissue where modulated potassium channel function is desired; and (b) modulating potassium channel function in the subject through the administering of step (a). In a preferred embodiment, the method comprises: (a) providing a gene therapy construct comprising a nucleotide sequence encoding a KCR1 polypeptide; and (b) administering the gene therapy construct to a subject, whereby the function of a potassium channel in the subject is modulated. More preferably, the potassium channel activity that is altered in the subject comprises HERG activity.

A method of modulating KCR1 expression in a subject in need thereof is also provided. In a preferred embodiment, the method comprises administering to the vertebrate an effective amount of a substance capable of modulating expression of a KCR1-encoding nucleic acid molecule. Optionally, the substance that modulates expression of the KCR1-encoding nucleic acid molecule comprises an antisense oligonucleotide or a ligand for a modulatable transcriptional regulatory sequence of a KCR1-encoding nucleic acid molecule or for a promoter of the KCR1-encoding nucleic acid molecule.

A method of screening for a susceptibility to a drug-induced cardiac arrhythmia in a subject is disclosed. The method comprises: (a) obtaining a biological sample from the subject; and (b) detecting a polymorphism of a KCR1 gene in the biological sample from the subject, the presence of the polymorphism indicating the susceptibility of the subject to a drug-induced cardiac arrhythmia.

Kits and reagents, including oligonucleotides, nucleic acid probes and antibodies suitable for use in carrying out the methods of the present invention and for use in detecting KCR1 polypeptides and polynucleotides are also disclosed herein.

Accordingly, it is an object of the present invention to provide a method of identifying a compound known or suspected to modulate a biological activity of a potassium channel. This and other objects are achieved in whole or in part by the present invention.

An object of the invention having been stated hereinabove, other objects will be evident as the description proceeds, when taken in connection with the accompanying Examples and Drawings as best described hereinbelow.

### BRIEF DESCRIPTION OF THE DRAWINGS

FIG. 1A is an alignment depicting the alignment of deduced amino acid sequences of the rat and human KCR1, with identical amino acids in the human sequence identified by the dashes. Putative transmembrane segments (TMD 1, 2, 3, 4, 5, 6, 7, 8, 9, 10, 11 and 12) were identified by hydropathy analysis.

FIG. 1B is a Northern blot analysis of 2 μgs of poly A+RNA on a Human Multiple Tissue Northern Blot (Clontech of Palo Alto, Calif.). Lane 1: heart; lane 2: brain; lane 3: placenta; lane 4: lung; lane 5: liver; lane 6: skeletal muscle; lane 7: kidney; lane 8: pancreas; RNA size markers are indicated in kb.

FIG. 2A is a current trace depicting time-dependent HERG block by dofetilide. The voltage-clamp protocol is shown at the top of the figure. Depicted are currents recorded during a pre-drug (control) period, and following a 4 min exposure to 300 nM dofetilide. For HERG alone, the depolarization-induced current exhibits a time-dependent decline with drug exposure (Snyders & Chaudhary; (1996) Mol Pharmacol 49: 949-955). This time-dependent blocking effect is markedly attenuated by KCR1 coexpression.

FIG. 2B is a current trace depicting time-dependent HERG+KCR1 block by dofetilide inhibited by KCR1. The voltage-clamp protocol is shown at the top of the figure. Depicted are currents recorded during a pre-drug (control) period, and following a 4 min exposure to 300 nM dofetilide. For HERG alone, the depolarization-induced current exhibits a time-dependent decline with drug exposure (Snyders & Chaudhary; (1996) Mol Pharmacol 49: 949-955). This time-dependent blocking effect is markedly attenuated by KCR1 coexpression.

FIG. 3A is a plot depicting pulse-dependent block with 20 nM dofetilide. Currents were recorded from cells expressing HERG alone. Depolarizing pulses (shown at the top of FIG. 1A) were applied every 10 seconds, and the amplitude of the tail current at −50 mV is plotted relative to the size of the tail current measured prior to drug exposure. After 20 minutes in 20 nM dofetilide (solid circles), tail currents recorded from HERG alone (mean±S.E. n=7) were suppressed more than those recorded from HERG+KCR1 (n=12). With d-sotalol perfusion (solid triangles), tail currents recorded in HERG alone (n=3) were also suppressed more than those of HERG+KCR1 (n=7). The currents recorded in the drug-free bath solution over the same time period (open squares) were not altered for either HERG (n=4) or HERG+KCR1 (n=5).

FIG. 3B is a plot depicting is inhibition of pulse-dependent block with 20 nM dofetilide by KCR1. Currents were recorded from cells expressing HERG+KCR1. Depolarizing pulses (shown at the top of FIG. 1A) were applied every 10 seconds, and the amplitude of the tail current at −50 mV is plotted relative to the size of the tail current measured prior to drug exposure. After 20 minutes in 20 nM dofetilide (solid circles), tail currents recorded from HERG alone (mean±S.E., n=7) were suppressed more than those recorded from HERG+KCR1 (n=12). With d-sotalol perfusion (solid triangles), tail currents recorded in HERG alone (n=3) were also suppressed more than those of HERG+KCR1 (n=7). The currents recorded in the drug-free bath solution over the same time period (open squares) were not altered for either HERG (n=4) or HERG+KCR1 (n=5).

FIG. 3C is a plot depicting the concentration dependence of block by dofetilide in HERG alone and HERG+KCR1. Mean data were fitted to a logistic expression (1/1+{[D]/IC₅₀}ⁿ), where [D] is the dofetilide concentration and n is the Hill coefficient. For HERG alone, the IC₅₀ for dofetilide block was 15.2 nM (n=0.99), while for HERG+KCR1, the IC₅₀ was 59.7 nM (n=0.96). Values in parentheses indicate the number of cells at each drug concentration.

FIG. 3D is a plot depicting the effect of KCR1 on quinidine block. The figure demonstrates that quinidine block developed very rapidly, reaching equilibrium within the first few test pulses, however, block of HERG alone (n=5) was greater than that of HERG+KCR1 (n=6).

FIG. 4A depicts the effects of KCR1 on the gating properties of HERG channels expressed in mammalian cells. Representative families of current traces were recorded from HERG. The voltage clamp protocol is shown (at the top of the figure). Cells were held at −80 m V, and then stepped to test potentials between +70 and −70 mV in 10 mV increments for 2 seconds before repolarizing to −50 mV.

FIG. 4B depicts the effects of KCR1 on the gating properties of HERG channels expressed in mammalian cells. Representative families of current traces were recorded from HERG+KCR1. The voltage clamp protocol is the same as that of FIG. 1A. Cells were held at −80 m V, and then stepped to test potentials between +70 and −70 mV in 10 mV increments for 2 seconds before repolarizing to −50 mV.

FIG. 4C depicts the effects of KCR1 on the gating properties of HERG channels expressed in mammalian cells. Representative families of current traces were recorded from HERG+KCR1. Filled symbols indicate HERG currents recorded at the end of each 2 sec depolarizing pulse (denoted by a solid arrow in FIG. 4A), and are plotted as the current relative to that recorded at +20 mV in the same cell. The open symbols indicate peak outward tail currents measured upon repolarization at −50 mV (denoted by a dashed arrow in FIG. 4A), and represent the voltage-dependence of activation. These were normalized to the maximum current recorded in the same cell, and were then fitted to a Boltzmann function (solid lines) of the form: I=1/[1+exp(Vₜ−V_{1/2})/δ)], where V_{1/2} is the midpoint of activation and δ is a slope factor There was no significant difference in the current-voltage relationship between HERG (n=42) and HERG+KCR1 (n=43).

FIG. 4D depicts the voltage-dependent distribution between the open and inactivated states for HERG alone (n=19, solid 20 squares) and HERG+KCR1 (n=20, open squares). Measurements were made using the voltage-clamp protocol shown (inset), and a representative current trace is also shown. Following activation, the membrane potential was stepped to the test potentials from +60 to −130 mV for 12.5 ms to allow channels to recover from inactivation, and then current tails at +30 mV were measured and plotted in panel d as a function of the test potential. KCR1 did not affect HERG voltage dependence of inactivation.

FIG. 5A is a plot depicting the observation that MiRP1 and KCR1 have antagonistic effects on dofetilide block. In FIG. 5A, the voltage-clamp protocol and drug exposure were the same as in FIGS. 3A and 3B, except the dofetilide concentration was 100 nM. Cells expressing HERG (n=5), HERG+KCR1 (n=5), HERG+MiRP1 (n=4) and HERG+KCR1+MiRP1 (n=5g) were compared. Dofetilide block was markedly reduced by KCR1, but this effect was attenuated by MiRP1 coexpression.

FIG. 5B is a current trace confirming coexpression of MiRP1 by measuring the rate of current decay at −120 mV following a 2 second depolarization to +20 mV, and was performed prior to drug treatment. Shown superimposed are representative decaying currents from cells expressing HERG alone, HERG+MiRP1, or HERG+MiRP1+KCR1. MiRP1 increased the deactivation rate of HERG, as shown previously, and KCR1 counteracted this effect of MiRP1.

FIG. 5C is a bar graph confirming interaction between HERG, MiRP1, and KCR1. To generate this graph, deactivating current tails at −120 mV were fitted to a double exponential of the form y=A₁*e^{-(t-t0)/Tfast}+A₂*e^{-(t-t0)/Tslow}. The bar graph shows the fast and slow time constants obtained from fitting current recorded in individual cells. The fast time constants for deactivation of HERG, HERG+KCR1, HERG+MiRP1 and HERG+KCR1+MiRP1 were 22.5±1.3 sec (n=9), 22.6±1.7 sec (n=12), 18.3±0.8 sec (n=13) and 22.5±1.3 sec (n=17), respectively. The slow time constants of HERG, HERG+KCR1, HERG+MiRP1 and HERG+KCR1+MiRP1 were 263±12 sec (n=9), 230±19 sec (n=12), 199±10 sec (n=13) and 251±13 sec (n=17), respectively. Both the fast and slow time constants of HERG+MiRP1 deactivation were substantially reduced compared to HERG alone (p<0.05), and this gating effect was inhibited by KCR1.

### DETAILED DESCRIPTION OF THE INVENTION

In one aspect, the present invention addresses interactions between HERG and KCR1. These proteins have been implicated in Long QT Syndrome, which arises from the intentional or inadvertent blocking of HERG potassium channels. In another aspect of the invention, the observation that the blocking effects of a HERG inhibitor are attenuated by the presence of KCR1 is disclosed. Additionally, the present invention discloses the observation that the blocking effects of a HERG inhibitor are augmented by the presence of KCR1 and MiRP1.

Thus, the present invention discloses methods of identifying a compound known or suspected of modulating a biological activity of a potassium channel, identifying a candidate compound as a HERG channel inhibitor and methods of identifying a candidate compound as a HERG channel inhibitor. Methods of predicting a propensity of a candidate drug to induce a cardiac arrhythmia and methods of identifying a drug that modulates HERG and/or KCR1 activity are also disclosed. Additionally, a method of modulating potassium channel blocking is also provided in accordance with the present invention.

The methods of the present invention provide for the rapid identification of candidate therapeutics that pose a potential risk for inducing long QT syndrome. Following identification, such therapeutics can be redesigned or even removed from a research program, thereby preventing accidental injury to, and/or death of, a subject. Therapeutics can be screened, for example, based on their observed interactions with a HERG channel and/or the combination of a HERG channel and a KCR1 polypeptide (and/or a MiRP1 polypeptide). These and other goals can be achieved by employing the present invention. Detailed descriptions of these and other applications follow hereinbelow.

I. Definitions

Following long-standing patent law convention, the terms “a” and “an” mean “one or more” when used in this application, including the claims.

As used herein, the term “host cell” means a cell into which a heterologous nucleic acid molecule has been introduced. Any suitable host cell can be used, including but not limited to eukaryotic hosts such as mammalian cells (e.g., CHO cells, tsA201 cells, HEK-293 cell, HeLa cells, CV-1 cells, COS cells), amphibian cells (e.g., Xenopus oocytes), insect cells (e.g., Sf9 cells), as well as prokaryotic hosts such as E. coli and Bacillus subtilis. A preferred host cell comprises a cell substantially lacking a HERG channel polypeptide and/or a KCR1 polypeptide. Preferred host cells also include, but are not limited to, mammalian cells, and are more preferably human cells.

As used herein, the term “determine” and grammatical derivatives thereof mean qualitative and/or quantitative determinations, including measuring current, voltage, and the like.

As used herein, the term “expression,” and grammatical derivatives thereof, generally refers to the cellular processes by which a polypeptide is produced from RNA. The term “coexpression” and grammatical derivatives thereof generally refers to the cellular processes by which two or more polypeptides are produced from RNA.

As used herein, the term “biological activity” means any observable effect flowing from HERG channel operation. Representative, but non-limiting, examples of biological activity in the context of the present invention include transmission of potassium ions through a HERG channel.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product. Preferably, a polypeptide encompasses a stretch of amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in preferred embodiments, at least about 30 or more amino acids, e.g., 35, 40, 45, 50, 60, 70, etc.

As used herein, the term “modulate” means an increase, decrease, or other alteration of any, or all, chemical and biological activities or properties of a HERG polypeptide and/or KCR1 polypeptide. The term “modulation” as used herein refers to both upregulation (i.e., activation or stimulation) and downregulation (i.e. inhibition or suppression) of a response.

As used herein, the terms “nucleic acid sequence encoding a HERG polypeptide,” “nucleic acid sequence encoding a KCR1 polypeptide,” and “nucleic acid sequence encoding a MiRP1 polypeptide” can refer to one or more coding sequences within a particular individual. Preferably, a “nucleic acid sequence encoding a HERG polypeptide” comprises a nucleotide sequence encoding a polypeptide as set forth in SEQ ID NO: 3. Preferably, a “nucleic acid sequence encoding a KCR1 polypeptide” comprises a human KCR1 nucleic acid sequence, and more preferably comprises a nucleic acid sequence comprising SEQ ID NO: 1. Preferably, a “nucleic acid sequence encoding a MiRP1 polypeptide” comprises a nucleic acid sequence comprising SEQ ID NO: 4. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity. The evaluation of allelic differences and identification and characterization of polymorphisms are also disclosed herein.

As used herein, the term “cell” means not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “HERG,” “HERG polypeptide” and “HERG channel” are used interchangeably and in a preferred embodiment mean a polypeptide comprising a polypeptide sequence as set forth in SEQ ID NO: 3 and biological equivalents thereof. A “HERG polypeptide” preferably exhibits the ability to transport potassium ions. However, the present invention provides mutations in the sequence of SEQ ID NO: 3, which might lead to a HERG polypeptide that is incapable of transporting potassium ions, or which transports potassium ions at a higher or lower rate than a wild-type HERG polypeptide; such a HERG mutant still falls under the definition of the term “HERG polypeptide.” A “HERG polypeptide” can comprise greater or fewer number of amino acids than those disclosed in SEQ ID NO: 3.

As used herein, the term “KCR1 polypeptide” and “KCR1” are used are used interchangeably and in a preferred embodiment mean a polypeptide encoded by a human KCR1 nucleic acid sequence, and more preferably by a nucleic acid sequence comprising SEQ ID NO: 1, and biological equivalents thereof. A “KCR1 polypeptide” preferably exhibits the ability to attenuate blocking of a HERG channel by a drug. However, the present invention provides mutations in the sequence of SEQ ID NOs: 1 and 2, and methods for detecting the same, which might lead to a KCR1 polypeptide that is incapable of attenuating blocking of a HERG channel by a drug, or which attenuates blocking of a HERG channel by a drug to a higher or lower degree than a wild-type KCR1 polypeptide; such a KCR1 mutant still falls under the definition of the term “KCR1 polypeptide”. A “KCR1 polypeptide” can comprise greater or fewer number of nucleotides and/or amino acids than those disclosed in SEQ ID NOs: 1 and 2.

As used herein, the term “MiRP1 polypeptide” and “MiRP1” are used are used interchangeably and in a preferred embodiment mean a polypeptide encoded by a nucleic acid sequence comprising SEQ ID NO: 4 and biological equivalents thereof. A “MiRP1 polypeptide” preferably exhibits the ability to augment blocking of a HERG channel by a drug in the presence of KCR1. However, the present invention provides mutations in the sequence of SEQ ID NOs: 4 and 5, which might lead to a MiRP1 polypeptide that is incapable of augmenting blocking of a HERG channel by a drug in the presence of KCR1, or which augments blocking of a HERG channel by a drug in the presence of KCR1 to a higher or lower degree than a wild-type MiRP1 polypeptide; such a MiRP1 mutant still falls under the definition of the term “MiRP1 polypeptide.”A “MiRP1 polypeptide” can comprise greater or fewer number of nucleotides and/or amino acids than those disclosed in SEQ ID NOs: 4 and 5.

As used herein, the term “mutation,” and grammatical derivations thereof, carries its traditional connotation and means a change, inherited, naturally occurring or introduced, in a nucleic acid or polypeptide sequence, and is used in its sense as generally known to those of skill in the art.

As used herein, the term “potassium channel” means any structure, including particularly a polypeptide, adapted to transmit potassium ions. A protein encoded by the HERG gene is a preferred potassium channel.

As used herein, the term “lipid bilayer” means any structure comprising two layers of phospholipids that are oriented lipid-to-lipid. A lipid bilayer can form a membrane of a cell or it can exist ex vivo. When a lipid bilayer exists ex vivo, it can exist, for example, on a glass or plastic plate, which can also serve as a frame for the lipid bilayer. Lipid bilayers can also be isolated from an organism, such as a prokaryote.

As used herein, the terms “patient” and “subject” are used interchangeably and generally encompass any individual that is at risk for developing an adverse effect associated with exposure to a medication. The terms refer to any organism that has taken (or to which has been administered) or are contemplating taking (or to which administration has been contemplated) a given drug or medication. As used herein, the “patient” and “subject” need not refer exclusively to human beings, which is preferred, but can also refer to animals such as mice, rats, dogs, poultry, and Drosophila and even individual cells, such as Chinese hamster ovary (CHO) cells. The methods of the present invention are particularly useful in the treatment and diagnosis of warm-blooded vertebrates. Thus, the invention concerns mammals and birds. More particularly, provided is the treatment and/or diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

As used herein, the terms “potassium channel block” and “block,” as well as grammatical derivatives thereof, mean an inhibition of a potassium channel. The terms specifically encompass a potassium channel that is maintained in a conformation facilitating the continuous or intermittent transmission of potassium ions through the channel.

As used herein, the term “bathing solution” means a solution in which one or more cells can be maintained in a viable state. The term also encompasses a solution in which a lipid bilayer can be maintained. Thus, a bathing solution preferably comprises salts and nutrients to maintain the cell, as well as to maintain a desired pH and tonicity. A bathing solution can comprise, for example, 145 mM NaCl; 4 mM KCl; 1.8 mM CaCl₂; 1.0 mM MgCl₂; 10 mM HEPES; and 10 mM glucose, which is maintained at 22-25 degrees C. and pH 7.35.

As used herein, the term “long QT syndrome” means a type of ventricular tachycardia that is commonly associated with excessive prolongation of the electrocardigraphic QT interval. The terms encompass both acquired long QT syndrome as well as inherited long QT syndrome. Long QT syndrome is typically associated with the presence of torsades de pointes.

As used herein, the term “exogenous” means originating, produced or manufactured outside of a subject body, cell, or organ. For example, an exogenous nucleic acid sequence can be produced outside of a cell or organism and subsequently transfected into the cell. The term “exogenous” is not species dependent and can refer to nucleic acids originating outside a given cell or organism and in a species different from the given cell or organism.

As used herein, the terms “drug,” “agent”, “candidate compound”, “compound”, “small molecule”, and “medication” are used interchangeably and mean a chemical entity intended to effectuate a change in an organism or model system. Preferably, but not necessarily, the organism is a human being. It is not necessary that a drug be known to effectuate a change in an organism; chemical entities that are suspected, predicted or designed to effectuate a change in an organism are therefore encompassed by the term “drug.” The effectuated change can be of any kind, observable or unobservable, and can include, for example, a change in the biological activity of a protein. These terms specifically encompass an agent that is being screened for its effect on HERG and/or KCR1 biological activity.

II. General Considerations

Screening for HERG blockade is a primary concern of the pharmaceutical industry, since compounds that block HERG channels in heterologous expression systems usually also suppress the I_{Kr} potassium current in the heart. This can threaten the well being of a patient. I_{Kr} suppression by drugs causes the acquired long QT syndrome that evokes idiopathic ventricular arrhythmias and sudden death in 1-8% of patients taking such agents. The present invention shows that a human gene product, hKCR1, attenuates block of HERG channels by at least 3 compounds (d-sotalol, dofetalide, quinidine) that normally have high affinity (in the nanomolar range) for HERG.

The data and studies presented in the Laboratory Examples were conducted in a heterologous expression system (Chinese hamster ovary cells, CHO-K1) where whole-cell HERG currents were measured (and drug block was assessed) by employing voltage-clamp methods. This approach is sensitive and specific, and is widely employed to determine if a candidate or marketed drug or pharmaceutical blocks a particular ion channel. Researchers (and industrial laboratories that employ them) employ this approach to examine whether compounds block HERG. Pharmaceutical companies can license mammalian cell lines stably expressing HERG channels for this purpose. The present invention improves on the approaches currently performed in the art by also providing for the expression of KCR1 in the system.

A researcher interested in whether a compound blocks cardiac I_{Kr} can measure HERG current in cells either stably or transiently expressing a HERG channel polypeptide and optionally a hKCR1 as well (and any other proteins suspected of modulating HERG block). The researcher can then assess the IC₅₀ of block by the compound. The researcher can optionally measure the potency of block in cells that only express HERG (and no KCR1) as well as in cells that coexpress HERG and KCR1, to determine if KCR1 expression specifically modulates the drug block.

A company or researcher can decide to pursue (or not pursue) development of a compound based on the findings from these experiments. For example, a compound that exhibited potent HERG block can be excluded from future development regardless of the modulatory effect of KCR1. However, if a compound exhibited relatively mild HERG block (as many do), and KCR1 coexpression further limited that block, the company or researcher could choose to pursue future development on this basis.

III. Molecular Elements of the Present Invention

The following sections disclose some molecular elements of the present invention. The following sections are not meant to be a cumulative list of the elements of the present invention. Some of the protein elements of the present invention are discussed, as well as some HERG channel inhibitors. Details of long QT syndrome, and a discussion of how some of these molecular elements are related to LQT, are presented in section V.

III.A. HERG Channel Polypeptide

The human form of the erg gene, the HERG gene (Genbank Accession Number U04270), which encodes the HERG potassium ion channel subunits was first described by Warmke & Ganetzky, (Warmke & Ganetzky, (1994) Proc. Natl. Acad. Sci. U.S.A. 91: 3438-3442), incorporated herein by reference. A Drosophila erg gene was described by Titus et al. (Titus et al., (1997) J. Neurosci. 17: 875-881; Genbank Accession Number U42204). A C. elegans erg gene (Genbank Accession Number AF257518) has also been identified. A HERG polypeptide sequence is also set forth in GenBank Accession Number BAA37096, and a HERG nucleotide sequence is also set forth in GenBank Accession Number SEG_AB00905S.

In 1994, Warmke and Ganetzky identified a novel human cDNA, human ether a-go-go related gene (HERG) (Warmke & Ganetzky, (1994) Proc Nat'l Acad Sci USA 91: 3438-3442). HERG was localized to human chromosome 7 by PCR analysis of a somatic cell hybrid panel (Warmke & Ganetzky, (1994) Proc Nat'l Acad Sci USA 91: 3438-3442). The function of the protein encoded by HERG was not known, but it has predicted amino acid sequence homology to potassium channels. HERG was isolated from a hippocampal cDNA library by homology to the Drosophila ether a-go-go gene (eag), which encodes a calcium-modulated potassium channel (Bruggemann et al., (1993). Nature 365: 445-448.). HERG is not the human homolog of eag, however, sharing only about 50% amino acid sequence homology. The function of HERG was unknown, but it was strongly expressed in the heart and was hypothesized to play an important role in repolarization of cardiac action potentials and was linked to LQT (Curran et al., (1995) Cell. 81: 299-307).

To define the physiologic role of the polypeptide encoded by HERG, the full-length cDNA was cloned and the channel was expressed in Xenopus oocytes. Voltage-clamp analyses of the resulting currents revealed that HERG encodes a K⁺ channel with biophysical characteristics nearly identical to I_{Kr}. These data suggested that HERG encodes the major subunit for the I_{Kr} channel, and provide a mechanistic link between some forms of inherited and drug-induced LQT.

III.B. KCR1

KCR1, a novel protein recently cloned from a rat cerebellar cDNA library, is widely expressed and modulates the function of ether-à-go-go (EAG) K⁺ channels in the rat cerebellum (Hoshi et al., (1998) J Biol Chem 273: 23080-23085). Although related to HERG (49% amino acid identity), (Warmke & Ganetzky, (1994) Proc Nat'l Acad Sci USA 91: 3438-3442) the gating behavior of EAG channels differs substantially; EAG channels are noninactivating, while HERG channels exhibit striking inward rectification that severely limits the outward current passed by the channel at depolarized membrane potentials (Trudeau et al., (1995) Science 269: 92-95; Smith et al., (1996) Nature 379: 833-836; Spector et al., (1996) J Gen Physiol 107: 611-619). However, both channels possess C-terminal cyclic nucleotide binding domains (cNBD), and have overlapping pharmacologic sensitivities (Weinshenker et al., (1999) J Neurosci 19: 9831-9840; Teschemacher et al., (1999) Br J Pharmacol 128: 479-485).

III.C. MiRP1

MiRP1 is a single transmembrane protein that has been shown to interact with HERG in coimmunoprecipitation assays, and to modulate the functional behavior of HERG, including its conductance and gating properties (Abbott et al., (1999) Cell 97:175-187). In addition, mutations and polymorphisms in the MiRP1 sequence have been shown to enhance the sensitivity of HERG to drug blockade (Sesti, F., et al., Proc Natl Acad Sci USA. 97:10613-8 (2000)). The biophysical mechanism whereby MiRP1 modulates HERG function and pharmacology is uncertain, and thus, the observations of the present invention add significantly to the art.

A MiRP1 polypeptide sequence is set forth in GenBank Accession Number Q9Y6J6, and a MiRP1 nucleotide sequence is set forth in GenBank Accession Number XM_{—}048634.

III.D. HERG Channel Inhibitors

Cardiac potassium channels are blocked by a diverse array of common therapeutic compounds (antihistamines, antidepressants, antibiotics, antiarrhythmics), and exposure to these agents provokes life-threatening cardiac arrhythmias in some, but not all, individuals (Ackerman & Clapham, (1997) N. Engl. J. Med. 336: 11575-1586). However, the molecular factors predicting such wide variability in drug response are not defined (Roden, (1998) Pacing Clin. Electrophysiol. 21: 1029-1034). While these compounds target the principal pore-forming subunits of K⁺ channels, other proteins that associate with K⁺ channels could alter their function (Rettig et al., (1994) Nature 369: 289-294), and could therefore affect drug-channel interactions. Blocking of cardiac channels can lead to cardiac arrhythmia, heart damage and even death.

The list of drugs that block HERG is extremely long, and more are identified almost daily. A web site that contains a partial list is: http://www.ihc.com/research/longgt.html. Representative drugs include but are not limited to: anesthetics/asthma medications (e.g. epinephrine), antihistamines (e.g. terfenadine, astemizole, and diphenhydramine), antibiotics (e.g. erythromycin, trimethoprim, and sulfamethoxazole pentamidine), heart medications (e.g. quinidine, procainamide, disopyramide, sotalol, probucol, bepridil), gastrointestinal medications (e.g. cisapride), antifungal drugs (e.g. ketoconazole, fluconazole, and itraconazole), psychotropic drugs (e.g. amitriptyline (tricyclics), phenothiazine derivatives, haloperidol, risperidone, and pimozide), and diuretics (e.g. indapamide). These representative drugs, as well as others, are implicate in acquired Long QT Syndrome.

IV. Long QT Syndrome

Although sudden death from cardiac arrhythmias is thought to account for 11% of all natural deaths, the mechanisms underlying arrhythmias are poorly understood (Kannel et al., (1987). Am. Heart J. 113: 799-804; Willich et al., (1987). Am. J. Cardiol. 60: 801-806.). One form of long QT syndrome (LQT) is an inherited cardiac arrhythmia that causes abrupt loss of consciousness, syncope, seizures and sudden death from ventricular tachyarrhythmias, specifically torsade de pointes and ventricular fibrillation (Ward, (1964). J. Ir. Med. Assoc. 54, 103-106; Romano, (1965) Lancet 1658-659; Schwartz et al., (1975) Am. Heart J. 109: 378-390; Moss et al., (1991) Circulation 84: 1136-1144.). This disorder usually occurs in young, otherwise healthy individuals (Ward, (1964) J. Ir. Med. Assoc. 54: 103-106; Romano, (1965) Lancet 1658-659; Schwartz et al., (1975) Am. Heart J. 109: 378-390). Most LQT gene carriers manifest prolongation of the QT interval on electrocardiograms, a sign of abnormal cardiac repolarization (Vincent et al., (1992) N. Engl. J. Med. 327: 846-852).

Autosomal dominant and autosomal recessive forms of the hereditary form of this disorder have been reported. Autosomal recessive LQT (also known as Jervell-Lange-Nielsen syndrome) has been associated with congenital neural deafness; this form of LQT is rare (Jervell & Lange-Nielsen, (1957). Am. Heart J. 54: 59-78). Autosomal dominant LQT (Romano-Ward syndrome) is more common, and is not associated with other phenotypic abnormalities.

A more common form of this disorder is called “acquired LQT” and it can be induced by many different factors, particularly treatment with certain medications and reduced serum K⁺ levels (hypokalemia). Thus, acquired LQT is usually a result of pharmacologic therapy (Schwartz et al., (1975). Am. Heart J. 109, 378-390; Zipes, (1987). Am. J. Cardiol. 59: 26E-31E).

While it is not applicants' intention to be bound by any theory of operation, two hypotheses for LQT have previously been proposed (Schwartz et al., (1994). The long QT Syndrome. in Cardiac Electrophysiology: From Cell to Bedside, (Zipes & Jalife, eds.), W. B. Sanders Company, pp.788-811). One suggests that a predominance of left autonomic innervation causes abnormal cardiac repolarization and arrhythmias. This hypothesis is supported by the finding that arrhythmias can be induced in dogs by removal of the right stellate ganglion. In addition, anecdotal evidence suggests that some LQT patients are effectively treated by β-adrenergic blocking agents and by left stellate ganglionectomy (Schwartz et al., (1994). The long QT Syndrome. in Cardiac Electrophysiology: From Cell to Bedside, (Zipes & Jalife, eds.), W. B. Sanders Company, pp.788-811).

The second hypothesis for LQT-related arrhythmias suggests that mutations in cardiac-specific ion channel genes, or genes that modulate cardiac ion channels, cause delayed myocellular repolarization. Delayed myocellular repolarization could promote reactivation of L-type calcium channels, resulting in secondary depolarizations (January & Riddle, (1989). Circ. Res. 64: 977-990). These secondary depolarizations are the likely cellular mechanism of torsade de pointes arrhythmias (Surawicz, (1989). J. Am. Coll. Cardiol. 14: 172-184). This hypothesis is supported by the observation that pharmacologic block of potassium channels can induce QT prolongation and repolarization-related arrhythmias in humans and animal models (Antzelevitch & Sicouri, (1994). J. Am. Col. Card. 23: 259-277). The discovery that one form of LQT results from mutations in a cardiac potassium channel gene supports the myocellular hypothesis.

The clinical features of LQT result from episodic cardiac arrhythmias, specifically torsade de pointes, named for the characteristic undulating nature of the electrocardiogram in this arrhythmia. Torsade de pointes can degenerate into ventricular fibrillation, a particularly lethal arrhythmia. Although LQT is not a common diagnosis, ventricular arrhythmias are very common; more than 300,000 United States citizens die suddenly every year (Kannel et al., (1987). Am. Heart J. 113: 799-804; Willich et al., (1987). Am. J. Cardiol. 60: 801-806) and, in many cases, the underlying mechanism can be aberrant cardiac repolarization. LQT, therefore, provides a unique opportunity to study life-threatening cardiac arrhythmias at the molecular level.

V. Drug Screening Methods

The present invention can be applied in a range of applications. Of particular value to researchers and drug developers are methods by which a candidate pharmaceutical can be tested for its effect on HERG channel activity. Since HERG channel activity is related to long QT syndrome, the methods can assist in the identification of compounds that are likely to give rise to a LQT condition. This ability can minimize the risk to a patient that the patient will suffer LQT-related injury. The methods of the present invention can, therefore, be employed in drug design.

The methods of the present invention can be employed before a drug reaches the marketplace. Alternatively, the methods of the present invention can be employed to identify the propensity of these drugs to give rise to a LQT condition. When the methods of the present invention are applied to a candidate pharmaceutical that is in development, a drug designer or researcher can identify a candidate pharmaceutical that is likely to give rise to a LQT condition and, if desired, remove the candidate from the research program. This can save a drug developer time and money by identifying those candidate compounds that are not worthy of pursuing in clinical trials. Alternatively, if development is pursued, suitable warning to medical practitioners and patients can be provided, based on data derived from the methods of the present invention. Additionally, since the data derived from the methods of the present invention can be quantitative, the methods offer the ability to gauge the relative LQT effect a given candidate might exhibit.

The methods of the present invention can also be applicable to drugs already in the marketplace. In this context, the methods can be employed to identify drugs that can pose a risk of LQT and can be marked as such. Cumulatively, the methods of the present invention offer benefit not only to those developing drugs, but those to whom these and other drugs are administered. Ultimately, the methods of the present invention offer the ability to prevent the injury or even death of a patient.

The following discussion is not meant to be an all-encompassing description of the methods of the present invention. Additionally, although the steps of the various methods are disclosed in the context of one single method, it is understood that the general discussion accompanying the methods is intended to apply to all of the claimed methods. Variations on the disclosed methods can be made fall within the claims and spirit of the present invention. Such variations on the disclosed methods will be apparent to those of skill in the art upon contemplation of the present disclosure.

V.A. Method of Identifying a Compound That Modulates a Biological Activity of a Potassium Channel

Ion channel blockade is often determined by the voltage-gated conformational state of the channel, so that high-throughput screening of compounds for such activity using simple radioligand binding methods is often infeasible and insensitive. The present invention can offer an alternative to these infeasible and insensitive methods.

In one embodiment, an extracellular ion concentration or another intervention (such as an applied electric field, or a compound that alters the membrane potential) can be manipulated to set a membrane potential at a level that will likely change when a test compound binds to a target channel (e.g., a HERG channel). More specifically, stably-transfected reporter cells can be grown in 96-well culture plates and then loaded with a voltage-sensitive dye (e.g., carbocyanides, DiANEPP, diBAC, etc.) with a dynamic range and response time that allows detection of transmembrane voltage. A compound of interest can then be applied to each well of the dish, with the appropriate control also being applied. Transmembrane potential can then be recorded using any of a variety of detection methods, however automated fluorescence detection for multiple samples (e.g., FLIPR technology) is preferred. By assessing the effects of varying concentrations of test compounds in cells that express either HERG alone, HERG and KCR1, or HERG in combination with other proteins, the effect of the compound on HERG biological activity can be assessed. This information on KCR1-modulated HERG biological activity can be employed to determine whether future drug development efforts should be pursued.

In another embodiment, a structure comprising a potassium channel polypeptide and a KCR1 polypeptide is provided. The structure can comprise, for example, a cell expressing both a potassium channel polypeptide and a KCR1 polypeptide. If the structure is a cell, it is preferable that the cell is isolated from a subject. A cell can be acquired from a subject either directly, by removing them from the subject or alternatively, a viable cell line can be employed as a source of cells. It is not necessary that the subject is a human. A subject, and therefore, a cell derived therefrom, can be any living organism. For example, as disclosed in the Laboratory Examples hereinbelow, a Chinese hamster can serve as a subject and thus a source of cells.

Preferably, the KCR1 polypeptide is encoded by a human KCR1 nucleic acid sequence, and more preferably by a nucleic acid sequence comprising SEQ ID NO: 1. It is also preferable, but not required, that the potassium channel polypeptide comprise a HERG channel comprising the polypeptide sequence of SEQ ID NO: 3. It is also preferable that the potassium channel polypeptide and the KCR1 polypeptide form components of the structure. That is, it is preferable that the proteins are embedded in the structure and, if appropriate, span the membrane. It is also particularly preferable, but not required, that the proteins exist in a functional state in the structure. To clarify, it is preferable that the proteins assume conformations and orientations in the structure similar to those conformations and orientations the proteins adopt in vivo.

Alternatively, the structure can comprise a constructed lipid bilayer, which can be a liposome or a planar bilayer. A constructed bilayer can be made by employing standard bilayer preparation methods. When a liposome is selected as a structure, a number of methods are available in the art for preparing liposomes and can be employed (see, e.g., Liposometechnology 2nd ed. Vol. I Liposome preparation and related techniques, (Gregoriadis, ed.) CRC Press, Boca Raton, Fla., 1993; Watwe et al., (1995) Curr. Sci. 68: 715; Vemuri et al.; (1995) Pharm. Acta Helvetiae 70: 95; and U.S. Pat. Nos. 4,737,323; 5,008,050; and 5,252,348). Frequently employed techniques for lipid bilayer construction include, but are not limited to, hydration of a lipid film, injection, sonication and detergent dialysis.

A preferred method of construction comprises sonication (see, e.g., Hub et al., (1980) Angew. Chem. Int. Ed. Engl. 19: 938). This method is easy to use and produces unilamellar spherical vesicles of small and uniform size. Briefly, a thin film of lipid is heated with water above 90° C., and then cooled to about 4° C., which is below the T_{c} (Lopez et al., (1982) Biochim. Biophys. Acta 693: 437) to permit the lipids to form a “solid analogous” state. The mixture is then sonicated for several minutes, with longer times typically producing more uniform vesicles. After formation, the vesicles can be reduced in size, if desired, by freeze-thaw cycles or extruding through filters of progressively smaller pore size.

Next, a test compound can be contacting with the structure. The contacting can be performed under virtually any conditions. It is preferable, however, that the contacting be done under sterile, controlled conditions in order to minimize the likelihood of contamination. The exact mechanism of the contacting is also variable and can rely, at least in part, on the properties of the compound. For example, if the compound is suspended in a liquid, the liquid itself can be contacted with the structure.

A biological activity of the potassium channel polypeptide in the presence of the test compound can then be determined. The biological activity can comprise any biological activity associated with the potassium channel (e.g., association with a secondary component, inhibitor or activator binding, etc.), however a preferred biological activity comprises transport of potassium ions.

When a biological activity is potassium ion transport, the determining can be performed by measuring a voltage or current across the structure. Typically, such measurements are performed by employing patch clamp technology, which is also described elsewhere herein.

In the context of the present invention, patch clamp experiments can be performed by employing an Axopatch 200B amplifier (Axon Instrrnents, Burlingame, Calif.) linked to an IBM compatible personal computer equipped with pCLAMP software. Patch-clamp experiments can be performed at room temperature (21-23° C.), following standard procedures, such as those set forth in Sakmann & Neher, (1983) Single Channels Recordings, Plenum Press, New York, N.Y. and in Kukuljan et al., (1991) J. Membrane Biol. 119: 187. The general protocol for employing the amplifier can be based on the aforementioned references, as well as guidelines supplied by the manufacturer; precise details of a suitable procedure will be apparent to those of skill in the art upon contemplation of the present disclosure.

The biological activity of the potassium channel polypeptide in the presence of the test compound can then be compared to the biological activity of the potassium channel polypeptide in an absence of the test compound. The comparison can comprise a statistical comparison or it can comprise a simple numerical comparison of determined activity values.

The comparison of the activity values can provide an assessment of a degree of biological activity modulation imparted by the test compound. For example, a difference between the biological activity of the potassium channel in the absence of the test compound and the biological activity of the potassium channel polypeptide in the presence of test compound indicates modulation of a biological activity of the potassium channel. Additionally, the comparison can yield a quantitative difference in biological activity that is affected by the test compound.

V.B. Method of Identifying a Candidate Compound as a HERG Channel Inhibitor

In another aspect, the present invention discloses a method of identifying a candidate compound as a HERG channel inhibitor. As disclosed herein, there is a need in the pharmaceutical and other industries to be able to identify a candidate compound as a HERG channel inhibitor. This ability can be employed at the early stages of pharmaceutical development and can allow a researcher to identify risks associated with a candidate pharmaceutical at an early stage of development and well before costly clinical trials.

As noted, many common therapeutics are HERG channel inhibitors. Some of these therapeutics were designed as HERG channel inhibitors, while others exhibit HERG channel inhibition as an undesired side effect. In many cases, this undesired side effect does not become known until clinical trials are underway and sometimes not even until severe harm or death befalls a member of the general public.

The present method offers an alternative to researchers and those engaged in pharmaceutical research and development. By employing the present method, a candidate therapeutic can be identified as a HERG channel inhibitor before it reaches the stage where it is administered to a subject. Thus, the method can fill a vital role in a research program, particularly if a goal of the research program is to provide a pharmaceutical that does not block HERG channels. Alternatively, if the pharmaceutical is identified as a HERG channel inhibitor, the pharmaceutical can be contraindicated for those who are afflicted with inherited long QT syndrome, in which subjects the pharmaceutical might impart an unacceptable risk factor.

In a preferred embodiment, the first step of the method comprises providing a structure comprising a HERG potassium channel and a KCR1 polypeptide. As disclosed above, it is preferable that the structure comprises a cell or a lipid bilayer. Both can be prepared as disclosed elsewhere herein.

Again, it is preferable that the KCR1 polypeptide is encoded by a human KCR1 nucleic acid sequence, and more preferably by a nucleic acid sequence comprising SEQ ID NO: 1. It is also preferable, but not required, that the potassium channel polypeptide comprise a HERG channel comprising the polypeptide sequence of SEQ ID NO: 3. It is also preferable that the potassium channel polypeptide and the KCR1 polypeptide form components of the structure. That is, it is preferable that the proteins are embedded in the structure and, if appropriate, span the membrane. It is also particularly preferable, but not required, that the proteins exist in a functional state in the structure. To clarify, it is preferable that the proteins assume conformations and orientations in the structure similar to those conformations and orientations the proteins adopt in vivo.

A candidate compound can then be contacted with the structure. In a preferred embodiment, the contacting can be performed by dripping a solution comprising the candidate compound over the structure. The contacting can be performed in a sterile environment and/or an environment in which conditions are controlled and maintained at levels which preserve the integrity of the structure. Various methods of contacting can be employed in the present invention and will be apparent to those of skill in the art upon consideration of the present invention.

A biological activity of the HERG potassium channel is then determined in the presence of the candidate compound. The method of the determination can be dictated, in part, by the nature of the biological activity. Preferably, but not necessarily, a biological activity is transport of potassium ions. When transport of potassium ions is a biological activity, the biological activity can be detected via detection of a voltage or current, which can accompany transport of potassium ions. Such a current can be detected, and this biological activity determined by employing a patch clamp apparatus, such as the patch clamp apparatus disclosed above.

Once a biological activity of the HERG potassium channel is determined in the presence of the candidate compound, that activity can be compare with HERG potassium channel activity in an absence of the candidate compound. The comparison is preferably a quantitative comparison, and can optionally involve a statistical analysis. When practicing the present method, or any of the methods of the present invention comprising a comparison between two or more values, a statistical analysis can be performed. Additionally, a statistical analysis can comprise a plurality of activity determinations. In fact, it is preferable, but not necessary, that a plurality of determinations be made. By acquiring a plurality of determinations, a more complete assessment of a biological activity can be performed.

An analysis of acquired data can then be performed. In this method the candidate compound can be identified as a HERG potassium channel inhibitor if the biological activity of the HERG potassium channel in the presence of the candidate compound is lower than the biological activity of the HERG potassium channel in the absence of the candidate compound.

V.C. Method of Predicting a Propensity of a Candidate Drug to Induce Cardiac Arrhythmia

The following method offers the ability to predict the propensity of a candidate drug to induce cardiac arrhythmia. This ability can be of immense value to drug designers, who are continuously assessing the safety of the drugs they develop. A drug designer can employ the present method to identify a candidate drug that poses a risk to a patient of cardiac arryhmia, which can lead to injury or death. Additionally, the methods permits drug developers to remove unacceptably dangerous drugs from development and can save time and money by identifying a compound that is unsuitable for clinical trials.

In a preferred embodiment of the method, a structure comprising a potassium channel and a KCR1 polypeptide is provided. As noted herein, the structure can comprise a cell or a lipid bilayer. Both structures offer advantages and the selection of one over another can be dependent, in part, on the nature of the determination to be performed. For example, a structure comprising a lipid bilayer offers the advantages that it can be conveniently prepared in a laboratory and does not require isolation of a cell from a subject. In practice, a lipid bilayer can be prepared de novo or can even be isolated from another organism, such as a prokaryote. Preferably, the potassium channel polypeptide comprises a HERG channel. Alternatively, another potassium channel, e.g. a potassium channel derived from an organism other than a human, can be employed.

Again, it is preferable that the KCR1 polypeptide is encoded by a human KCR1 nucleic acid sequence, and more preferably by a nucleic acid sequence comprising SEQ ID NO: 1. It is also preferable, but not required, that the potassium channel polypeptide comprise a HERG channel comprising the polypeptide sequence of SEQ ID NO: 3. It is also preferable that the potassium channel polypeptide and the KCR1 polypeptide form components of the structure. That is, it is preferable that the proteins are embedded in the structure and, if appropriate, span the membrane. It is also particularly preferable, but not required, that the proteins exist in a functional state in the structure. To clarify, it is preferable that the proteins assume conformations and orientations in the structure similar to those conformations and orientations the proteins adopt in vivo.

Next, a candidate drug is contacted with the structure. The contacting can be achieved in any convenient and feasible way. For example, a candidate drug can be suspended in a solution and the solution can be dripped onto the structure. Alternatively, the structure can be placed in a bathing solution and the candidate drug can be added to the bathing solution.

Subsequently, a biological activity of the potassium channel in the presence of the candidate drug is determined. This determination can be made by employing the techniques disclosed herein. For example, a preferred biological activity comprises potassium ion transport. For this biological activity, patch clamp or ion flux comprise preferred assays can be employed to determine a biological activity.

Finally, the biological activity of the potassium channel in the presence of a KCR1 polypeptide and in an absence of a candidate drug is compared to a biological activity of the potassium channel in the presence of the candidate drug. The biological activity of the potassium channel in the absence of the candidate drug is preferably determined by employing the same techniques that were employed to determine the biological activity in the presence of the candidate drug (e.g., patch clamp or ion flux techniques). Preferably, this determination can be made just prior to the determination of activity in the presence of the candidate drug. However, the activity of the channel in the absence of the candidate drug can also be determined well ahead of time or can comprise a standard reference activity, eliminating the need for a researcher to perform the assay.

The analysis of the comparison can provide data on the propensity of a candidate drug to induce cardiac arrhythmia. Specifically, if a biological activity of the potassium channel in the presence of a candidate drug is less than a biological activity of the potassium channel in an absence of the candidate drug, this observation is indicative of a propensity of the drug to induce cardiac arrhythmia in a subject.

When a candidate drug is found to have a propensity to induce cardiac arrhythmia in a subject, this information can play a role in a decision regarding whether to pursue research on the candidate. If a candidate drug is found to not exhibit a propensity to induce cardiac arrhythmia in a subject, the drug can be pursued in development and clinical trial with the confidence that it does not pose an acquired LQT risk to patients. Conversely, if a candidate drug is found to have a propensity to induce cardiac arrhythmia, it can be removed from further development protocols.

VI. Techniques and Reagents Useful for Practicing the Methods of the Present Invention

The following section discloses several assays and techniques that are useful from practicing the methods of the present invention. This discussion is meant to be representative; and, those of skill in the art, upon consideration of the present disclosure, will recognize additional assays and techniques that will be useful.

A common technique for monitoring ion flow through a pore comprises patch clamp, or voltage clamp, methods. These methods are described hereinbelow. Additionally, methods of preparing cells and lipid bilayers, both of which can be employed in the present invention, are also disclosed. An ion flux assay, which can be employed exclusive of, or in conjunction with, a patch clamp-based study is further disclosed. Moreover, a system for heterologous expression of a HERG channel polypeptide and/or a KCR1 polypeptide, an aspect of the present invention, is disclosed. The following sections further disclose sequences substantially similar to those of SEQ ID NOs: 1 to 5.

VI.A. Patch Clamp Techniques

The clamp technique and improvements thereof, have been developed to study electrical currents in cells. The technique is commonly employed to study ion transfer through channels. To measure these currents, the membrane of the cell is closely attached to the opening of the patch micropipette so that a very tight seal is achieved. This seal prevents current from leaking outside of the patch micropipette. The resulting high electrical resistance across the seal can be exploited to perform high resolution current measurements and apply voltages across the membrane. Different configurations of the patch clamp technique can be employed. (Sakmann & Neker, (1984) Ann. Rev. Physiol. 46: 455).

Any host cell (including heterologous cells) can be used for patch clamp analysis, including but not limited to PC12 cells (D'Arcangelo et al., (1993) J. Cell Biol. 122(4): 915-921.), Xenopus oocytes (Stuhmer et al., (1989) EMBO J. 8(11): 3235-3244 (1989).; Taglialatela et al., (1992) Biophys J 61: 78-82; Ji et al., (1999) J Biol Chem 274: 37693-37704), Chinese hamster ovary (CHO) cells (Dupere et al., (1999) Br J Pharmacol 128: 1011-1020), HEK-293 human kidney cell (Sabirov et al., (1999) J Membr Biol 172: 67-76), and Sf9 insect cells Yamashita et al., (1999) Eur J Pharmacol 378: 223-231). For selective study of K⁺ currents mediated by recombinant expression of a HERG potassium channel polypeptide and/or a KCR1 polypeptide, a host cell is preferably free of endogenous potassium channels.

Optionally, whole-cell patch clamp technique can be combined with single cell RT-PCR to confirm the causal relationship between recombinant HERG potassium channel and/or a KCR1 expression and K⁺ conductance. See Chiang, (1998) J Chromatogr A 806: 209-218, and references cited therein.

VI.B. Ion Flux Assay

A candidate substance can be tested for its ability to modulate a potassium channel by determining the influx of ion tracers through the channel. Representative labeled potassium ions that can be employed to assay channel conductance include but are not limited to ⁴¹K. Briefly, aliquots of a cell suspension comprising heterologous cells expressing a potassium channel are incubated for 10 minutes at 37° C. in the presence of channel openers and test substances in a total volume of 100 μM (0.20-0.25 mg protein). Ion flux is initiated by the addition of HEPES/TRIS solution also containing 4 mM guanidine HCl (final) and 1000 dpm/nmol ¹⁴C guanidine. The reaction is continued for 30 seconds and is stopped by the addition of ice-cold incubation buffer, followed by rapid filtration under vacuum over a glass microfiber filter (grade GF/C, 1.2 μm available from Whatman, Inc. of Clifton, N.J.). The filters are washed rapidly with ice-cold incubation buffer and radioactivity is determined by scintillation counting. Nonspecific uptake can be determined in parallel reactions.

As described herein, an ion flux assay can further comprise contacting a cell expressing a HERG channel polypeptide and/or a KCR1 polypeptide with a test substance and a known HERG channel modulator. For example, substantial ion flux is observed in the presence of a persistent potassium channel activator, and a reduction of flux following subsequent application of a test substance indicates an antagonist activity of the test substance. Similarly, observation of enhanced ion flux of an already-activated HERG channel following application of a test substance indicates an agonist activity of the test substance.

VI.C. System for HERG Channel Polypeptide Expression and/or HERG/KCR1 Polypeptide Coexpression

The present invention further provides a system for heterologous expression of a functional human HERG channel polypeptide and/or coexpression of a functional HERG channel polypeptide and a functional KCR1 polypeptide. Preferably, the recombinantly expressed human HERG channel polypeptide comprises a functional potassium channel. Thus, a recombinantly expressed HERG channel polypeptide preferably displays voltage-gated ion conductance across a lipid bilayer or membrane. Also preferably, a recombinant HERG channel polypeptide shows activation and inactivation kinetics similar to a native HERG potassium channel polypeptide and/or a KCR1 polypeptide.

In one embodiment of the invention, a system for heterologous expression of a functional human HERG channel polypeptide and/or coexpression of a functional HERG channel polypeptide and a functional KCR1 polypeptide can comprise: (a) a recombinantly expressed HERG channel polypeptide and/or a KCR1 polypeptide; and (b) a host cell comprising the recombinantly expressed HERG channel polypeptide and/or a KCR1 polypeptide.

In another embodiment of the invention, a system for heterologous expression of a functional human HERG channel polypeptide and/or coexpression of a functional HERG channel polypeptide and a functional KCR1 polypeptide comprises: (a) a vector comprising a nucleic acid molecule encoding a human HERG channel polypeptide operatively linked to a heterologous promoter; (b) a vector comprising a nucleic acid molecule encoding a human KCR1 polypeptide operatively linked to a heterologous promoter; and (c) a host cell comprising the vector of (a), and/or the vector of (b) wherein the host cell expresses a human HERG channel and a KCR1 polypeptide. One vector can comprise both a nucleic acid molecule encoding a human HERG channel polypeptide operatively linked to a heterologous promoter and a nucleic acid molecule encoding a human KCR1 polypeptide operatively linked to a heterologous promoter.

A construct for coexpression of a HERG channel polypeptide and/or a KCR1 polypeptide includes one or more vectors and one or more nucleotide sequences encoding a HERG channel polypeptide and/or a KCR1 polypeptide, wherein the nucleotide sequence(s) is operatively linked to a promoter sequence. Recombinant production of a HERG channel polypeptide and/or a KCR1 polypeptide can be directed using a constitutive promoter or an inducible promoter. Exemplary promoters include Simian virus 40 (SV40) early promoter, a long terminal repeat promoter from retrovirus, an actin promoter, a heat shock promoter, and a metallothien protein. Suitable vectors that can be used to express a HERG channel polypeptide and/or a KCR1 polypeptide include, but are not limited to, viruses such as vaccinia virus or adenovirus, baculovirus vectors, yeast vectors, bacteriophage vectors (e.g., lambda phage), plasmid and cosmid DNA vectors, transposon-mediated transformation vectors, and derivatives thereof. A construct for recombinant expression can also comprise transcription termination signals and sequences required for proper translation of the nucleotide sequence. Addition of such sequences will be known to those of skill in the art, upon contemplation of the present disclosure.

In a preferred embodiment of the invention, a construct for recombinant expression of a HERG channel polypeptide and/or a KCR1 polypeptide comprises a plasmid vector and one or more nucleic acid sequences encoding a HERG channel polypeptide and/or a KCR1 polypeptide, wherein the nucleic acid(s) is operatively linked to a CMV promoter. Preferably, a nucleic acid encoding a HERG potassium channel polypeptide comprises: (a) one or more nucleotide sequences encoding the polypeptide sequence of SEQ ID NO: 3, or (b) one or more nucleotide sequences substantially identical thereto. Preferably, a nucleic acid encoding a KCR1 polypeptide comprises: (a) one or more nucleotide sequences comprising the nucleotide sequences of SEQ ID NO: 1, or (b) one or more nucleotide sequences substantially identical to SEQ ID NO: 1.

Constructs are transfected into a host cell using a method compatible with the vector employed. Standard transfection methods include electroporation, DEAE-Dextran transfection, calcium phosphate precipitation, liposome-mediated transfection, transposon-mediated transformation, infection using a retrovirus, particle-mediated gene transfer, hyper-velocity gene transfer, and combinations thereof.

A host cell strain can be chosen which modulates the expression of the recombinant sequence, or modifies and processes the gene product in the specific fashion desired. For example, different host cells have characteristic and specific mechanisms for the translational and post-transactional processing and modification (e.g., glycosylation, phosphorylation of proteins, etc.). Appropriate cell lines or host systems can be chosen to ensure the desired modification and processing of the foreign protein expressed. For example, expression in a bacterial system can be used to produce a non-glycosylated core protein product, and expression in yeast will produce a glycosylated product.

In a preferred embodiment of the invention, a HERG potassium channel polypeptide and/or a KCR1 polypeptide is expressed following transient transfection of CHO cells as described in the Laboratory Examples.

The present invention further encompasses recombinant expression of a HERG potassium channel polypeptide and a KCR1 polypeptide in a stable cell line. Methods for generating a stable cell line are described in the Laboratory Examples. Thus, transformed cells, tissues, or non-human organisms are understood to encompass not only the end product of a transformation process, but also transgenic progeny or propagated forms thereof.

In one embodiment of the invention, a system for heterologous expression of a HERG potassium channel polypeptide and/or a KCR1 polypeptide comprises a host cell expressing a native potassium channel or subunit thereof. In another embodiment of the invention, a system for heterologous expression of a HERG potassium channel polypeptide and/or a KCR1 polypeptide comprises a host cell co-transfected with a construct whereby a HERG potassium channel polypeptide and/or a KCR1 polypeptide is recombinantly expressed.

The present invention further encompasses cryopreservation of cells expression a recombinant HERG potassium channel polypeptide and/or a KCR1 polypeptide as disclosed herein. Thus, transiently transfected cells and cells of a stable cell line expressing a HERG potassium channel polypeptide and a KCR1 polypeptide can be frozen and stored for later use.

Cryopreservation media generally consists of a base medium, cryopreservative, and a protein source. The cryopreservative and protein protect the cells from the stress of the freeze-thaw process. For serum-containing medium, a typical cryopreservation medium can be prepared as complete medium containing 10% glycerol; complete medium containing 10% DMSO (dimethylsulfoxide), or 50% cell-conditioned medium with 50% fresh medium with 10% glycerol or 10% DMSO. For serum-free medium, typical cryopreservation formulations include 50% cell-conditioned serum free medium with 50% fresh serum-free medium containing 7.5% DMSO; or fresh serum-free medium containing 7.5% DMSO and 10% cell culture grade DMSO. Preferably, a cell suspension comprising about 10⁶ to about 10⁷ cells per ml is mixed with cryopreservation medium.

Cells are combined with cryopreservation medium in a vial or other container suitable for frozen storage, for example NUNC® CRYOTUBES™ (available from Applied Scientific of South San Francisco, Calif.). Cells can also be aliquotted to wells of a multi-well plate, for example a 96-well plate designed for high-throughput assays, and frozen in plated format.

Cells are preferably cooled from room temperature to a storage temperature at a rate of about −1° C. per minute. The cooling rate can be controlled, for example, by placing vials containing cells in an insulated water-filled reservoir having about 1 liter liquid capacity, and placing such cube in a −70° C. mechanical freezer. Alternatively, the rate of cell cooling can be controlled at about −1C per minute by submersing vials in a volume of liquid refrigerant such as an aliphatic alcohol, the volume of liquid refrigerant being more than fifteen times the total volume of cell culture to be frozen, and placing the submersed culture vials in a conventional freezer at a temperature below about −70° C. Commercial devices for freezing cells are also available, for example, the Planer Mini-Freezer R202/200R (Planer Products Ltd. of Great Britain) and the BF-5 Biological Freezer (Union Carbide Corporation of Danbury, Conn.). Preferably, frozen cells are stored at or below about −70° C. to about −80° C., and more preferably at or below about −130° C.

To obtain the best possible survival of the cells, thawing of the cells must be performed as quickly as possible. Once a vial or other reservoir containing frozen cells is removed from storage, it should be placed directly into a 37° C. water bath and gently shaken until it is completely thawed. If cells are particularly sensitive to cryopreservatives, the cells are centrifuged to remove cryopreservative prior to further growth.

Additional methods for preparation and handling of frozen cells can be found in Freshney, (1987) Culture of Animal Cells: A Manual of Basic Technique, 2nd ed. A. R. Liss, New York and in U.S. Pat. Nos. 6,176,089; 6,140,123; 5,629,145; and 4,455,842; among other places.

Isolated polypeptides and recombinantly produced polypeptides can be purified and characterized using a variety of standard techniques that are known to the skilled artisan. See, e.g., Schröder & Lübke, (1965) The Peptides. Academic Press, New York; Schneider & Eberle (1993) Peptides, 1992: Proceedings of the Twenty-Second European Peptide Symposium, Sep. 13-19, 1992, Interlaken, Switzerland. Escom, Leiden; Bodanszky (1993) Principles of Peptide Synthesis, 2^{nd} rev. ed. Springer-Verlag, Berlin; New York; Ausubel (ed.) (1995) Short Protocols in Molecular Biology, 3rd ed. Wiley, New York, N.Y.

VI.D. Sequence Similarity and Identity

As used herein, the term “substantially similar” as applied to a HERG potassium channel and/or a KCR1 polypeptide means that a particular sequence varies from nucleic acid sequence of SEQ ID NO: 1, or the amino acid sequence of SEQ ID NOs: 2 or 3 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

VI.D.1. Sequences That are Substantially Identical to a HERG and/or a KCR1 Polypeptide and/or Polynucleotide Sequence of the Present Invention

Nucleic acids that are substantially identical to a nucleic acid sequence of a HERG potassium channel and/or a KCR1 polypeptide of the present invention, e.g. allelic variants, genetically altered versions of the gene, etc., bind to a HERG potassium channel- and/or a KCR1 polypeptide-encoding sequence under stringent hybridization conditions. By using probes, particularly labeled probes of DNA sequences, one can isolate homologous or related genes. The source of homologous genes can be any species, e.g. primate species; rodents, such as rats and mice, canines, felines, bovines, equines, yeast, nematodes, etc.

Between mammalian species, e.g., human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 18 nt long, more usually at least about 30 nt long, and can extend to the complete sequence that is being compared. Algorithms for sequence analysis are known in the art, such as BLAST, described in Altschul et al., (1990) J. Mol. Biol. 215: 403-10. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix. See Henikoff & Henikoff, (1989) Proc Natl Acad Sci U.S.A. 89: 10915.

In addition to calculating percent sequence identity, the BLAST algorithm also performs a statistical analysis of the similarity between two sequences. See, e.g., Karlin and Altschul, (1993) Proc Natl Acad Sci U.S.A. 90: 5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids) which are similar, divided by the total number of symbols in the shorter of the two sequences. The preferred parameters for the GAP program are the default parameters, which do not impose a penalty for end gaps. See, e.g., Schwartz et al., eds., (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NO: 1 or from a nucleotide sequence encoding SEQ ID NO: 3; or (b) the DNA analog sequence is capable of hybridization with DNA sequences of (a) under stringent conditions and which encode a biologically active HERG potassium channel polypeptide and/or a KCR1 polypeptide; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6× SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, preferred conditions are salt concentration of about 200 mM and temperature of about 45° C. One example of such stringent conditions is hybridization at 4× SSC, at 65° C., followed by a washing in 0.1× SSC at 65° C. for one hour. Another exemplary stringent hybridization scheme uses 50% formamide, 4× SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1× SSC (9 mM NaCl/0.9 mM sodium citrate) and the sequences will remain bound when subjected to washing at 55° C. in 1× SSC.

VI.D.2. Complementarity and Hybridization to a HERG and/or a KCR1 Polypeptide and/or Polynucleotide Sequence

As used herein, the term “complementary sequences” means nucleic acid sequences that are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, N.Y. Other hybridization conditions are disclosed above.

VI.D.3. Functional Equivalents of an HERG and/or a KCR1 Polypeptide and/or Nucleic Acid Sequence

As used herein, the term “functionally equivalent codon” is used to refer to codons that encode the same amino acid, such as the ACG and AGU codons for serine. For example, HERG potassium channel-encoding nucleic acid sequences encoding SEQ ID NO:3 and/or a KCR1-encoding nucleic acid sequences comprising SEQ ID NO: 1 that have functionally equivalent codons are covered by the present invention. Thus, when referring to the sequence example presented in SEQ ID NOs: 1-3, applicants provide substitution of functionally equivalent codons into the sequence example of SEQ ID NOs: 1-3. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

VI.D.4. Biological Equivalents

The present invention envisions and includes biological equivalents of a HERG potassium channel and/or a KCR1 polypeptide and/or a polynucleotide encoding either of the foregoing. The term “biological equivalent” refers to proteins having amino acid sequences which are substantially identical to the amino acid sequence of a HERG potassium channel polypeptide and/or a KCR1 polypeptide of the present invention and which are capable of exerting a biological effect, such as transporting potassium ions, binding small molecules or cross-reacting with anti-HERG potassium channel polypeptide and/or a KCR1 polypeptide antibodies raised against a HERG potassium channel polypeptide and/or a KCR1 polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of a HERG potassium channel polypeptide and/or a KCR1 polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs: 2, 3 and 5, applicants envision substitution of codons that encode biologically equivalent amino acids, as described herein, into the sequence example of SEQ ID NOs: 2, 3 and 5, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of lie for Leu. Changes designed by man can be introduced through the application of site-directed mutagenesis techniques, e.g., to introduce improvements to the antigenicity of the protein or to test an engineered mutant polypeptide of the present invention in order to modulate lipid-binding or other activity, at the molecular level.

Amino acid substitutions, such as those which might be employed in modifying an engineered mutant polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Those of skill in the art will appreciate other biologically functionally equivalent changes. It is implicit in the above discussion, however, that one of skill in the art can appreciate that a radical, rather than a conservative substitution is warranted in a given situation. Non-conservative substitutions in a HERG potassium channel polypeptide and/or a KCR1 polypeptide of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within±2 of the original value is preferred, those which are within±1 of the original value are particularly preferred, and those within±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within±1 of the original value are particularly preferred, and those within±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 1-5. Recombinant vectors and isolated DNA segments can therefore variously include a HERG potassium channel polypeptide- and/or a KCR1 polypeptide-encoding region itself, include coding regions bearing selected alterations or modifications in the basic coding region, or include larger polypeptides which nevertheless comprise a HERG potassium channel polypeptide- and/or a KCR1 polypeptide-encoding regions or can encode biologically functional equivalent proteins or polypeptides which have variant amino acid sequences. Biological activity of a HERG potassium channel polypeptide and/or a KCR1 polypeptide can be determined, for example, by assays disclosed herein.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore provided that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs: 1 and 4, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the present invention encompass biologically functional equivalents of HERG potassium channel and/or KCR1 polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of an engineered mutant of the present invention in order to examine the degree of potassium ion transport activity, or other activity at the molecular level. Various site-directed mutagenesis techniques are known to those of skill in the art and can be employed in the present invention.

The invention further encompasses fusion proteins and peptides wherein a coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a HERG potassium channel and/or a KCR1 gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages are gained by positioning the coding DNA segment under the control of a recombinant, or heterologous, promoter. As used herein, a recombinant or heterologous promoter is a promoter that is not normally associated with a HERG potassium channel and/or a KCR1 gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al., (1989) Molecular Cloning: A Laboratory Manual, Cold Spring Harbor Laboratory, New York, specifically incorporated herein by reference). The promoters employed can be constitutive or inducible and can be used under the appropriate conditions to direct high level expression of the introduced DNA segment, such as is advantageous in the large-scale production of recombinant proteins or peptides. One preferred promoter system provided for use in high-level expression is a T7 promoter-based system.

The above presented discussions of Section VI.D. concerning sequence identity, biological equivalents and the like are equally applicable to the MiRP1 sequences disclosed herein. Representative MiRP1 nucleic acid and polypeptide sequences are set forth herein as SEQ ID NOs: 4 and 5, respectively.

VII. Screening for Modulators of HERG and/or KCR1 Biological Activity

In accordance with the present invention, also provided are methods of screening for modulators of the biological activity of HERG and/or KCR1. As used herein, the term “modulator” means an agent that effects an increase, decrease, or other alteration of any, or all, chemical and biological activities or properties of a HERG polypeptide and/or KCR1 polypeptide, including expression levels. The term “modulation” as used herein refers to both upregulation (i.e., activation or stimulation) and downregulation (i.e. inhibition or suppression) of a response.

VII.A. Method of Identifying a Candidate Compound that Modulates the Biological Activity of a Complex Comprising a HERG Channel Polypeptide and a KCR1 Polypeptide

In another embodiment of the present invention, a candidate compound that modulates the biological activity of a complex comprising a HERG channel polypeptide and a KCR1 polypeptide is identified. This application of the present invention relies, in part, on the observation that a complex comprising a HERG channel polypeptide and a KCR1 polypeptide can modulate HERG channel block imparted by a drug or other moiety. The present disclosure is the first disclosure of this observation, and forms a basis for several of the methods disclosed herein.

In another aspect of the present invention, a method of screening compounds to identify a compound that is useful in treating of preventing long QT syndrome is disclosed. As discussed herein, long QT syndrome can cause injury or death in a patient. It would be of great value to be able to develop a compound capable or treating or preventing long QT syndrome. In acquired LQT, the cause and effect of LQT typically accompanies administration of a medication. This problem, which is associated with many common therapeutics, including antihistamines and antidepressants, can be averted by administering a compound identified by the present method.

The present method can be employed to identify a compound that can be useful to treat or prevent LQT. When treating LQT, such a compound could be administered after symptoms of LQT have appeared. When an identified compound is employed to prevent LQT the compound can be administered prior to administration of a therapeutic known or suspected of contributing to LQT. Alternatively, the compound can be coadministered with a therapeutic known or suspected of contributing to LQT.

In this method, a cell comprising a HERG channel polypeptide and a KCR1 polypeptide is placed into a bathing solution. The cell can be any type of cell that is expressing a HERG channel polypeptide and a KCR1 polypeptide. In a preferred embodiment, the cell is a human cell. In another preferred embodiment, the cell is a Chinese hamster ovary cell. It is also preferable that the cell does not express any endogenous potassium channels, such as HERG homologs or channels with greater than about 45% sequence similarity with a HERG or a human KCR1 polypeptide. Thus, the cell can comprise a heterologous expression system. A preferred bathing solution can comprise 145 mM NaCl, 4 mM KC1, 1.8 mM CaCl₂, 1.0 mM MgCl₂, 10 mM HEPES and 10 mM glucose, pH 7.35.

An induced K⁺ current in the cell can then be determined. As noted above, preferred techniques for measuring a K⁺ current in the cell can comprise an ion flux assay or, more preferably, a patch clamp assay. Both of these assays are described herein and in the Laboratory Examples.

After determining a K⁺ current in the cell (i.e. a current in the absence of a candidate drug), a candidate drug can be added to the bathing solution. The addition can be performed in a variety of ways and can depend, in part, on the form of the drug. For example, a candidate drug can be dissolved in a suitable buffered solution (e.g., a pharmaceutically acceptable diluent) and added to the bathing solution in liquid form. Alternatively, a candidate drug can be added in powdered form and can be dissolved in the bathing solution itself. It is preferable that the candidate drug is added to the bathing solution under sterile and controlled conditions.

After addition of the candidate drug to the bathing solution, an induced K⁺ current in the cell can then be determined (i.e. a current in the presence of a candidate drug). Preferably, the determining of the current is performed by employing the same methodology as was employed to determine the current in the absence of the candidate drug.

After both current measurements are determined (i.e, current in the presence and absence of a candidate drug), a comparison can be made. The comparison can comprise a direct numerical comparison without any treatment of the data or it can comprise a statistical comparison. For such a comparison, or for another comparison, is can be preferable to acquire multiple current measurements and to subsequently perform an averaging or other mathematical operation on the data.

The comparison can reveal an effect of a candidate drug on HERG/KCR1-mediated K⁺ transport. For example, it can be determined that the candidate compound modulates the biological activity of a complex comprising a HERG polypeptide and a KCR1 complex if the current determined in the absence of the candidate drug is different from the current determined in the presence of the candidate drug. Again, this comparison can comprise a statistical analysis to determine, among other properties, the significance of the difference and can assist in interpreting acquired current data.

In a preferred embodiment, a cell is initially transfected with a nucleic acid sequence encoding a HERG channel polypeptide and a nucleic acid sequence encoding a KCR1 polypeptide. Preferably the cell is a human cell, in view of the fact that HERG is derived from humans. In another aspect of the present invention, however, a heterologous expression system is disclosed and thus the cell can be, for example, a Chinese hamster ovary cell. It is preferable that the cell does not express any endogenous potassium channels and that it does not express KCR1 or a homolog (or ortholog) thereof.

It is also preferable that the KCR1 polypeptide is encoded by a human KCR1 nucleic acid sequence, and more preferably by a nucleic acid sequence comprising SEQ ID NO: 1. It is also preferable, but not required, that the potassium channel polypeptide comprise a HERG channel comprising the polypeptide sequence of SEQ ID NO: 3. As discussed elsewhere herein, it will be understood that equivalents of SEQ ID NOs: 1-3 are encompassed by the present invention.

Transfection can be performed by any convenient technique. A variety of transfection techniques are known in the art and can be employed in the present invention. For example, electroporation and calcium phosphate precipitation can be employed. Additional transfection techniques are disclosed herein above and in the Laboratory Examples and can be employed to effect the transfection.

Following transfection the cell is placed into a bathing solution. Representative bathing solutions are disclosed herein and can be employed in the present method. For example, a preferred bathing solution can comprise 145 mM NaCl, 4 mM KC1, 1.8 mM CaCl₂, 1.0 mM MgCl₂, 10 mM HEPES and 10 mM glucose, pH 7.35.

An induced K⁺ current in the cell can then be determined (i.e., induced current in the absence of a candidate drug). Preferably, the determination is performed by employing an ion flux assay or a patch clamp assay as disclosed herein. It is possible, however, to determine an induced K⁺ current by employing any of a range of techniques adapted to generate such measurements.

A candidate drug is then added to the bathing solution. As described in the context of the various methods of the present invention, the drug can be added directly to the bathing solution as a powder or other solid form, or it can be added to the bathing solution in the form of a suspension in a pharmaceutically acceptable liquid.

An induced K⁺ current in the cell is then determined (i.e., induced current in the cell in the presence of the candidate drug). Again, it is preferable that the determination be performed by the same technique as was employed to determine the induced current in the cell in the absence of the candidate drug (e.g., ion flux assay, patch clamp assay, etc.).

After both determinations have been performed, the two values can be compared. The determinations can be interpreted as follows: if the current determined in the presence of the candidate drug is less than the current determined in the absence of the candidate drug, the candidate drug might be useful in treating or preventing long QT syndrome. This conclusion can be drawn based on the fact that LQT is generally attributed to a blocking of HERG channels and thus a greater flux of potassium ions through the channel; a candidate drug that is found to decrease the flux of potassium ions through the channel can thus attenuate LQT. However, if the current through the HERG channel is greater in the presence of the candidate drug than in the absence of the candidate drug, the candidate drug is not likely to assist in the alleviation of an LQT condition, since the greater current observed in the presence of the candidate drug indicates that the drug might tend to aggravate the LQT condition.

Various statistical operations can be performed in the context of the present method, and all of the methods of the present invention. The circumstances of the experiments, among other conditions, can dictate, in part, the nature of any data analysis that might be performed. For example, if repeated determinations are performed, these data would be more amenable to a statistical analysis than would single determinations. Further, statistics can play a role in assessing the benefit of pursuing further development of a given candidate drug or pharmaceutical. For example, depending on the nature of the statistical analysis performed, conclusions as to whether a given candidate drug is or is not likely to be a HERG channel modulator, treat LQT, etc., can vary. The precise need for, and nature of, any statistical methodology that can be performed in the context of the methods of the present invention will be known to those of skill in the art, upon consideration of the present disclosure.

A voltage clamp assay of the present invention can also comprise determining HERG channel activity in the presence of a test substance and a known HERG channel modulator. For example, the method can comprise: (a) providing an expression system, whereby a functional HERG potassium channel polypeptide and/or a KCR1 polypeptide is expressed; (b) adding a persistent potassium channel activator to the expression system, whereby potassium conductance is elicited; (c) adding a test substance to the expression system; and (d) observing a suppression of the conductance in the presence of the persistent activator and the test substance, whereby an inhibitor of HERG potassium channel polypeptide and/or a KCR1 polypeptide is determined. Optionally, the persistent activator and test substance can be provided to the functional expression simultaneously. Similarly, an assay for determining a HERG potassium channel polypeptide and/or a KCR1 polypeptide activator can comprise steps (a)-(d) above with the exception that an enhancement of conductance is observed in the presence of the persistent activator and the test substance.

VII.B. Conformational Assay

The present invention also provides a method for identifying a KCR1 modulator that relies on a conformational change of a KCR1 polypeptide when bound by or otherwise interacting with a KCR1 modulator.

Application of circular dichroism to solutions of macromolecules reveals the conformational states of these macromolecules. The technique can distinguish random coil, alpha helix, and beta chain conformational states. The secondary structure of a rat sodium channel α-subunit has been determined by circular dichroism as a conformationally flexible polypeptide that contains mostly β-sheet and random coil which fold into a conformation comprising about 65% α-helix (Elmer et al., 1985; Oiki et al., 1990). Provision of a sodium channel antagonist results in a sharp helical transition near body temperature. Addition of a sodium channel agonist alters the temperature-dependent helix transition such that it is observed only at more elevated temperatures. See U.S. Pat. Nos. 5,776,859 and 5,780,242.

To identify modulators of KCR1, circular dichroism analysis can be performed using recombinantly expressed KCR1. KCR1 polypeptide is purified, for example by ion exchange and size exclusion chromatography, and mixed with a test substance. The mixture is subjected to circular dichroism at a wavelength of 222 nM wavelength. The transition of molar ellipticity is compared with a control KCR1 polypeptide that has not been exposed to the test substance. Alpha helical content, as measured at 222 nm, is used to monitor the effect of temperature change on KCR1 conformation. The different conformational state of a KCR1 in the absence of a modulator when compared to a conformational state in the presence of an antagonist, an agonist, or a combination thereof, can thus be used to identify a KCR1 modulator.

VII.C. Binding Assays

In another embodiment, a method for identification of a potassium channel modulator comprises determining specific binding of a test substance to a KCR1 polypeptide. The term “binding” refers to an affinity between two molecules. The term “binding” also encompasses a quality or state of mutual action such that an activity of one protein or compound on another protein is inhibitory (in the case of an antagonist) or enhancing (in the case of an agonist).

The phrase “specifically (or selectively) binds”, when referring to the binding capacity of a candidate modulator, refers to a binding reaction which is determinative of the presence of the protein in a heterogeneous population of proteins and other biological materials. The binding of a modulator to a KCR1 polypeptide can be considered specific if the binding affinity is about 1×10⁴ M⁻¹ to about 1×10⁶ M⁻¹ or greater. The phrase “specifically binds” also refers to saturable binding. To demonstrate saturable binding of a test substance to a KCR1 polypeptide, Scatchard analysis can be carried out as described, for example, by Mak et al. (1989) J Biol Chem 264:21613-21618.

The phases “substantially lack binding” or “substantially no binding”, as used herein to describe binding of a modulator to a control polypeptide or sample, refers to a level of binding that encompasses non-specific or background binding, but does not include specific binding.

Several techniques can be used to detect interactions between a KCR1 polypeptide and a test substance without employing a known competitive modulator. Representative methods include, but are not limited to, Fluorescence Correlation Spectroscopy, Surface-Enhanced Laser Desorption/Ionization Time-Of-flight Spectroscopy, and Biacore technology, each technique described herein below. These methods are amenable to automated, high-throughput screening.

Fluorescence Correlation Spectroscopy.

Fluorescence Correlation Spectroscopy (FCS) measures the average diffusion rate of a fluorescent molecule within a small sample volume. Magde et al., 1972; Maiti et al., 1997. The sample size can be as low as 10³ fluorescent molecules and the sample volume as low as the cytoplasm of a single bacterium. The diffusion rate is a function of the mass of the molecule and decreases as the mass increases. FCS can therefore be applied to polypeptide-ligand interaction analysis by measuring the change in mass and therefore in diffusion rate of a molecule upon binding. In a typical experiment, the target to be analyzed (e.g., a KCR1 polypeptide) is expressed as a recombinant polypeptide with a sequence tag, such as a poly-histidine sequence, inserted at the N-terminus or C-terminus. The expression is mediated in a host cell, such as E. coli, yeast, Xenopus oocytes, or mammalian cells. The polypeptide is purified using chromatographic methods. For example, the poly-histidine tag can be used to bind the expressed polypeptide to a metal chelate column such as Ni²⁺ chelated on iminodiacetic acid agarose. The polypeptide is then labeled with a fluorescent tag such as carboxytetramethylrhodamine or BODIPY™ reagent (available from Molecular Probes of Eugene, Oreg.). The polypeptide is then exposed in solution to the potential ligand, and its diffusion rate is determined by FCS using instrumentation available from Carl Zeiss, Inc. (Thornwood, N.Y.). Ligand binding is determined by changes in the diffusion rate of the polypeptide.

Surface-Enhanced Laser Desorption/Ionization.

Surface-Enhanced Laser Desorption/Ionization (SELDI) was developed by Hutchens & Yip (1993) Rapid Commun Mass Spectrom 7:576-580. When coupled to a time-of-flight mass spectrometer (TOF), SELDI provides a technique to rapidly analyze molecules retained on a chip. It can be applied to ligand-protein interaction analysis by covalently binding the target protein, or portion thereof, on the chip and analyzing by mass spectrometry the small molecules that bind to this protein (Worrall et al., 1998). In a typical experiment, a target polypeptide (e.g., a KCR1 polypeptide) is recombinantly expressed and purified. The target polypeptide is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target polypeptide are identified by the stringency of the wash needed to elute them.

Biacore.

Biacore relies on changes in the refractive index at the surface layer upon binding of a ligand to a target polypeptide (e.g., a KCR1 polypeptide) immobilized on the layer. In this system, a collection of small ligands is injected sequentially in a 2, 3, 4 or 5 microliter cell, wherein the target polypeptide is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al., 1983). In a typical experiment, a target protein is recombinantly expressed, purified, and bound to a Biacore chip. Binding can be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction. See also Homola et al. (1999) Sensors and Actuators 54:3-15 and references therein.

VII.D. Rational Design

The knowledge of the structure a native human KCR1 polypeptide provides an approach for rational design of modulators and diagnostic agents. In brief, the structure of a human KCR1 polypeptide can be determined by X-ray crystallography and/or by computational algorithms that generate three-dimensional representations. See Saqi et al. (1999) Bioinformatics 15:521-522; Huang et al. (2000) Pac Symp Biocomput:230-241; and PCT International Publication No. WO 99/26966. Alternatively, a working model of a human KCR1 polypeptide structure can be derived by homology modeling (Maalouf et al., 1998). Computer models can further predict binding of a protein structure to various substrate molecules that can be synthesized and tested using the assays described herein above. Additional compound design techniques are described in U.S. Pat. Nos. 5,834,228 and 5,872,011.

In general, a KCR1 polypeptide is associated with a membrane protein, i.e. HERG, and can be purified in soluble form using detergents or other suitable amphiphillic molecules. The resulting KCR1 polypeptide is in sufficient purity and concentration for crystallization. The purified and cleaved KCR1 polypeptide preferably runs as a single band under reducing or non-reducing polyacrylamide gel electrophoresis (PAGE). The purified KCR1 polypeptide is can be crystallized under varying conditions of at least one of the following: pH, buffer type, buffer concentration, salt type, polymer type, polymer concentration, other precipitating ligands and concentration of purified and cleaved KCR1. Methods for generation of a crystalline polypeptide are known in the art and can be reasonably adapted for determination of a KCR1 polypeptide as disclosed herein. See e.g., Deisenhofer et al. (1984) J Mol Biol 180:385-398; Weiss et al. (1990) FEBS Lett 267:268-272; or the methods provided in a commercial kit, such as the CRYSTAL SCREEN™ kit (available from Hampton Research of Riverside, Calif.).

A crystallized KCR1 polypeptide is tested for functional activity and differently sized and shaped crystals are further tested for suitability in X-ray diffraction. Generally, larger crystals provide better crystallography than smaller crystals, and thicker crystals provide better crystallography than thinner crystals. Preferably, KCR1 crystals range in size from 0.1-1.5 mm. These crystals diffract X-rays to at least 10 Å resolution, such as 1.5-10.0 Å or any range of value therein, such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3, with 3.5 Å or less being preferred for the highest resolution.

VII.E. Method of Screening for Modulators of In Vivo KCRI Levels

In accordance with the present invention there are also provided methods for screening candidate compounds for the ability to modulate in vivo KCR1 levels. Exemplary modulators of KCR1 levels can thus comprise modulators of KCR1 expression. Pharmaceuticals that increase or decrease the expression of KCR1-encoding genes have clinical application for the treatment or prevention of long QT and other cardiac arrhythmias. The present invention thus includes a method for discovery of compounds that modulate the expression of KCR1-encoding genes and describes the use of such compounds. The general approach is to screen compound libraries for substances that increase or decrease expression of KCR1 encoding genes. Exemplary techniques are described in U.S. Pat. Nos. 5,846,720 and 5,580,722, the entire contents of each of which are herein incorporated by reference.

While the following terms are believed to be well understood by one of skill in the art, the following definitions are set forth to facilitate explanation of the invention.

“Antisense nucleic acid” means an RNA or DNA molecule or a chemically modified RNA or DNA molecule that is complementary to a sequence present within an RNA transcript of a gene.

“Directly transcriptionally modulate the expression of a gene” means to transcriptionally modulate the expression of the gene through the binding of a molecule to (a) the gene, (b) an RNA transcript of the gene, or (c) a protein which binds to (i) such gene or RNA transcripts, or (ii) a protein which binds to such gene or RNA transcript.

A “gene” means a nucleic acid molecule, the sequence of which includes all the information required for the normal regulated production of a particular protein, including the structural coding sequence, promoters and enhancers.

“Indirectly transcriptionally modulate the expression of a gene” means to transcriptionally modulate the expression of such gene through the action of a molecule which cause enzymatic modification of a protein which binds to (a) the gene or (b) an RNA transcript of the gene, or (c) protein which binds to (i) the gene or (ii) an RNA transcript of the gene. For example, altering the activity of a kinase that subsequently phosphorylates and alters the activity of a transcription factor constitutes indirect transcript modulation.

“Ligand” means any binding molecule, and here particularly refers to a molecule that binds to a transcription factor for a gene. The binding of the ligand to the transcription factor transcriptionally modulates the expression of the gene.

“Ligand binding domain of a transcription factor” means the site on the transcription factor at which the ligand binds.

“Modulatable transcriptional regulatory sequence of a gene” means a nucleic acid sequence within the gene to which a transcription factor binds so as to transcriptionally modulate the expression of the gene. Such sequences are identified by any method recognized in the art, including sequencing methods that employ the KCR1 nucleic acids disclosed herein.

“Receptor” means a transcription factor containing a ligand binding domain.

“Specifically transcriptionally modulate the expression of a gene” means to transcriptionally modulate the expression of such gene alone, or together with a limited number of other genes.

“Transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

“Transcription factor for a gene” means a cytoplasmic or nuclear protein which binds to (a) such gene, (b) an RNA transcript of such gene, or (c) a protein which binds to (i) such gene or such RNA transcript or (ii) a protein which binds to such gene or such RNA transcript, so as to thereby transcriptionally modulate expression of the gene.

“Transcriptionally modulate the expression of a gene” means to change the rate of transcription of such gene.

“Triple helix” means a helical structure resulting from the binding of one or more oligonucleotides to double stranded DNA.

In accordance with the present invention there is provided a method of identifying a candidate compound or molecule that is capable of transcriptionally modulating the expression of a gene encoding KCR1, and thus is capable of acting as a therapeutic agent for long QT syndrome. This method comprises contacting a sample that contains a predefined number of cells with a predetermined amount of candidate compound or molecule to be tested. Each such cell comprises DNA comprising (i) a modulatable transcriptional regulatory sequence of a KCR1 gene, (ii) a promoter of a KCR1 gene, and (iii) a DNA sequence encoding a polypeptide other than KCR1, which polypeptide being capable of producing a detectable signal. Thus, the polypeptide can be described as a reporter or marker polypeptide. Preferably, the candidate compound directly and specifically transcriptionally modulates expression of the KCR1-encoding gene.

The DNA sequence is coupled to and under the control of the promoter, under conditions such that the candidate compound or molecule, if capable of acting as a transcriptional modulator of the gene encoding KCR1, causes a measurable detectable signal to be produced by the polypeptide so expressed. This allows for quantitative determination of the amount of the signal produced. By comparing the amount so determined with the amount of produced signal detected in the absence of any molecule being tested or upon contacting the sample with any other molecule, this method allows one to identify the candidate compound or molecule as one which causes a change in the detectable signal produced by the polypeptide so expressed, and thus identifying the molecule as a molecule capable of transcriptionally modulating the expression of the gene encoding KCR1, to thereby identify the candidate compound as a therapeutic agent for, among other things, long QT syndrome.

In the practice of the preceding method the reporter polypeptide may be a luciferase, chloramphenicol acetyltransferase, β-glucuronidase, β-galactosidase, neomycin phosphotransferase, alkaline phosphatase or guanine xanthine phosphoribosyltransferase.

This invention still further provides a method of determining whether a candidate compound or molecule is capable of directly and specifically transcriptionally modulating the expression of a gene encoding KCR1. This method comprises contacting a sample that contains a predefined number of cells with a predetermined amount of a candidate compound or molecule to be tested. Each such cell comprises DNA comprising (i) a modulatable transcriptional regulatory sequence of the gene encoding KCR1, (ii) a promoter of the gene encoding KCR1, and (iii) a reporter gene, which expresses a polypeptide.

The reporter gene is coupled to and under the control of the promoter under conditions such that the candidate compound or molecule, if capable of acting as a transcriptional modulator of the gene encoding KCR1, causes a measurable detectable signal to be produced by the polypeptide so expressed. This allows for quantitative determination of the amount of the signal produced. By comparing the amount so determined with the amount of produced signal detected in the absence of any molecule being tested or upon contacting the sample with any other molecule, this method allows one to identify the candidate compound or molecule as one which causes a change in the detectable signal produced by the polypeptide so expressed, and thus identifying the molecule as a molecule capable of directly and specifically transcriptionally modulating the expression of the gene encoding KCR1, to thereby identify the candidate compound as a therapeutic agent for for, among other things, long QT syndrome.

In the foregoing methods the DNA sequence encoding the polypeptide can be inserted downstream of the promoter of the gene encoding KCR1 by homologous recombination. In certain embodiments of the invention the polypeptide so produced is capable of complexing with an antibody or is capable of complexing with biotin. In this case the resulting complexes can be detected.

Another method of determining whether a candidate compound or molecule is capable of transcriptionally modulating the expression of a gene encoding KCR1 is provided in accordance with the present invention. This method comprises contacting a sample that contains a predefined number of cells with a predetermined amount of a candidate compound or molecule to be tested. Each such cell comprises DNA comprising (i) a modulatable transcriptional regulatory sequence of the gene encoding KCR1, (ii) a promoter of the gene encoding KCR1, and (iii) a DNA sequence transcribable into mRNA coupled to and under the control of the promoter. The contacting is under conditions such that the candidate compound or molecule, if capable of acting as a transcriptional modulator of the protein of interest, causes a measurable difference in the amount of mRNA transcribed from the DNA sequence.

This method allows for the quantitative determination of the amount of the mRNA produced. By comparing the amount so determined with the amount of mRNA detected in the absence of any molecule being tested or upon contacting the sample with any other molecule, one can thereby identify the candidate compound or molecule as one which causes a change in the detectable mRNA amount of, and thus identifying the molecule as a molecule capable of directly and specifically transcriptionally modulating the expression of the gene encoding KCR1. Such a compound is thereby identified as a therapeutic agent for for, among other things, long QT syndrome. The mRNA is optionally detected by quantitative polymerase chain reaction, Northern blot analysis or by any other method as would be apparent to one of skill in the art.

In each of the preceding methods the sample comprises cells in monolayers or cells in suspension. Preferably, such cells are animal cells or human cells. In the presently preferred method the predefined number of cells is from about 1 to about 5×10⁵ cells, or about 2×10² to about 5×10⁴ cells. In these methods the predetermined amount or concentration of the molecule to be tested is typically based upon the volume of the sample, or be from about 1.0 pM to about 20 μM, or from about 10 nM to about 500 μM.

Typically the contacting is effected from about 1 to about 24 hours, preferably from about 2 to about 12 hours. Also the contacting is typically effected with more than one predetermined amount of the molecule to be tested. The molecule to be tested in these methods can be a purified molecule or a homogenous sample. Further, in the method of the invention, the DNA is the cell can comprise, or can consist essentially of, more than one modulatable transcriptional regulatory sequence.

In accordance with the present invention there is also provided a rapid and high throughput screening method that relies on the methods described above. This screening method comprises separately contacting each of a plurality of substantially identical samples, each sample containing a predefined number of cells under conditions such that contacting is affected with a predetermined amount of each different candidate compound or molecule to be tested. In such a screening method the plurality of samples preferably comprises more that about 10⁴ samples, or more preferably comprises more than about 5×10⁴ samples. Also provided is a method of essentially simultaneously screening candidate compounds or molecules to determine whether the molecules are capable of transcriptionally modulating one or more genes encoding KCR1 according to the methods discussed above. These methods are optionally carried out with more than about 10³ samples per week contacted with different candidate compounds or molecules.

VIII. Therapeutic Methods

Given the marked reduction in drug block achieved by KCR1 coexpression in cultured cell systems, KCR1, or subunits of the KCR1 protein, can be employed in a therapeutic approach to preventing the acquired long QT syndrome when drugs are administered to patients. This can be achieved in at least two preferred embodiments:

(1) Directly increase KCR1 expression in the myocardium using a gene therapy approach. Recent studies (e.g. Hoppe, U. C., et al., Proc Natl Acad Sci USA. 98:5335-40 (2001)) have demonstrated the feasibility of directly incorporating ion channels or their subunits into the heart using virus-based approaches, and have proven that these methods can successfully modify the electrophysiologic behavior of the heart. Administration of additional KCR1 to the ventricular myocardium can render HERG less sensitive to drug block; moreover, given that KCR1 has no effect on the baseline functional behavior of HERG, the potential for untoward cardiac side effects is minimal.

(2) Upregulate native cardiac KCR1 expression in reducing I_{Kr} drug block. Particular hormones or other regulators can be administered in order to boost myocardial expression of KCR1, and thereby limit I_{Kr} drug block. Identification and development of such regulators involves an understanding of the expression regulation of KCR1, as is provided in section VII.D. above. This approach provides “combo-drugs”, similar to antibiotic formulations that contain synergistic co-agents (beta lactams+beta lactamase inhibitors). The combined agents facilitate safe administration of drugs that otherwise induce QT prolongation when administered alone.

The present invention thus provides methods for modulation of potassium channel activity in a subject. Modulation can comprise a change in activity of any potassium channel. A preferred method comprises administering to the subject an effective amount of a substance that provides expression of a KCR1-encoding nucleic acid molecule in a cell or tissue where modulated potassium channel function is desired; and modulating potassium channel function in the subject through the administering of the substance. Preferably, the cell or tissue is a cardiac cell or tissue. More preferably, the potassium channel activity that is modulated in a subject comprises an activity of a HERG polypeptide, as defined herein above.

VIII.A. Gene Therapy Approaches

In another embodiment of the invention, a method for modulating potassium channel activity in a subject comprises: (a) preparing a gene therapy vector comprising a nucleotide sequence encoding a KCR1 polypeptide; and (b) administering the gene therapy vector to a subject, whereby the function of a potassium channel in the subject is modulated. The method can further comprise co-administering the gene therapy vector with another therapeutic agent having a different therapeutic effect and having as a side effect the blocking of potassium channel function, preferably HERG function. The combination of agents facilitate safe administration of drugs that otherwise induce QT prolongation when administered alone.

A gene therapy construct of the present invention can comprise: (a) a gene therapy vector; and (b) a nucleic acid molecule encoding a KCR1 polypeptide, wherein the nucleic acid encoding segment is operatively linked to a promoter. Preferably, the KCR1 polypeptide is encoded by a nucleic acid molecule comprising the nucleotide sequence of SEQ ID NO: 1. It is also preferable, but not required, that the potassium channel polypeptide comprise a HERG channel comprising the polypeptide sequence of SEQ ID NO: 3.

A gene therapy construct of the present invention can also comprise: (a) a gene therapy vector; and (b) a nucleic acid molecule encoding a KCR1 polypeptide operatively linked to a promoter. Preferably, a gene therapy construct is prepared as described herein for recombinant expression of a KCR1 polypeptide. Thus, a gene therapy construct of the invention preferably comprises: (a) a nucleotide sequence comprising the nucleotide sequence of SEQ ID NO: 1; or (b) a nucleotide sequence substantially identical to SEQ ID NO: 1.

A gene therapy construct for myocardial expression is described by Hoppe, U. C., et al., Proc Natl Acad Sci USA. 98:5335-40 (2001). Thus, preferably, the gene therapy construct is administered to a cardiac cell or tissue in a subject.

A gene therapy construct for widespread central nervous system expression of a heterologous nucleic acid can employ a platelet-derived growth factor (PDGF) β-chain promoter (Games et al., 1995). For neuron-specific expression, useful promoters include a neuron-specific enolase (NSE) promoter (Forss-Petter et al., 1990; Peel et al., 1997; Klein et al., 1998) and hybrid cytomegalovirus promoters (CMV), for example a CMV/human β-globin hybrid promoter (Mandel et al., 1998) and a CMV/chicken β-actin promoter (Niwa et al., 1991; Dhillon et al., 1999). A glial acidic fibrillary (GFAP) promoter can be used to direct heterologous expression in glia and a subset of neurons (Games et al., 1995). The GFAP promoter is further activated following injury and thus can be useful for gene expression in response to trauma. A myelin basic protein promoter can be used for expression in oligodendrocytes (Ikenaka & Kagawa, 1995; Chen et al., 1998; Chen et al., 1999.

A gene therapy construct of the present invention can also employ an inducible promoter. For example, a tetracycline responsive promoter has been used effectively to regulate transgene expression in rat brain (Mitchell & Habermann, 1999). Other inducible promoters include hormone-inducible promoters (No et al., 1996; Abruzzese et al., 1999; Burcin et al., 1999), radiation-inducible promoters, such as those employing the Egr-1 promoter or NF-_{κ}B promoter (Weichselbaum et al., 1991; Weichselbaum et al., 1994), and heat-inducible promoters (Madio et al., 1998; Gerner et al., 2000; Vekris et al., 2000).

A gene therapy construct can comprise any suitable vector, including but not limited to viruses, plasmids, water-oil emulsions, polyethylene imines, dendrimers, micelles, microcapsules, liposomes, and cationic lipids. Where appropriate, two or more types of vectors can be used together. For example, a plasmid vector can be used in conjunction with liposomes. See e.g., U.S. Pat. No. 5,928,944.

VIII.B. Modulation of KCR1 Levels

A method for transcriptionally modulating in a multicellular organism the expression of a gene encoding KCR1 as in a subject in need thereof is also provided in accordance with the present invention. This method comprises administering to the subject a compound at a concentration effective to transcriptionally modulate expression of KCR1. Preferably, the method elevates levels of KCR1 to thereby treat long QT syndrome. The method can further comprise co-administering the compound with another therapeutic agent having a different therapeutic effect and having as a side effect the blocking of potassium channel function, preferably HERG function. The compound and therapeutic agent can be administered separately or as a formulation comprising both. The combination of agents facilitate safe administration of drugs that otherwise induce QT prolongation when administered alone.

In this method the compound can be identified in accordance with the methods described above and which transcriptionally modulates expression of KCR1. Optionally, the compound directly binds to DNA or RNA, or directly binds to a protein involved in transcription. Thus, indirect and direct transcriptional modulation fall within the scope of the present method.

In an alternative embodiment of the present method the compound does not naturally occur in the cell, specifically transcriptionally modulates expression of the gene encoding the protein of interest, and directly binds to DNA or RNA, or directly binds to a protein at a site on such protein which is not a ligand-binding domain of a receptor which naturally occurs in the cell. Preferably, the cell contacted in accordance with this method is a human cell.

Preferred chemical entities do not naturally occur in any cell of a lower eukaryotic organism such as yeast. More preferably, chemical entities do not naturally occur in any cell, whether of a multicellular or a unicellular organism. Even more preferably, the chemical entity is not a naturally occurring molecule, e.g. it is a chemically synthesized entity.

Optionally, the compound can bind to a modulatable transcription sequence of the gene. For example, the compound can bind to a promoter region upstream of a nucleic acid sequence encoding KCR1. In the methods above, modulation of the transcription of KCR1 results in either upregulation or downregulation of expression of the gene encoding the protein of interest, depending on the identity of the molecule which contacts the cell. Preferably, the method elevates levels of KCR1 by activating expression of KCR1, and this embodiment can be employed in the treatment of long QT syndrome.

It is also provided according to the present invention that expression of KCR1 can be modulated in the vertebrate subject through the administration of an antisense oligonucleotide derived from a nucleic acid molecule encoding KCR1, e.g. SEQ ID NO: 1. Therapeutic methods utilizing antisense oligonucleotides have been described in the art, for example, in U.S. Pat. Nos. 5,627,158 and 5,734,033, the contents of each of which are herein incorporated by reference.

In one embodiment of the methods of the invention above the compound comprises an antisense nucleic acid that is complementary to a sequence present in a modulatable, transcriptional sequence. The compound can also be a double-stranded nucleic acid or a nucleic acid capable of forming a triple helix with a double-stranded DNA.

VIII.C. Modulation of KCR1 and/or HERG Activity

KCR1 and/HERG modulators identified using the compositions and methods disclosed herein above can also be used in the treatment of potassium channel-related disorders, e.g. long QT syndrome. Preferably, KCR1 modulators display a biological activity including but not limited to modulating potassium ion flow, modulating cardiac rhythms (including reversing or preventing long QT syndrome), and combinations thereof, as described herein below.

In one embodiment of the invention, a method for modulating potassium channel function in a subject comprises: (a) preparing a composition, comprising a modulator identified according to the methods disclosed herein above, and a pharmaceutically acceptable carrier; (b) administering an effective dose of the composition to a subject, whereby potassium channel activity is altered in the subject. The method can further comprise co-administering the compound with another therapeutic agent having a different therapeutic effect and having as a side effect the blocking of potassium channel function, preferably HERG function. The compound and therapeutic agent can be administered separately or as a formulation comprising both. The combination of agents facilitate safe administration of drugs that otherwise induce QT prolongation when administered alone.

VIII.D. Preparation of a Composition

The present invention also provides a method for preparing a composition comprising a KCR1 modulator or a recombinantly expressed KCR1 polypeptide. Such a composition can comprise a drug carrier and can be formulated in any manner suitable for administration to a subject. Optionally, the composition can further comprise a targeting ligand to facilitate delivery to a site in need of treatment.

Drug Carriers.

Any suitable drug delivery vehicle or carrier can be used, including but not limited to a gene therapy vector (e.g., a viral vector or a plasmid), a microcapsule, for example a microsphere (U.S. Pat. Nos. 5,871,778 and 5,690,954) or a nanosphere (U.S. Pat. Nos. 6,207,195 and 6,177088), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate, and U.S. Pat. Nos. 4,551,482, 5,714,166, 5,510,103, 5,490,840, and 5,855,900), and a polysome (U.S. Pat. No. 5,922,545).

Targeting Ligands.

The term “target cell” as used herein refers to a cell intended to be treated by a therapeutic agent. A target cell is preferably a cell in a subject in need of therapeutic treatment. For example, a target cell can comprise a cell having abnormal potassium channel activity.

As desired, compositions of the present invention can include a targeting or homing molecule that facilitates delivery of a drug comprising a KCR1 modulator to an intended in vivo site. A targeting molecule can comprise, for example, a ligand that shows specific affinity for a target molecule in the target tissue. A targeting molecule can also comprise a structural design that mediates tissue-specific localization.

Antibodies, peptides, or other ligands can be coupled to drugs or drug carriers using methods known in the art, including but not limited to carbodiimide conjugation, esterification, sodium periodate oxidation followed by reductive alkylation, and glutaraldehyde crosslinking. See Goldman et al. (1997) Cancer Res 57:1447-1451; Cheng (1996) Hum Gene Ther 7:275-282; Neri et al. (1997) Nat Biotechnol 15:1271-1275; Nabel (1997), Current Protocols in Human Genetics. John Wiley & Sons, New York, Vol. on CD-ROM; Park et al. (1997) Adv Pharmacol 40:399-435; Pasqualini et al. (1997) Nat Biotechnol 15:542-546; Bauminger & Wilchek (1980) Methods Enzymol 70:151-159; U.S. Pat. No. 6,071,890; and European Patent No. 0 439 095.

Formulation.

A composition of the present invention preferably comprises a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some preferred ingredients are sodium dodecyl sulfate (SDS), for example in the range of about 0.1 to about 10 mg/ml, preferably about 2.0 mg/ml; and/or mannitol or another sugar, for example in the range of 10 to 100 mg/ml, preferably about 30 mg/ml; and/or phosphate-buffered saline (PBS). Any other agents conventional in the art having regard to the type of formulation in question can be used.

Administration.

Suitable methods for administering a drug of the present invention to a subject include but are not limited to systemic administration, parenteral administration (including intravascular, intramuscular, intraarterial administration), oral delivery, subcutaneous administration, inhalation, intratracheal installation, surgical implantation, transdermal delivery, local injection, and hyper-velocity injection/bombardment. Where applicable, continuous infusion can enhance drug accumulation at a target site (e.g., U.S. Pat. No. 6,180,082).

The particular mode of drug administration of the present invention depends on various factors, including but not limited to the vector and/or drug carrier employed, the severity of the condition, and mechanisms for metabolism or removal of the drug from its site of administration.

The administration method can further include treatments for enhancing drug delivery. Representative methods include ionotophoresis (U.S. Pat. No. 6,001,088; 5,499,971), electroporation (U.S. Pat. No. 6,041,253), electromagnetic field generation by ultra-wide band short pulses (U.S. Pat. No. 6,261,831), and hormone treatment (U.S. Pat. No. 5,962,667).

The administration method can also include treatments for drug release or drug activation. For example, a composition comprising a therapeutic agent conjugated to a drug carrier or targeting molecule via a selectively hydrolyzable bond can be released by local provision of a hydrolyzing agent (U.S. Pat. No. 5,762,918). In the case of a gene therapy construct, gene expression of a therapeutic polypeptide or therapeutic oligonucleotide can be regulated using an inducible promoter. Thus an administration method can further comprise a method for induction of a gene therapy construct.

The administration method employed can include any treatment that augments drug efficacy.

Dose.

For therapeutic applications, a therapeutically effective amount of a composition of the invention is administered to a subject. A “therapeutically effective amount” is an amount of the therapeutic composition sufficient to produce a measurable biological response (for example, but not limited to, a change in potassium ion current, modulating cardiac rhythms (including reversing or preventing long QT syndrome, and the like). Actual dosage levels of active ingredients in a therapeutic composition of the invention can be varied so as to administer an amount of the active compound(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For administration of therapeutic compositions comprising a small molecule, conventional methods of extrapolating human dosage based on doses administered to a murine animal model can be carried out using the conversion factor for converting the mouse dosage to human dosage: Dose Human per kg=Dose Mouse per kg×12 (Freireich et al. (1966) Cancer Chemother Rep 50:219-244). Drug doses can also given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (1966) Cancer Chemother Rep 50:219-244. Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/sq m. See also U.S. Pat. Nos. 5,326,902 and 5,234,933, and PCT International Publication No. WO 93/25521.

For local administration of viral vectors, previous clinical studies have demonstrated that up to 10¹³ pfu of virus can be injected with minimal toxicity. In human patients, 1×10⁹-1×10¹³ pfu are routinely used. See Habib et al. (1999) Human Gene Therapy 12:2019-2034. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize KCR1 expression.

For additional guidance regarding dose, see Berkow et al. (1997) The Merck Manual of Medical Information, Home ed. Merck Research Laboratories, Whitehouse Station, N.J.; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla.; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; Speight et al. (1997) Avery's Drug Treatment: A Guide to the Properties, Choice, Therapeutic Use and Economic Value of Drugs in Disease Management, 4th ed. Adis International, Auckland/Philadelphia; Duch et al. (1998) Toxicol Lett 100-101:255-263.

IX. KCR1 Polymorphisms

Relatively common gene sequence variations (known as “polymorphisms”) have been identified in the coding regions of HERG and HERG-associated proteins (such as MiRP1) that influence the likelihood that drugs will block I_{Kr} current, and thus induce ECG QT interval prolongation and the Torsades de Pointes arrhythmia. Abbott, G. W., et al., Cell 97:175-87(1999); Sesti, F., et al., Proc Natl Acad Sci USA. 97:10613-8 (2000).

As disclosed in Laboratory Example 5 below, given the evidence provided that KCR1 also modulates the blockade of HERG and I_{Kr} by drugs disclosed herein above, a database of DNA from acquired long QT patients collected at Vanderbilt University was examined. It was observed that the KCR1 polymorphism I447V is present at an allele frequency of 1.1%. This allele is significantly more common (7%, p<0.05 by Chi-Square analysis) in a control database of randomly selected individuals with ethnicities representing the Middle Tennessee area. Hence, it is envisioned that I447V is a risk-lowering allele in KCR1, which further provides that KCR1 is a screening target for gene sequence variations that raise or lower the risk of acquired long QT syndrome during drug therapy.

IX.A. Polynucleotide Screening Methods

In accordance with the present invention, a method of screening for susceptibility to drug-induced cardiac arrhythmias in a subject is provided. The method comprising: (a) obtaining a nucleic acid sample from the subject; and (b) detecting a polymorphism of a KCR1 gene in the nucleic acid sample from the subject, the presence of the polymorphism indicating that the susceptibility of the subject to drug-induced cardiac arrhythmias.

As used herein and in the claims, the term “susceptibility” collective refers to both a higher and a lower susceptibility to drug-induced cardiac arrhythmias. Thus, subjects that face a higher or a lower risk of suffering a drug induced cardiac arrhythmia can be identified in accordance with the present invention.

As used herein and in the claims, the term “polymorphism” refers to the occurrence of two or more genetically determined alternative sequences or alleles in a population. A polymorphic marker is the locus at which divergence occurs. Preferred markers have at least two alleles, each occurring at frequency of greater than 1%. A polymorphic locus can be as small as one base pair.

As used herein and in the claims, the term “gene” is used for simplicity to refer to a functional protein, polypeptide or peptide encoding unit. As will be understood by those in the art, this functional term includes both genomic sequences and cDNA sequences. “Isolated substantially away from other coding sequences” means that the gene of interest, in this case, the KCR1 gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

Useful nucleic acid molecules according to the present invention include those that will specifically hybridize to KCR1 sequences in the region of an A to G transition at nucleotide 1339 that leads to the I447V change in the encoded KCR1 polypeptide. Typically these are at least about 20 nucleotides in length and have the nucleotide sequence corresponding to the region of an A to G transition at nucleotide 1339 of a consensus KCR1 cDNA sequence. The term “consensus sequence”, as used herein, is meant to refer to a nucleic acid or protein sequence for KCR1, the nucleic or amino acids of which are known to occur with high frequency in a population of individuals who carry the gene which codes for a normally functioning protein, or which nucleic acid itself has normal function.

Provided nucleic acid molecules can be labeled according to any technique known in the art, such as with radiolabels, fluorescent labels, enzymatic labels, sequence tags, etc. According to another aspect of the invention, the nucleic acid molecules contain the A to G transition at nucleotide 1339 of SEQ ID NO: 1. Such molecules can be used as allele-specific oligonucleotide probes.

Body samples can be tested to determine whether the KCR1 gene contains a polymorphism, such as the I447V polymorphism. Suitable body samples for testing include those comprising DNA, RNA or protein obtained from biopsies, including liver and intestinal tissue biopsies; or from blood, prenatal; or embryonic tissues, for example.

In one embodiment of the invention two pairs of isolated oligonucleotide primers are provided as set forth in the Examples below. These sets of primers are optionally derived from the KCR1 single exon, for example, the location of the KCR1-I447V polymorphism. The oligonucleotide primers are useful in diagnosis of a subject at risk for developing drug-induced cardiac arrhythmias. The primers direct amplification of a target polynucleotide prior to sequencing. These unique KCR1 exon oligonucleotide primers are designed and produced based upon the A to G transition at nucleotide 1339 associated with the KCR1-I447V polymorphism, or based on any other KCR1 polymorphism.

In another embodiment of the invention isolated allele specific oligonucleotides (ASO) are provided. Sequences substantially similar thereto are also provided in accordance with the present invention. The ASOs are useful in diagnosis of a subject at risk developing drug-induced cardiac arrhythmias. These unique KCR1 exon oligonucleotide primers are designed and produced based upon the A to G transition at nucleotide 1339 associated with the KCR1-I447V polymorphism, or based on any other KCR1 polymorphism.

The terms “substantially complementary to” or “substantially the sequence of” refer to sequences which hybridize to the sequences provided (e.g. SEQ ID NO: 1) under stringent conditions as disclosed herein and/or sequences having sufficient homology with SEQ ID NO: 1, such that the allele specific oligonucleotides of the invention hybridize to the sequence. The term “isolated” as used herein includes oligonucleotides substantially free of other nucleic acids, proteins, lipids, carbohydrates or other materials with which they can be associated, such association being either in cellular material or in a synthesis medium. A “target polynucleotide” or “target nucleic acid” refers to the nucleic acid sequence of interest e.g., a KCR1-encoding KCR1 polynucleotide. Other primers that can be used for primer hybridization are readily ascertainable to those of skill in the art based upon the disclosure herein of the KCR1-I447V polymorphism and its association with a lowered risk of drug-induced cardiac arrhythmias, or based on any other KCR1 polymorphism.

The primers of the invention embrace oligonucleotides of sufficient length and appropriate sequence so as to provide initiation of polymerization on a significant number of nucleic acids in the polymorphic locus. Specifically, the term “primer” as used herein refers to a sequence comprising two or more deoxyribonucleotides or ribonucleotides, preferably more than three, and more preferably more than eight and most preferably at least about 20 nucleotides of the KCR1 gene. Preferably, the DNA sequence contains the A to G transition at nucleotide 1339 relative to KCR1 as set forth in SEQ ID NO: 1. The allele including A at base 1339 relative to KCR1 as set forth in SEQ ID NO: 1 is referred to herein as the “KCR1a allele”, the “I447 allele”, or the “isoleucine-encoding allele”. The allele including G at base 1339 relative to KCR1 as set forth in SEQ ID NO: 1 is referred to herein as the “KCR1 b allele”, the “V447 allele”, or the “valine-encoding allele”.

An oligonucleotide that distinguishes between the KCR1a and the KCR1b alleles of the KCR1 gene, wherein said oligonucleotide hybridizes to a portion of the KCR1 gene that includes nucleotide 1339 of a cDNA that corresponds to the KCR1 gene when said nucleotide 1339 is G, but does not hybridize with said portion of said KCR1 gene when said nucleotide 1339 is A is also provided in accordance with the present invention. An oligonucleotide that distinguishes between the KCR1a and the KCR1b alleles of the KCR1 gene, wherein said oligonucleotide hybridizes to a portion of the KCR1 gene that includes nucleotide 1339 of the cDNA that corresponds to the KCR1 gene when nucleotide 1339 is A, but does not hybridize with the portion of the KCR1 gene when nucleotide 1339 is G, is also provided in accordance with the present invention. Such oligonucleotides are preferably between ten and thirty bases in length. Such oligonucleotides can optionally further comprises a detectable label.

Environmental conditions conducive to synthesis include the presence of nucleoside triphosphates and an agent for polymerization, such as DNA polymerase, and a suitable temperature and pH. The primer is preferably single stranded for maximum efficiency in amplification, but can be double stranded. If double stranded, the primer is first treated to separate its strands before being used to prepare extension products. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it can contain fewer nucleotides.

Primers of the invention are designed to be “substantially” complementary to each strand of the genomic locus to be amplified. This means that the primers must be sufficiently complementary to hybridize with their respective strands under conditions that allow the agent for polymerization to perform. In other words, the primers should have sufficient complementarity with the 5′ and 3′ sequences flanking the transition to hybridize therewith and permit amplification of the genomic locus.

Oligonucleotide primers of the invention are employed in the amplification method, which is an enzymatic chain reaction that produces exponential quantities of polymorphic locus relative to the number of reaction steps involved. Typically, one primer is complementary to the negative (−) strand of the polymorphic locus and the other is complementary to the positive (+) strand. Annealing the primers to denatured nucleic acid followed by extension with an enzyme, such as the large fragment of DNA polymerase I (Klenow) and nucleotides, results in newly synthesized + and − strands containing the target polymorphic locus sequence. Because these newly synthesized sequences are also templates, repeated cycles of denaturing, primer annealing, and extension results in exponential production of the region (i.e., the target polymorphic locus sequence) defined by the primers. The product of the chain reaction is a discreet nucleic acid duplex with termini corresponding to the ends of the specific primers employed.

The oligonucleotide primers of the invention can be prepared using any suitable method, such as conventional phosphotriester and phosphodiester methods or automated embodiments thereof. In one such automated embodiment, diethylphosphoramidites are used as starting materials and can be synthesized as described by Beaucage et al., Tetrahedron Letters 22:1859-1862 (1981). One method for synthesizing oligonucleotides on a modified solid support is described in U.S. Pat. No. 4,458,066.

Any nucleic acid specimen, in purified or non-purified form, can be utilized as the starting nucleic acid or acids, providing it contains, or is suspected of containing, a nucleic acid sequence containing the polymorphic locus. Thus, the method can amplify, for example, DNA or RNA, including messenger RNA, wherein DNA or RNA can be single stranded or double stranded. In the event that RNA is to be used as a template, enzymes, and/or conditions optimal for reverse transcribing the template to DNA would be utilized. In addition, a DNA-RNA hybrid that contains one strand of each can be utilized. A mixture of nucleic acids can also be employed, or the nucleic acids produced in a previous amplification reaction herein, using the same or different primers can be so utilized. The specific nucleic acid sequence to be amplified, i.e., the polymorphic locus, can be a fraction of a larger molecule or can be present initially as a discrete molecule, so that the specific sequence constitutes the entire nucleic acid. It is not necessary that the sequence to be amplified be present initially in a pure form; it can be a minor fraction of a complex mixture, such as contained in whole human DNA.

DNA utilized herein can be extracted from a body sample, such as blood, tissue material (e.g. cardiac tissue), and the like by a variety of techniques such as that described by Maniatis et. al. in Molecular Cloning: A Laboratory Manual, Cold Spring Harbor, N.Y., p 280-281 (1982). If the extracted sample is impure, it can be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP, and dTTP are added to the synthesis mixture, either separately or together with the primers, in adequate amounts and the resulting solution is heated to about 90-100° C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization can also be added together with the other reagents if it is heat stable. This synthesis (or amplification) reaction can occur at room temperature up to a temperature above which the agent for polymerization no longer functions. Thus, for example, if DNA polymerase is used as the agent, the temperature is generally no greater than about 40° C. Most conveniently the reaction occurs at room temperature.

The agent for polymerization can be any compound or system that will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (i.e., those enzymes which perform primer extension after being subjected to temperatures sufficiently elevated to cause denaturation), such as Taq polymerase. Suitable enzyme will facilitate combination of the nucleotides in the proper manner to form the primer extension products that are complementary to each polymorphic locus nucleic acid strand. Generally, the synthesis will be initiated at the 3′ end of each primer and proceed in the 5′ direction along the template strand, until synthesis terminates, producing molecules of different lengths.

The newly synthesized strand and its complementary nucleic acid strand will form a double-stranded molecule under hybridizing conditions described herein and this hybrid is used in subsequent steps of the method. In the next step, the newly synthesized double-stranded molecule is subjected to denaturing conditions using any of the procedures described above to provide single-stranded molecules.

The steps of denaturing, annealing, and extension product synthesis can be repeated as often as needed to amplify the target polymorphic locus nucleic acid sequence to the extent necessary for detection. The amount of the specific nucleic acid sequence produced will accumulate in an exponential fashion. PCR. A Practical Approach, ILR Press, Eds. McPherson et al. (1992).

The amplification products can be detected by Southern blot analysis with or without using radioactive probes. In one such method, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation.

Sequences amplified by the methods of the invention can be further evaluated, detected, cloned, sequenced, and the like, either in solution or after binding to a solid support, by any method usually applied to the detection of a specific DNA sequence such as dideoxy sequencing, PCR, oligomer restriction (Saiki et al., Bio/Technology 3:1008-1012 (1985), allele-specific oligonucleotide (ASO) probe analysis (Conner et al., Proc. Natl. Acad. Sci. U.S.A. 80:278 (1983), oligonucleotide ligation assays (OLAs) (Landgren et. al., Science 241:1007, 1988), and the like. Molecular techniques for DNA analysis have been reviewed (Landgren et. al., Science 242:229-237 (1988)).

Preferably, the method of amplifying is by PCR, as described herein and in U.S. Pat. Nos. 4,683,195; 4,683,202; and 4,965,188 each of which is hereby incorporated by reference; and as is commonly used by those of ordinary skill in the art. Alternative methods of amplification have been described and can also be employed as long as the KCR1 locus amplified by PCR using primers of the invention is similarly amplified by the alternative techniques. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA.

Another nucleic acid amplification technique is nucleic acid sequence-based amplification (NASBA™) which uses reverse transcription and T7 RNA polymerase and incorporates two primers to target its cycling scheme. NASBA™ amplification can begin with either DNA or RNA and finish with either, and amplifies to about 10⁸ copies within 60 to 90 minutes.

Alternatively, nucleic acid can be amplified by ligation-activated transcription (LAT). LAT works from a single-stranded template with a single primer that is partially single-stranded and partially double-stranded. Amplification is initiated by ligating a cDNA to the promoter olignucleotide and within a few hours, amplification is about 10⁸ to about 10⁹ fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest.

Another nucleic acid amplification technique, ligase chain reaction (LCR), works by using two differently labeled halves of a sequence of interest, which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligo probe pairs, and the RCR fills and joins the gap, mimicking normal DNA repair.

Nucleic acid amplification by strand displacement activation (SDA) utilizes a short primer containing a recognition site for Hincll with short overhang on the 5′ end, which binds to target DNA. A DNA polymerase fills in the part of the primer opposite the overhang with sulfur-containing adenine analogs. Hincll is added but only cuts the unmodified DNA strand. A DNA polymerase that lacks 5′ exonuclease activity enters at the site of the nick and begins to polymerize, displacing the initial primer strand downstream and building a new one which serves as more primer.

SDA produces greater than about a 10⁷-fold amplification in 2 hours at 37° C. Unlike PCR and LCR, SDA does not require instrumented temperature cycling. Another amplification system useful in the method of the invention is the QB Replicase System. Although PCR is the preferred method of amplification if the invention, these other methods can also be used to amplify the KCR1 locus as described in the method of the invention. Thus, the term “amplification technique” as used herein and in the claims is meant to encompass all the foregoing methods.

In another embodiment of the invention a method is provided for diagnosing or identifying a subject having a lower or higher susceptibility to developing drug-induced cardiac arrhythmias, comprising sequencing a target nucleic acid of a sample from a subject by dideoxy sequencing, preferably following amplification of the target nucleic acid, to identify a KCR1 polymorphism.

In another embodiment of the invention a method is provided for diagnosing a subject having a lower or higher susceptibility to developing drug-induced cardiac arrhythmias, comprising contacting a target nucleic acid of a sample from a subject with a reagent that detects the presence of a KCR1 polymorphism and detecting the reagent.

Another method comprises contacting a target nucleic acid of a sample from a subject with a reagent that detects the presence of an A to G transition at nucleotide 1339 associated with the KCR1-I447V polymorphism, and detecting the transition. A number of hybridization methods are well known to those skilled in the art. Many of them are useful in carrying out the invention.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those of ordinary skill in the art. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. See e.g. Wetmur & Davidson, J. Mol. Biol. 31:349-370 (1968)).

Accordingly, a nucleotide sequence of the present invention can be used for its ability to selectively form duplex molecules with complementary stretches of the KCR1 gene. Depending on the application envisioned, one employs varying conditions of hybridization to achieve varying degrees of selectivity of the probe toward the target sequence. For applications requiring a high degree of selectivity, one typically employs relatively stringent conditions to form the hybrids. For example, one selects relatively low salt and/or high temperature conditions, such as provided by 0.02M-0.15M salt at temperatures of about 50° C. to about 70° C. including particularly temperatures of about 55° C., about 60° C. and about 65° C. Such conditions are particularly selective, and tolerate little, if any, mismatch between the probe and the template or target strand.

Of course, for some applications, for example, where one desires to prepare mutants employing a mutant primer strand hybridized to an underlying template or where one seeks to isolate polypeptide coding sequences from related species, functional equivalents, or the like, less stringent hybridization conditions are typically needed to allow formation of the heteroduplex. Under such circumstances, one employs conditions such as 0.15M-0.9M salt, at temperatures ranging from about 20° C. to about 55° C., including particularly temperatures of about 25° C., about 37° C., about 45° C., and about 50° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results. Other hybridization conditions are described elsewhere herein.

In certain embodiments, it is advantageous to employ a nucleic acid sequence of the present invention in combination with an appropriate reagent, such as a label, for determining hybridization. A wide variety of appropriate indicator reagents are known in the art, including radioactive, enzymatic or other ligands, such as avidin/biotin, which are capable of giving a detectable signal. In preferred embodiments, one likely employs an enzyme tag such a urease, alkaline phosphatase or peroxidase, instead of radioactive or other environmentally undesirable reagents. In the case of enzyme tags, calorimetric indicator substrates are known which can be employed to provide a reagent visible to the human eye or spectrophotometrically, to identify specific hybridization with complementary nucleic acid-containing samples.

In general, it is envisioned that the hybridization probes described herein are useful both as reagents in solution hybridization as well as in embodiments employing a solid phase. In embodiments involving a solid phase, the sample containing test DNA (or RNA) is adsorbed or otherwise affixed to a selected matrix or surface. This fixed, single-stranded nucleic acid is then subjected to specific hybridization with selected probes under desired conditions. The selected conditions depend inter alia on the particular circumstances based on the particular criteria required (depending, for example, on the G+C contents, type of target nucleic acid, source of nucleic acid, size of hybridization probe, etc.). Following washing of the hybridized surface so as to remove nonspecifically bound probe molecules, specific hybridization is detected, or even quantified, via the label.

The materials for use in the method of the invention are ideally suited for the preparation of a screening kit. Such a kit can comprise a carrier having compartments to receive in close confinement one or more containers such as vials, tubes, and the like, each of the containers comprising one of the separate elements to be used in the method. For example, one of the containers can comprise an amplifying reagent for amplifying KCR1 DNA, such as the necessary enzyme(s) and oligonucleotide primers for amplifying target DNA from the subject.

A kit in accordance with the present invention can further comprise solutions, buffers or other reagents for extracting a nucleic acid sample from a biological sample obtained from a subject. Any such reagents as would be readily apparent to one of ordinary skill in the art are within the scope of the present invention. By way of particular example, a suitable lysis buffer for the tissue or cells along with a suspension of glass beads for capturing the nucleic acid sample and an elution buffer for eluting the nucleic acid sample off of the glass beads comprise a reagent for extracting a nucleic acid sample from a biological sample obtained from a subject.

Other examples include commercially available extraction kits, such as the GENOMIC ISOLATION KIT A.S.A.P.™ (Boehringer Mannheim, Indianapolis, Ind.), Genomic DNA Isolation System (GIBCO BRL, Gaithersburg, Md.), ELU-QUIK™ DNA Purification Kit (Schleicher & Schuell, Keene, N.H.), DNA Extraction Kit (Stratagene, La Jolla, Calif.), TURBOGEN™ Isolation Kit (Invitrogen, San Diego, Calif.), and the like. Use of these kits according to the manufacturer's instructions is generally acceptable for purification of DNA prior to practicing the methods of the present invention.

IX.B. Polypeptide/Antibody Screening Methods

In another embodiment, the present invention provides an antibody immunoreactive with a KCR1 polypeptide or KCR1 polynucleotide. Preferably, an antibody of the invention is a monoclonal antibody. Techniques for preparing and characterizing antibodies are well known in the art (See e.g. Antibodies A Laboratory Manual, E. Howell and D. Lane, Cold Spring Harbor Laboratory, 1988). More preferred antibodies distinguish between the different forms of the KCR1 polypeptide (e.g., a polypeptide encoded by the nucleic acid sequence of SEQ ID NO: 1), which comprise the KCR1-I447V polymorphism.

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide can vary in its immunogenicity. It is often necessary therefore to couple the immunogen (e.g., a polypeptide or polynucleotide) of the present invention) with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Reagents for conjugating a polypeptide or a polynucleotide to a carrier protein are well known in the art and include glutaraldehyde, m-maleimidobencoyl-N-hydroxysuccinimide ester, carbodiimide and bis-biazotized benzidine. As is also well known in the art, immunogencity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used of the production of polyclonal antibodies varies, inter alia, upon the nature of the immunogen as well as the animal used for immunization. A variety of routes can be used to administer the immunogen, e.g. subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

Thus, in one aspect, the present invention provides a method of producing an antibody immunoreactive with a KCR1 polypeptide encoded by a KCR1 gene, the method comprising: (a) transfecting recombinant host cells with a KCR1 polynucleotide that encodes the KCR1 polypeptide; (b) culturing the host cells under conditions sufficient for expression of the polypeptide; (c) recovering the polypeptide; and (d) preparing antibodies to the polypeptide. The present invention also provides antibodies prepared according to the method described above.

A monoclonal antibody of the present invention can be readily prepared through use of well-known techniques such as those exemplified in U.S. Pat. No. 4,196,265, herein incorporated by reference. Typically, a technique involves first immunizing a suitable animal with a selected antigen (e.g., a KCR1 polypeptide or KCR1 polynucleotide) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with antigen-polypeptides. The selected clones can then be propagated indefinitely to provide the monoclonal antibody.

By way of specific example, to produce an antibody of the present invention, mice are injected intraperitoneally with between about 1-200 μg of an antigen comprising a KCR1 polypeptide. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/KCR1 polypeptide. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/KCR1 polypeptide. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific KCR1 polypeptides and KCR1 polynucleotides can be recognized as antigens, and thus identified. Once identified, those polypeptides and polynucleotides can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

The present invention thus also provides a method of screening a biological sample for the presence of a KCR1 polypeptide encoded by a KCR1 polynucleotide. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide. Cardiac tissues comprise tissues of particular interest.

Preferably, antibodies that distinguish between the I447 KCR1 polypeptide and the V447 KCR1 polypeptide are provided. Such antibodies can comprise polyclonal antibodies but are preferably monoclonal antibodies prepared as described hereinabove.

In accordance with a screening assay method, a biological sample is exposed to an antibody immunoreactive with the polypeptide whose presence is being assayed. Typically, exposure is accomplished by forming an admixture in a liquid medium that contains both the antibody and the candidate polypeptide. Either the antibody or the sample with the polypeptide can be affixed to a solid support (e.g., a column or a microtiter plate).

The biological sample is exposed to the antibody under biological reaction conditions and for a period of time sufficient for antibody-polypeptide conjugate formation. Biological reaction conditions include ionic composition and concentration, temperature, pH and the like. Ionic composition and concentration can range from that of distilled water to a 2 molal solution of NaCl. Preferably, osmolality is from about 100 mosmols/l to about 400 mosmols/l and, more preferably from about 200 mosmols/l to about 300 mosmols/l. Temperature preferably is from about 4° C. to about 100° C., more preferably from about 15° C. to about 500C and, even more preferably from about 25° C. to about 40° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for antibody-polypeptide conjugate formation and that the conditions do not adversely affect either the antibody or the polypeptide.

Exposure time will vary inter alia with the biological conditions used, the concentration of antibody and polypeptide and the nature of the sample (e.g., fluid or tissue sample). Techniques for determining exposure time are well known to one of ordinary skill in the art. Typically, where the sample is fluid and the concentration of polypeptide in that sample is about 10⁻¹⁰M, exposure time is from about 10 minutes to about 200 minutes.

The presence of polypeptide in the sample is detected by detecting the formation and presence of antibody-polypeptide conjugates. Techniques for detecting such antibody-antigen (e.g., KCR1 polypeptide) conjugates or complexes are well known in the art and include such procedures as centrifugation, affinity chromatography and the like, binding of a secondary antibody to the antibody-candidate receptor complex.

In one embodiment, detection is accomplished by detecting an indicator affixed to the antibody. Exemplary and well known such indicators include radioactive labels (e.g., ³²P, ¹²⁵I, ¹⁴C), a second antibody or an enzyme such as horseradish peroxidase. Techniques for affixing indicators to antibodies are well known in the art. Commercial kits are available.

In another aspect, the present invention provides a method of screening a biological sample for the presence of antibodies immunoreactive with a KCR1 polypeptide encoded by a KCR1 polynucleotide. In accordance with such a method, a biological sample is exposed to a KCR1 polypeptide under biological conditions and for a period of time sufficient for antibody-polypeptide conjugate formation and the formed conjugates are detected.

In another aspect, the present invention provides screening assay kits for detecting the presence of a KCR1 polypeptide encoded by a KCR1 polynucleotide in biological samples, where the kits comprise a first container containing a first antibody capable of immunoreacting with the polypeptide, with the first antibody present in an amount sufficient to perform at least one assay. Preferably, the assay kits of the invention further comprise a second container containing a second antibody that immunoreacts with the first antibody. More preferably, the antibodies used in the assay kits of the present invention are monoclonal antibodies. Even more preferably, the first antibody is affixed to a solid support. More preferably still, the first and second antibodies comprise an indicator, and, preferably, the indicator is a radioactive label or an enzyme.

In another aspect, the present invention provides screening assay kits for detecting the presence, in a biological sample, of antibodies immunoreactive with a KCR1 polypeptide encoded by a KCR1 polynucleotide, the kits comprising a first container containing a KCR1 polypeptide that immunoreacts with the antibodies, with the polypeptide present in an amount sufficient to perform at least one assay. The reagents of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media or a microscope slide. When the reagent provided is a dry powder, the powder can be reconstituted by the addition of a suitable solvent. The solvent can be provided.

Summarily, the detection and screening assays disclosed herein are used as a part of a screening method. Human KCR1-encoding polynucleotides as well as their protein products can be readily used in clinical setting to screen for and to diagnose susceptibility to drug-induced cardiac arrhythmias in humans.

### LABORATORY EXAMPLES

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Methods for Laboratory Examples 1 to 4

The following methods were employed in Laboratory Examples 1 to 4. Laboratory Examples 1 to 4 are discussed immediately following the presentation of the Methods section.

### Identification of Human KCR1 Sequence and Northern Anaylsis

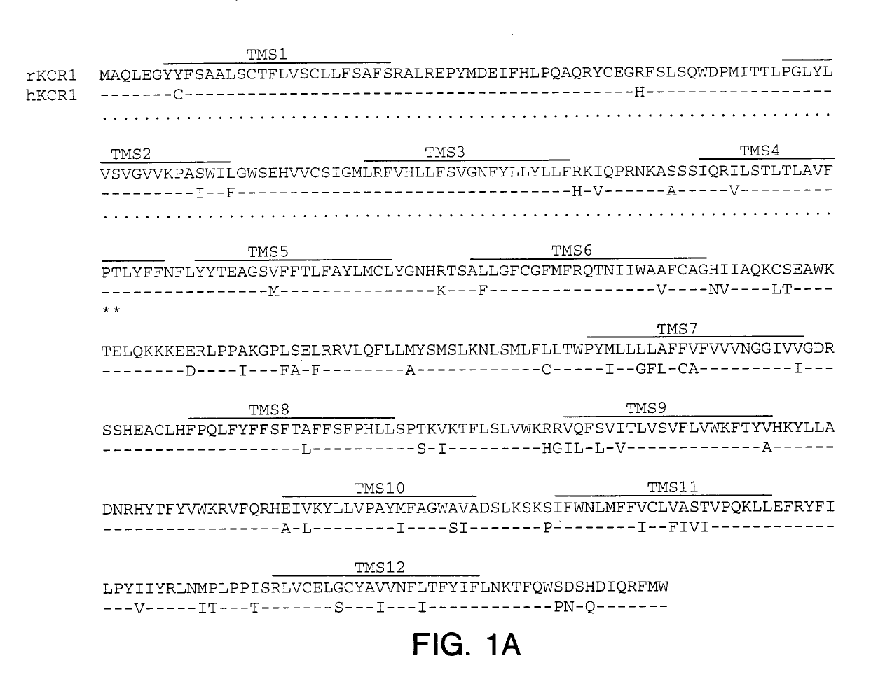

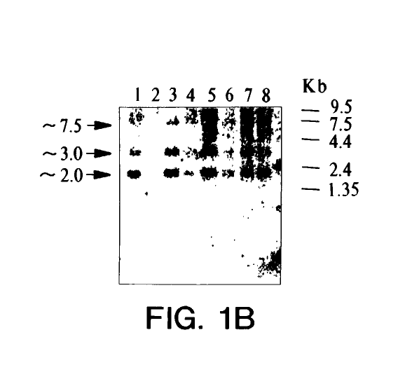

The human expressed sequence tag (EST) database (dbEST, National Center for Biotechnology Information) was queried with the nucleotide sequence of rat KCR1 (GenBank accession number U78090). This search resulted in the identification of a human EST containing cDNA sequence highly identical to rat KCR1. The corresponding I.M.A.G.E. cDNA (clone #650823) was purchased from Research Genetics of Huntsville, Ala., and its 2.6 kb insert was subcloned into the Xhol-EcoRl site of pBluescript™ for sequencing. The complete open reading frame (1422 bp) encodes a protein (designated hKCR1) with 86% amino acid identity to rat KCR1. Probes for Northern analysis were generated by PCR from the hKCR1 clone and directed against the first 422 nucleotides of the coding region. To examine tissue-specific expression (FIG. 1B), a human multiple tissue Northern blot was processed according to the manufacturer's instructions (Clontech of Palo Alto, Calif.).

### Plasmid cDNA Constructs and Transfection Strategy

The human ether-a-go-go related gene (HERG) cDNA was kindly provided by Dr. Mark Keating, University of Utah, and the coding region was subcloned into the mammalian expression vector PSI (Promega of Madison, Wis.) (Kupershmidt et al., (1998) J Biol Chem 273: 27231-27235). The rat KCR1 cDNA was provided by Dr. Haruhiro Higashida, Kanazawa University, Japan. This sequence was PCR amplified using primers to introduce unique Hind III and Mun I sites (5′ and 3′ respectively) and was subcloned into the Hind III/EcoR1 sites of pCGI (Johns et al., (1997) J Biol Chem 272: 31598-31603) for bicistronic expression of the protein with EGFP. MiRP1 cDNA was provided by Dr. Steve Goldstein, (Yale University) in vector pCl-neo (Promega of Madison, Wis.).

Chinese hamster ovary K1 (CHO-K1) cells were obtained from the American Type Culture Collection (Rockville, Md.) and cultured in Ham's F-12 media (Gibco-BRL of Grand Island, N.Y.) supplemented with 10% fetal bovine serum and 1% pen-strep in a humidified, 5% CO₂ incubator at 37° C. CHO-K1 cells were transiently transfected using the Lipofectamine transfection reagents and method (Gibco-BRL). When studying HERG alone or HERG+MiRP1, cells were cotransfected with pGFP-IRS (without KCR1). For experiments examining HERG+KCR1, or HERG+KCR1+MiRP1, GFP expression was obtained via the KCR1-containing pGFP-IRS plasmid. In all cases, cells displaying green fluorescence 48 to 72 hours after transfection were subjected to electrophysiologic analysis.

### Electrophysiology and Data Analysis

Potassium currents were recorded at room temperature (20-22° C.) using the whole-cell patch clamp technique. Electrodes resistances ranged from 1-2 M Ω when filled with a pipette intracellular solution containing: 110 mM KC1; 5 mM K₂ATP; 2 mM MgCl₂; 10 mM Hepes; and 5 mM K₄BAPTA, pH 7.2. The bath solution for all experiments contained: 145 mM NaCl; 4 mM KC1; 1.8 mM CaCl₂; 1.0 mM MgCl₂; 10 mM Hepes; and 10 mM glucose, pH 7.35. Dofetilide was provided by Pfizer Central Research of Groton, Conn., d-sotalol was provided by Bristol Meyers Squibb of Princeton, N.J., and quinidine was purchased from Sigma of St. Louis, Mo. Drug effects were recorded in cells following a pre-drug period where control data were obtained (during pulsing), and a 4 minute drug wash-in period throughout which the cell was held at −80 mV. The voltage clamp protocols used during drug exposure are described in the Brief Description of the Figures above and in the Laboratory Examples below, and the holding potential for all pulse protocols was −80 mV. Voltage clamp command pulses were generated, and patch clamp data were acquired using pCLAMP6 software (v6.0.4; Axon Instruments, Inc. of Foster City, Calif.). Currents were filtered at 5 kHz (−3 dB, 4-pole Bessel filter) and recorded using an AXOPATCH™ 200 integrating patch clamp amplifier (Axon Instruments, Inc. of Foster City, Calif.) with 80% series resistance compensation. Pooled data are presented as means and standard errors, and statistical comparisons were made by student t-test with p<0.05 considered significant.

### Laboratory Example 1

### Modulation of the Pharmacologic Properties of HERG by Human KCR1

A human KCR1 clone (hKCR1) was identified from an expressed sequence tag (EST) database (FIG. 1A) that exhibits 86% amino acid identity to rat KCR1. Expression of hKCR1 in human tissues was analyzed using Northern blot analysis (FIG. 1B). Two mRNA transcripts (approximately 25 and 3 kb respectively) were detected in all human tissues tested, including the heart. Both of these transcripts are large enough to encompass the complete human KCR1 coding region and could represent splice variants, or possibly independent transcripts from highly similar genes.

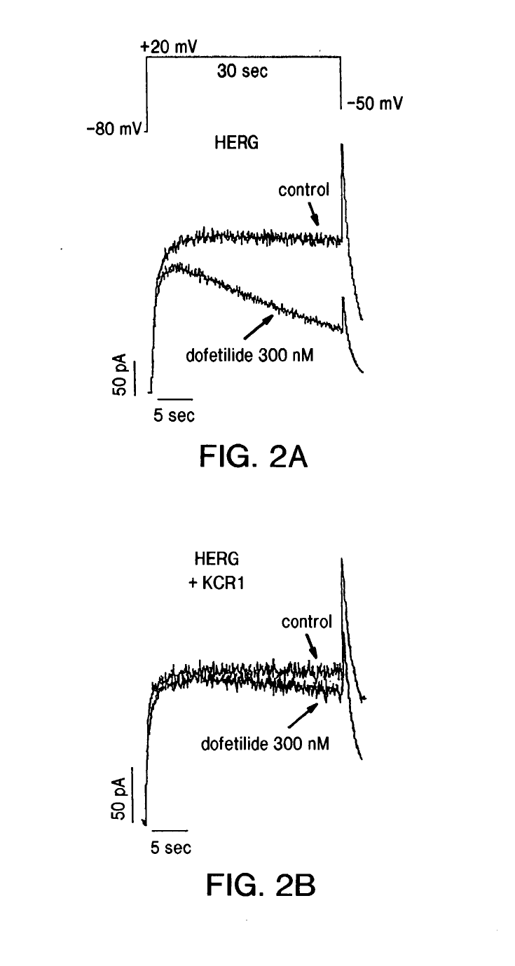

Then, whether KCR1 modulates the pharmacologic properties of HERG was tested. Dofetilide (sold under the trademark TIKOSYN® and commercially available from Pfizer Labs, Inc. of New York, N.Y.), a high-affinity blocker of I_{Kr} (Sanguinetti & Jurkiewicz, (1991) Am J Physiol 260: H393-H399) and HERG (Kiehn et al., (1996) Circulation 94: 2572-2579; Snyders & Chaudhary, (1996) Mol Pharmacol 49: 949-955), reduced HERG current in a time-dependent manner during a sustained depolarization to +30 m V (FIG. 2A). Despite this relatively high concentration (300 nM; therapeutic serum levels −10 nM), (Echt et al., (1995) J Cardiovasc Electr 6: 687-699) the blocking effect of dofetilide was markedly reduced by coexpression of KCRI (FIG. 2B).

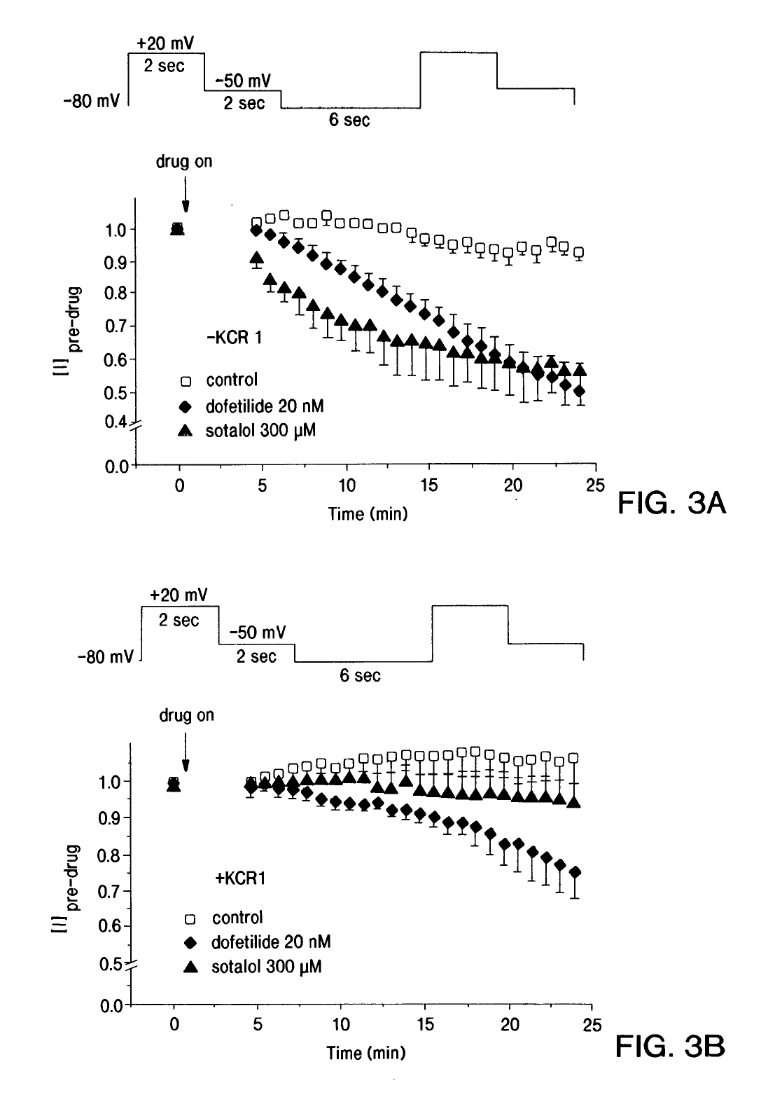

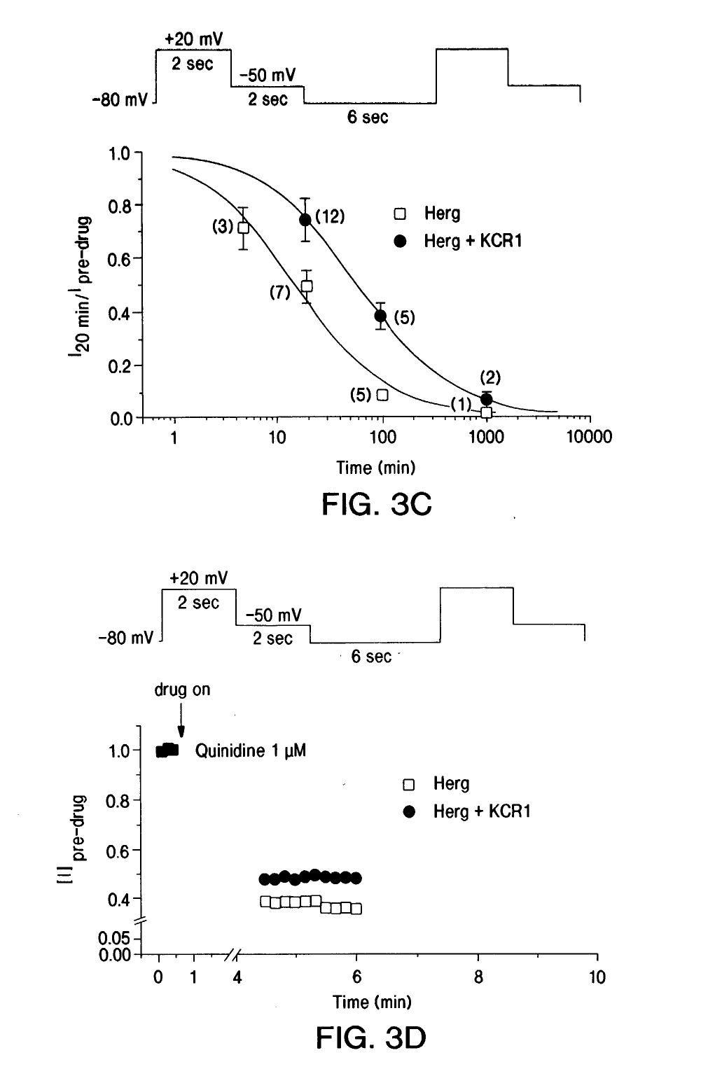

FIG. 3 examines the interaction between KCR1 and dofetilide when lower drug concentrations (20 nM) are utilized. In these conditions, HERG channel block develops slowly (over minutes) during continuous pulsing, as shown previously (Snyders & Chaudhary, (1996) Mol Pharmacol 49: 949-955; Spector et al., (1996) Circ Res 78: 499-503). After 20 minutes of exposure to 20 nM dofetilide, only 49±6% of the HERG current remained (FIG. 3A), while 74±8% of HERG+KCR1 current remained (FIG. 3B, p<0.05 vs. HERG alone). There was little or no time-dependent reduction in either HERG or HERG+KCR1 currents in drug-free conditions (FIGS. 3A and 3B, open squares). Similarly, it was found that hKCR1 cotransfection also reduced block by 20 nM dofetilide (remaining current with HERG+hKCR1 was 72±6%, n=5, p<0.05 vs. HERG alone). Exposure of HERG and HERG±KCR1 to a range of dofetilide concentrations revealed a rightward shift in the dose-response curve (FIG. 3C, HERG IC₅₀=15 nM, HERO+KCR1=59 nM).

### Laboratory Example 2

### Effect of KCR1 on HERG Block by d-Sotalol and Quinidine

The effect of KCR1 on HERG block by d-sotalol and quinidine, two compounds known to inhibit I_{Kr} (Sanguinetti & Jurkiewicz, (1990) J Gen Physiol 96: 195-215; Balser et al., (1991) Circ Res 69: 519-529) and provoke torsades de pointes (Roden, (1993) Am J Cardiol 72: 44B-49B), was also studied. Like dofetilide, block by d-sotalol developed over minutes (FIG. 3A), and KCR1 coexpression nearly eliminated the blocking effect (FIG. 3B). HERG tail current remaining after 20 minutes of d-sotalol exposure was 54±9% of the pre-drug control for HERG alone, but 95±6% for HERG+KCR1 (p <0.05 vs. HERG alone).

Quinidine (FIG. 3D), by contrast, produced rapid block, and reached an equilibrium level of current inhibition within the first few 15 test pulses. Despite these more rapid blocking characteristics, KCR1 reduced the extent of quinidine block; by the second pulse, the tail-current was 38±3% of the pre-drug control level for HERG alone, but 48+3% for HERG+KCR1 (p<0.05 versus HERG alone).

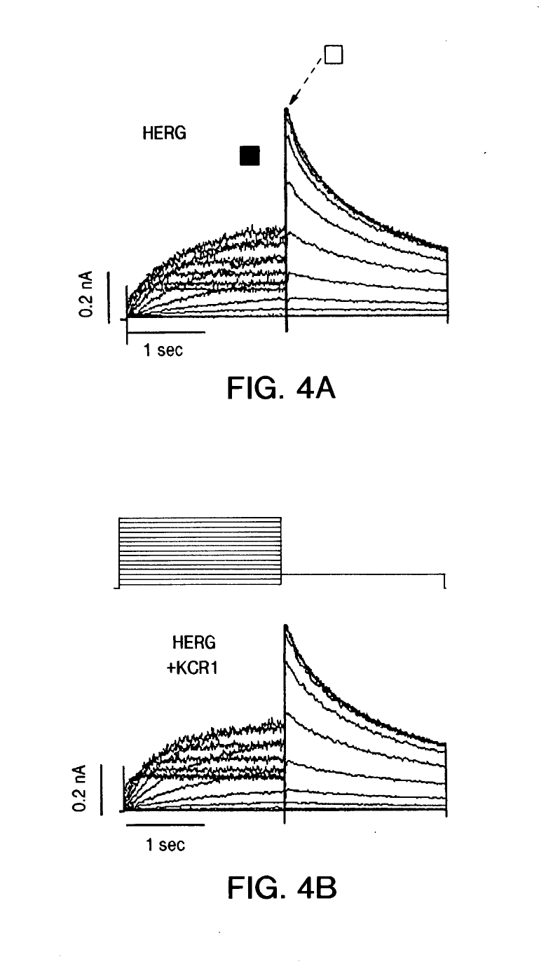

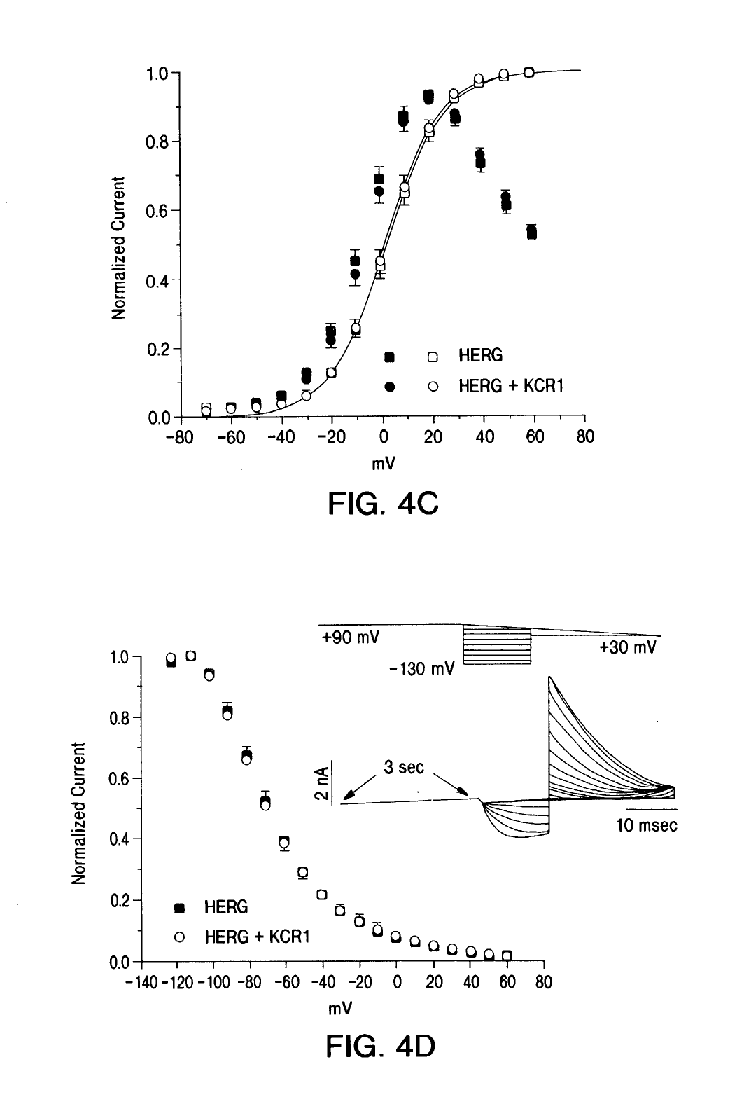

HERG block by most compounds develops when the channel opens (Kiehn et al., (1996) Circulation 94: 2572-2579; Snyders & Chaudhary, (1996) Mol Pharmacol 49: 949-955; Echt et al., (1995) J Cardiovasc Electr 6: 687-699), but might also be influenced by the inactivation gating transition (Ficker et al., (1998) Circ Res 82; Wang et al., (1997) FEBS Lett 417: 43-47; Lees-Miller et al., (2000) Mol Pharmacol 57: 367-374). It was therefore also assessed whether KCR1 alters the gating properties of HERG. FIG. 4 depicts families of currents recorded from cells expressing either HERG alone (FIG. 4A) or both HERG and KCR1 (FIG. 4B). The currents appear similar and, in both cases, the current-voltage relationship (FIG. 4C) exhibits the typical bell-shaped characteristic of HERG channels (Trudeau et al., (1995) Science 269: 92-95; Sanguinetti et al., (1995) Cell 81: 299-307).

### Laboratory Example 3

### KCR1 Effects on the Gating Properties of HERG Channels

KCR1 effects on the gating properties of HERG channels expressed in mammalian cells were assessed. FIG. 4C plots the peak tail current amplitude measured at a constant repolarized potential (−50 m V) following each depolarizing step to remove the confounding effects of HERG inactivation (Smith et al., (1996) Nature 379: 833-836; Spector et al., (1996) J Gen Physiol. 107: 611-619). The voltage-dependence of channel opening was not altered by KCRI expression; fitting a Boltzmann relationship to the data (solid line, FIG. 4C) yielded a half-maximal activation voltage of 2.7 mV for HERG alone, and 2.0 mV for HERG+KCRI.

The voltage-dependent distribution of channels between the open and inactivated states was also examined (FIG. 1D) by employing a 3-pulse clamp protocol (inset) (Smith et al., (1996) Nature 379: 833-836; Zou et al., (1998) J Physiol-Lond 509: 129-137). The instantaneous tail current amplitude was measured in the third step to +30 mV, and was plotted as a function of the preceding test potential. The data from cells expressing HERG alone and HERG+KCR1 superimpose, indicating that KCR1 has no effect on the voltage dependence of inactivation. These findings suggest that the inhibitory effects of KCR1 on HERG block do not result from indirect effects of KCR1 on HERG gating.

### Laboratory Example 4

### MiRP1 Interactions

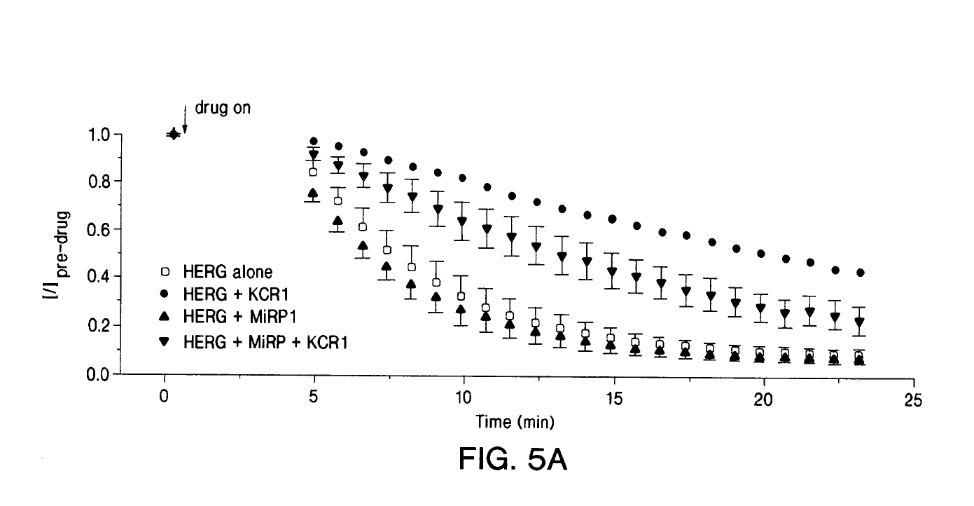

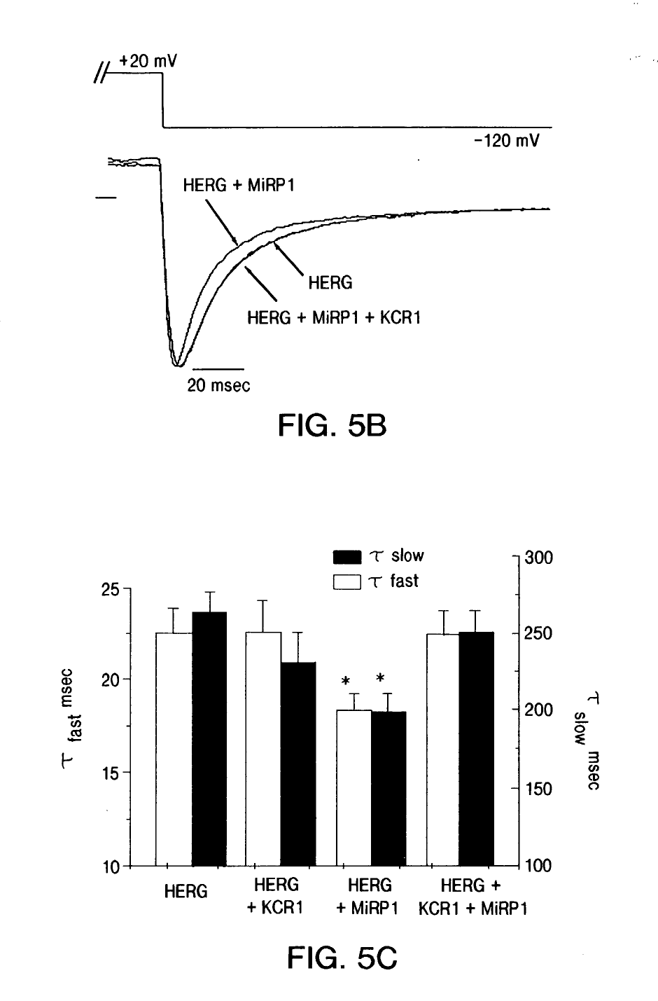

A prior study found that MiRP1 a small integral membrane peptide related to MinK, coassembles with HERG and could increase the sensitivity of HERG to drug block (Abbott et al., (1999) Cell 97: 175-187). Since the effect of KCR1 on HERG block is opposite to that of MiRP1, it was queried whether the two subunits, when coexpressed, would have antagonistic effects on dofetilide block. After 20 minutes, the currents generated from either HERG alone or HERG+MiRP1 were completely blocked by 100 nM dofetilide (FIG. 5A). In contrast, there was far less current blocked when HERG was coexpressed with KCR1 (62±5%), and expression of HERG with KCR1 and MiRP1 (HERG+KCR1+MiRP1) produced block that was intermediate in character (80±5%, FIG. 5A).

To confirm expression of MiRP1 and KCR1, the deactivating HERG current tail in each cell at −120 mV prior to drug application was examined. As shown previously (Abbott et al., (1999) Cell 97: 175-187), MiRP1 coassembly speeds deactivation of HERG (FIGS. 5B, 5C). Moreover, while KCR1 alone has no effect on the deactivation kinetics of HERG (FIG. 5C), it completely antagonizes the deactivation gating effects of MiRP1 (FIGS. 5B, 5C). Although it is not applicants' intention to be bound by any particular theory of operation, KCR1 might antagonize MiRP1 coassembly with HERG, or alternatively might allosterically inhibit the MiRP1 gating effect on HERG; in either case, this gating change suggests KCR1, when cotransfected, interacts with the HERG/MiRP1 complex.

### Laboratory Example 5

### KCR1 Polymorphisms

Given the evidence provided that KCR1 also modulates the blockade of HERG and I_{Kr} by drugs disclosed herein above, a database of DNA from acquired long QT patients collected at Vanderbilt University was examined. It was observed that the KCR1 polymorphism I447V is present at an allele frequency of 1.1%. This allele is significantly more common (7%, p<0.05 by Chi-Square analysis) in a control database of randomly selected individuals with ethnicities representing the Middle Tennessee area. Hence, it is envisioned that I447V is a risk-lowering allele in KCR1, which further provides that KCR1 is a screening target for gene sequence variations that raise or lower the risk of acquired long QT syndrome during drug therapy.

The genotyping primer pair that was used is as follows:

Forward: 5′-TTT CAA AGA TAT GCA ATT CTG-3′ (SEQ ID NO: 6)

Reverse: 5′-AAG TCC ATT TTT ACA GTT CA-3′ (SEQ ID NO: 7).

The amplification reactions were carried out in 50-μM volumes composed of 0.4 μM of each primer, 1× PCR buffer, 200 μM dNTPS. PCR reactions were performed under 95° C. for 10 minutes, then 95° C. 30 seconds, 54° C. 30 seconds, 72° C. seconds for 30 cycles, and 72° C. for additional 10 minutes. SSCP analysis was performed on 0.5× MDE gels that were electrophoresed overnight at 6W and subsequently stained with silver nitrate. Abnormal conformers were excised from the gel, eluted into sterile water, re-amplified and sequenced. The I447V variant was an A to G transition at nucleotide 1339 of KCR1 cDNA sequence.

### REFERENCES

The references listed below as well as all references cited in the specification are incorporated herein by reference to the extent that they supplement, explain, provide a background for or teach methodology, techniques and/or compositions employed herein. All cited patents and publications referred to in this application are herein expressly incorporated by reference. Also expressly incorporated herein by reference are the contents of all citations of GenBank accession numbers, LocusID, and other computer database listings.

Abbott, G. W., et al., Cell 97:175-87(1999)

Abdul M & Hoosein N (2001) Inhibition by anticonvulsants of prostate-specific antigen and interleukin-6 secretion by human prostate cancer cells. Anticancer Res 21:2045-2048.

Abruzzese R V, Godin D, Burcin M, Mehta V, French M, Li Y, O'Malley B W & Nordstrom J L (1999) Ligand-dependent regulation of plasmid-based transgene expression in vivo. Hum Gene Ther 10:1499-1507.

Ackerman & Clapham, (1997) N. Engl. J. Med. 336:11575-1586

Alekov A, Rahman M M, Mitrovic N, Lehmann-Horn F & Lerche H (2000) A sodium channel mutation causing epilepsy in man exhibits subtle defects in fast inactivation and activation in vitro. J Physiol 529 Pt 3:533-539.

Alekov A K, Rahman M M, Mitrovic N, Lehmann-Horn F & Lerche H (2001) Enhanced inactivation and acceleration of activation of the sodium channel associated with epilepsy in man. Eur J Neurosci 13:2171-2176.

Altschul S F, Gish W, Miller W, Myers E W & Lipman D J (1990) Basic local alignment search tool. J Mol Biol 215:403-410.

Antibodies A Laboratory Manual, E. Howell and D. Lane, Cold Spring Harbor Laboratory, 1988

Antzelevitch & Sicouri, (1994). J. Am. Col. Card. 23: 259-277

Ausubel (ed.) (1995) Short Protocols in Molecular Biology, 3rd ed. Wiley, New York, N.Y.

Balser et al., (1990). J. Gen. Physiol. 96: 835-863

Balser et al., (1991) Circ Res 69: 519-529

Barton G J (1998) Protein sequence alignment techniques. Acta Crystallogr D Biol Crystallogr 54:1139-1146.

Batzer M A, Carlton J E & Deininger P L (1991) Enhanced evolutionary PCR using oligonucleotides with inosine at the 3′-terminus. Nucleic Acids Res 19:5081.

Bauminger S & Wilchek M (1980) The use of carbodiimides in the preparation of immunizing conjugates. Methods Enzymol 70:151-159.

Beaucage et al., Tetrahedron Letters 22:1859-1862 (1981).

Berkow et al. (1997) The Merck Manual of Medical Information, Home ed. Merck Research Laboratories, Whitehouse Station, N.J.

Berkow R, Beers M H & Fletcher A J (1997) The Merck Manual of Medical Information, Home ed. Merck Research Laboratories, Whitehouse Station, N.J.

Blundell TL & Johnson L N (1976) Protein Crystallography. Academic Press, New York.

Bodanszky M (1993) Principles of Peptide Synthesis, 2nd rev. ed. Springer-Verlag, Berlin; New York.

Bruggemann et al., (1993). Nature 365: 445-448

Burcin M M, Schiedner G, Kochanek S, Tsai S Y & O'Malley B W (1999) Adenovirus-mediated regulable target gene expression in vivo. Proc Natl Acad Sci USA 96:355-360.

Caron J & Libersa C (1997) Adverse effects of class I antiarrhythmic drugs. Drug Saf 17:8-36.

Catterall W A, Morrow C S, Daly J W & Brown G B (1981) Binding of batrachotoxinin A 20-alpha-benzoate to a receptor site associated with sodium channels in synaptic nerve ending particles. J Biol Chem 256:8922-8927.

Chan D W (1996) Immunoassay Automation: A Practical Guide. Academic Press, San Diego.

Chang L T (1983) A method for attenuation correction in radionuclide computed tomography. IEEE Trans Nucl Sci NS-25:638-643.

Chen H, McCarty D M, Bruce A T & Suzuki K (1998) Gene transfer and expression in oligodendrocytes under the control of myelin basic protein transcriptional control region mediated by adeno-associated virus. Gene Ther 5:50-58.

Chen H, McCarty D M, Bruce A T & Suzuki K (1999) Oligodendrocyte-specific gene expression in mouse brain: use of a myelin-forming cell type-specific promoter in an adeno-associated virus. J Neurosci Res 55:504-513.

Cheng (1996) Hum Gene Ther 7:275-282

Chiang L W (1998) Detection of gene expression in single neurons by patch-clamp and single-cell reverse transcriptase polymerase chain reaction. J Chromatogr A 806:209-218.

Claes L, Del-Favero J, Ceulemans B, Lagae L, Van Broeckhoven C & De Jonghe P (2001) De novo mutations in the sodium-channel gene SCN1A cause severe myoclonic epilepsy of infancy. Am J Hum Genet 68:1327-1332.

Conner et al., Proc. Natl. Acad. Sci. U.S.A. 80:278 (1983)

Coulter D A (1997) Antiepileptic drug cellular mechanisms of action: where does lamotrigine fit in? J Child Neurol 12 Suppl 1:S2-9.

Curran et al., (1995) Cell. 80: 795-803

Curran et al., (1995) Cell. 81: 299-307

D'Arcangelo G, Paradiso K, Shepherd D, Brehm P, Halegoua S & Mandel G (1993) Neuronal growth factor regulation of two different sodium channel types through distinct signal transduction pathways. J Cell Biol 122:915-921.

Deisenhofer J, Epp O, Miki K, Huber R & Michel H (1984) X-ray structure analysis of a membrane protein complex. Electron density map at 3 A resolution and a model of the chromophores of the photosynthetic reaction center from Rhodopseudomonas viridis. J Mol Biol 180:385-398.

Deuschle U, Meyer W K & Thiesen H J (1995) Tetracycline-reversible silencing of eukaryotic promoters. Mol Cell Biol 15:1907-1914.

Dhillon H, Minter R, Topping D, Prima V, Moldawer L & Muzyczka N (1999) Long-term correction of obesity using centrally delivered rAAV encoding anorexigenic cytokines. Am Soc Gene Ther Abstr 2:45a.

Diss J K, Archer S N, Hirano J, Fraser S P & Djamgoz M B (2001) Expression profiles of voltage-gated Na(+) channel alpha-subunit genes in rat and human prostate cancer cell lines. Prostate 48:165-178.

Duch D S, Rehberg B & Vysotskaya TN (1998) Volatile anesthetics significantly suppress central and peripheral mammalian sodium channels. Toxicol Lett 100-101:255-263.

Dupere J R, Dale T J, Starkey S J & Xie X (1999) The anticonvulsant BW534U87 depresses epileptiform activity in rat hippocampal slices by an adenosine-dependent mechanism and through inhibition of voltage-gated Na⁺channels. Br J Pharmacol 128:1011-1020.

Ebadi MS (1998) CRC Desk Reference of Clinical Pharmacology. CRC Press, Boca Raton.

Echt et al., (1995) J Cardiovasc Electr 6: 687-699

Eglen R M, Hunter J C & Dray A (1999) Ions in the fire: recent ion-channel research and approaches to pain therapy. Trends Pharmacol Sci 20:337-342.

Elmer L W, O'Brien B J, Nutter T J & Angelides K J (1985) Physicochemical characterization of the alpha-peptide of the sodium channel from rat brain. Biochemistry 24:8128-8137.

Escayg A, Heils A, MacDonald B T, Haug K, Sander T & Meisler M H (2001) A novel SCN1A mutation associated with generalized epilepsy with febrile seizures plus—and prevalence of variants in patients with epilepsy. Am J Hum Genet 68:866-873.

Escayg A, MacDonald B T, Meisler M H, Baulac S, Huberfeld G, An-Gourfinkel I, Brice A, LeGuern E, Moulard B, Chaigne D, Buresi C & Malafosse A (2000) Mutations of SCN1A, encoding a neuronal sodium channel, in two families with GEFS+2. Nat Genet 24:343-345.

European Patent No. 0 439 095

Ficker et al., (1998) Circ Res 82

Folimer et al., (1992). Am. J. Physiol. 262: C75-C83

Forss-Petter S, Danielson P E, Catsicas S, Battenberg E, Price J, Nerenberg M & Sutcliffe J G (1990) Transgenic mice expressing beta-galactosidase in mature neurons under neuron-specific enolase promoter control. Neuron 5:187-197.

Freireich E J, Gehan E A, Rail D P, Schmidt L H & Skipper H E (1966) Quantitative comparison of toxicity of anticancer agents in mouse, rat, hamster, dog, monkey, and man. Cancer Chemother Rep 50:219-244.

Freshney, (1987) Culture of Animal Cells: A Manual of Basic Technique, 2nd ed. A. R. Liss, New York

Games D, Adams D, Alessandrini R, Barbour R, Berthelette P, Blackwell C, Carr T, Clemens J, Donaldson T, Gillespie F & et al. (1995) Alzheimer-type neuropathology in transgenic mice overexpressing V717F beta-amyloid precursor protein. Nature 373:523-527.

GenBank Accession Number AF257518

GenBank Accession Number U04270

GenBank Accession Number U42204

GenBank Accession Number U78090

GenBank Accession Number: BAA37096

GenBank Accession Number: SEG_AB00905S

GenBank Accession Number: Q9Y6J6

GenBank Accession Number: XM_{—}048634

Gerner E W, Hersh E M, Pennington M, Tsang T C, Harris D, Vasanwala F & Brailey J (2000) Heat-inducible vectors for use in gene therapy. Int J Hyperthermia 16:171-181.

Glover D M & Hames B D (1995) DNA Cloning: A Practical Approach, 2nd ed. IRL Press at Oxford University Press, Oxford/N.Y.

Gold M S (1999) Tetrodotoxin-resistant Na+currents and inflammatory hyperalgesia. Proc Natl Acad Sci USA 96:7645-7649.

Goldman C K, Rogers B E, Douglas J T, Sosnowski B A, Ying W, Siegal G P, Baird A, Campain J A & Curiel D T (1997) Targeted gene delivery to Kaposi's sarcoma cells via the fibroblast growth factor receptor. Cancer Res 57:1447-1451.

Goodman L S, Gilman A, Hardman J G, Gilman A G & Limbird L E (1996) Goodman & Gilman's the Pharmacological Basis of Therapeutics, 9th ed. McGraw-Hill Health Professions Division, New York.

Gossen M, Freundlieb S, Bender G, Muller G, Hillen W & Bujard H (1995) Transcriptional activation by tetracyclines in mammalian cells. Science 268:1766-1769.

Gribskov et al., (1986) Nucl. Acids. Res. 14: 6745

Habib et al. (1999) Human Gene Therapy 12:2019-2034

Henikoff & Henikoff, (1989) Proc Natl Acad Sci U.S.A. 89:10915

Henikoff J G, Pietrokovski S, McCallum C M & Henikoff S (2000) Blocks-based methods for detecting protein homology. Electrophoresis 21:1700-1706.

Henikoff S & Henikoff J G (1992) Amino acid substitution matrices from protein blocks. Proc Natl Acad Sci U S A 89:10915-10919.

Hickenbottom S L & Grotta J (1998) Neuroprotective therapy. Semin Neurol 18:485-492.

Homola et al. (1999) Sensors and Actuators 54:3-15.

Homola J, Yee S & Gauglitz G (1999) Surface plasmon resnoance sensors: review. Sensors and Actuators 54:3-15.

Hoppe, U. C., et al., Proc Natl Acad Sci USA. 98:5335-40 (2001).

Hoshi et al., (1998) J Biol Chem 273: 23080-23085

http://www.ihc.com/researchIlongqt.html

Huang C C, Novak W R, Babbitt P C, Jewett A I, Ferrin T E & Klein T E (2000) Integrated tools for structural and sequence alignment and analysis. Pac Symp Biocomput:230-241.

Huang et al. (2000) Pac Symp Biocomput:230-241.

Hub et al., (1980) Angew. Chem. Int. Ed. Engl. 19: 938

Hutchens & Yip (1993) Rapid Commun Mass Spectrom 7:576-580.

Ikenaka K & Kagawa T (1995) Transgenic systems in studying myelin gene expression. Dev Neurosci 17:127-136.

Ishikawa E (1999) Ultrasensitive and rapid enzyme immunoassay. Elsevier, Amsterdam/New York.

Isom L L, De Jongh K S, Patton D E, Reber B F, Offord J, Charbonneau H, Walsh K, Goldin A L & Catterall W A (1992) Primary structure and functional expression of the beta 1 subunit of the rat brain sodium channel. Science 256:839-841

January & Riddle, (1989). Circ. Res. 64: 977-990

Jellett J F, Marks L J, Stewart J E, Dorey M L, Watson-Wright W & Lawrence J F (1992) Paralytic shellfish poison (saxitoxin family) bioassays: automated endpoint determination and standardization of the in vitro tissue culture bioassay, and comparison with the standard mouse bioassay. Toxicon 30:1143-1156.

Jervell & Lange-Nielsen, (1957). Am. Heart J 54: 59-78

Ji H L, Fuller C M & Benos D J (1999) Peptide inhibition of constitutively activated epithelial Na(+) channels expressed in Xenopus oocytes. J Biol Chem 274:37693-37704.

Johns et al., (1997) J Biol Chem 272: 31598-31603

Kannel et al., (1987). Am. Heart J. 113: 799-804

Karlin S & Altschul S F (1993) Applications and statistics for multiple high-scoring segments in molecular sequences. Proc Natl Acad Sci USA 90:5873-5877.

Katzung B G (2001) Basic & Clinical Pharmacology, 8th ed. Lange Medical Books/McGraw-Hill Medical Pub. Division, New York.

Keating & Sanguinetti, (1996) Science 272: 681-685

Kiehn et al., (1996) Circulation 94: 2572-2579

Klein R L, Meyer E M, Peel A L, Zolotukhin S, Meyers C, Muzyczka N & King M A (1998) Neuron-specific transduction in the rat septohippocampal or nigrostriatal pathway by recombinant adeno-associated virus vectors. Exp Neurol 150:183-194.

Kogure K, Tamplin M L, Simidu U & Colwell R R (1988) A tissue culture assay for tetrodotoxin, saxitoxin and related toxins. Toxicon 26:191-197.

Kukuljan et al., (1991) J. Membrane Biol. 119: 187

Kupershmidt et al., (1998) J Biol Chem 273: 27231-27235

Kyte & Doolittle, (1982), J. Mol. Biol. 157:105-132

Landgren et. al., Science 241:1007,1988

Landgren et. al., Science 242:229-237 (1988)).

Law B (1996) Immunoassay: A Practical Guide. Taylor & Francis, London/Bristol, Pa.

Lees-Miller et al., (2000) Mol Pharmacol 57: 367-374

Lehmann-Horn F & Jurkat-Roft K (1999) Voltage-gated ion channels and hereditary disease. Physiol Rev 79:1317-1372.

Liddell E & Weeks I (1995) Antibody Technology. Bios Scientific Publishers, Oxford, United Kingdom.

Liedberg B, Nylander C & Lundstrom I (1983) Surface plasmon resonance for gas detection and biosensing. Sensors and Actuators 4:299-304.

Liposometechnology 2nd ed. Vol. I Liposome preparation and related techniques, (Gregoriadis, ed.) CRC Press, Boca Raton, Fla., 1993

Lopez et al., (1982) Biochim. Biophys. Acta 693: 437

Maalouf G J, Xu W, Smith T F & Mohr S C (1998) Homology model for the ligand-binding domain of the human estrogen receptor. J Biomol Struct Dyn 15:841-851.

Madio D P, van Gelderen P, DesPres D, Olson A W, de Zwart J A, Fawcett T W, Holbrook N J, Mandel M & Moonen C T (1998) On the feasibility of MRI-guided focused ultrasound for local induction of gene expression. J Magn Reson Imaging 8:101-104.

Magde D, Elsen E & Webb W (1972) Thermodynamic fluctuations in a reacting system: measurement by fluorescence correlation spectroscopy. Physical Review Letters 29:705-708.

Maiti S, Haupts U & Webb W W (1997) Fluorescence correlation spectroscopy: diagnostics for sparse molecules. Proc Natl Acad Sci USA 94:11753-11757.

Mak P, McDonnell D P, Weigel N L, Schrader W T & O'Malley B W (1989) Expression of functional chicken oviduct progesterone receptors in yeast (Saccharomyces cerevisiae). J Biol Chem 264:21613-21618.

Mandel R J, Rendahl K G, Spratt S K, Snyder R O, Cohen L K & Leff S E (1998) Characterization of intrastriatal recombinant adeno-associated virus-mediated gene transfer of human tyrosine hydroxylase and human GTP-cyclohydrolase I in a rat model of Parkinson's disease. J Neurosci 18:4271-4284.

Maniatis et. al. in Molecular Cloning: A Laboratory Manual, Cold Spring Harbor, N.Y., p 280-281 (1982)

Manson M M (1992) Immunochemical Protocols. Humana Press, Totowa, N.J. .

Masseyeff R F, Albert W H W & Staines N (1993) Methods of Immunological analysis. VCH Verlagsgesellschaft/VCH Publishers, Weinheim, Federal Republic of Germany/New York.

McCleskey E W & Gold M S (1999) Ion channels of nociception. Annu Rev Physiol 61:835-856.

McPherson (1982) The Preparation and Analysis of Protein Crystals. John Wiley, New York.

Miller, (1996) Nature 379: 767

Mitchell P H & Habermann B (1999) Rethinking physiologic stability: touch and intracranial pressure. Biol Res Nurs 1:12-19.

Morgan K, Stevens E B, Shah B, Cox P J, Dixon A K, Lee K, Pinnock R D, Hughes J, Richardson P J, Mizuguchi K & Jackson A P (2000) beta 3: an additional auxiliary subunit of the voltage-sensitive sodium channel that modulates channel gating with distinct kinetics. Proc Natl Acad Sci USA 97:2308-2313.

Moss et al., (1991) Circulation 84:1136-1144

Nabel (1997), Current Protocols in Human Genetics. John Wiley & Sons, New York, Vol. on CD-ROM

Nabel G (1997) Vectors for Gene Therapy. In: Current Protocols in Human Genetics. John Wiley & Sons, New York.

Needleman S B & Wunsch C D (1970) A general method applicable to the search for similarities in the amino acid sequence of two proteins. J Mol Biol 48:443-453.

Neri D, Carnemolla B, Nissim A, Leprini A, Querze G, Balza E, Pini A, Tarli L, Halin C, Neri P, Zardi L & Winter G (1997) Targeting by affinity-matured recombinant antibody fragments of an angiogenesis associated fibronectin isoform. Nat Biotechnol 15:1271-1275.

Niwa H, Yamamura K & Miyazaki J (1991) Efficient selection for high-expression transfectants with a novel eukaryotic vector. Gene 108:193-199.

No D, Yao T P & Evans R M (1996) Ecdysone-inducible gene expression in mammalian cells and transgenic mice. Proc Natl Acad Sci USA 93:3346-3351.

Ohtsuka E, Matsuki S, Ikehara M, Takahashi Y & Matsubara K (1985) An alternative approach to deoxyoligonucleotides as hybridization probes by insertion of deoxyinosine at ambiguous codon positions. J Biol Chem 260:2605-2608.

Oiki S, Madison V & Montal M (1990) Bundles of amphipathic transmembrane alpha-helices as a structural motif for ion-conducting channel proteins: studies on sodium channels and acetylcholine receptors. Proteins 8:226-236.

Oxender D L & Fox C F (1987) Protein Engineering. Liss, New York.

Park J W, Hong K, Kirpotin D B, Papahadjopoulos D & Benz C C (1997) Immunoliposomes for cancer treatment. Adv Pharmacol 40:399-435.

Pasqualini R, Koivunen E & Ruoslahti E (1997) Alpha v integrins as receptors for tumor targeting by circulating ligands. Nat Biotechnol 15:542-546.

PCR. A Practical Approach, ILR Press, Eds. McPherson et al. (1992)

Pearson W R & Lipman D J (1988) Improved tools for biological sequence comparison. Proc Natl Acad Sci USA 85:2444-2448.

Peel A L, Zolotukhin S, Schrimsher G W, Muzyczka N & Reier P J (1997) Efficient transduction of green fluorescent protein in spinal cord neurons using adeno-associated virus vectors containing cell type-specific promoters. Gene Ther 4:16-24.

Porreca F, Lai J, Bian D, Wegert S, Ossipov M H, Eglen R M, Kassotakis L, Novakovic S, Rabert D K, Sangameswaran L & Hunter J C (1999) A comparison of the potential role of the tetrodotoxin-insensitive sodium channels, PN3/SNS and NaN/SNS2, in rat models of chronic pain. Proc Natl Acad Sci USA 96:7640-7644.

Reith M E (1990) [14C]guanidinium ion influx into Na+channel preparations from mouse cerebral cortex. Eur J Pharmacol 188:33-41.

Remington J P, Osol A, Anderson J T, Hoover J E & Skolaut M W (1975) Remington's Pharmaceutical Sciences, 15th ed. Mack Pub. Co., Easton, Pa.

Rettig et al., (1994) Nature 369: 289-294

Roden, (1988). Arrhythmogenic Potential of Class III Antiarrhythmic Agents: Comparison with Class I Agents. in Control of Cardiac Arrhythmias by Lengthening Repolarization, Singh (ed.). Mt. Kisco, N.Y., Futura Publishing Co., pp. 559-576

Roden, (1993) Am J Cardiol 72: 44B-49B

Roden, (1998) Pacing Clin Electrophysiol 21: 1029-1034

Romano, (1965) Lancet 1658-659

Rossolini G M, Cresti S, Ingianni A, Cattani P, Riccio M L & Satta G (1994) Use of deoxyinosine-containing primers vs degenerate primers for polymerase chain reaction based on ambiguous sequence information. Mol Cell Probes 8:91-98.

Sabirov R Z, Azimov R R, Ando-Akatsuka Y, Miyoshi T & Okada Y (1999) Na(+) sensitivity of ROMK1 K(+) channel: role of the Na(+)/H(+) antiporter. J Membr Biol 172:67-76.

Saiki et al., Bio/Technology 3:1008-1012 (1985)

Sakmann & Neher, (1983) Single Channels Recordings, Plenum Press, New York, N.Y.

Sakmann & Neker, (1984) Ann. Rev. Physiol. 46: 455

Sambrook et al. eds (1989) Molecular Cloning: A Laboratory Manual. Cold Spring Harbor Laboratory Press, Cold Spring Harbor.

Sanguinetti & Jurkiewicz, (1990) J Gen Physiol 96: 195-215

Sanguinetti & Jurkiewicz, (1991) Am J Physiol 260: H393-H399

Sanguinetti et al., (1995) Cell 81: 299-307

Saqi et al. (1999) Bioinformatics 15:521-522.

Saqi M A, Wild D L & Hartshorn M J (1999) Protein analyst—a distributed object environment for protein sequence and structure analysis. Bioinformatics 15:521-522.

Schneider & Eberle (1993) Peptides, 1992: Proceedings of the Twenty-Second European Peptide Symposium, Sep. 13-19, 1992, Interlaken, Switzerland. Escom, Leiden; Bodanszky (1993) Principles of Peptide Synthesis, 2^{nd} rev. ed. Springer-Verlag, Berlin; New York

Schröder E & Lübke K (1965) The Peptides. Academic Press, New York.

Schwartz et al., (1975) Am. Heart J. 109: 378-390

Schwartz et al., (1994). The long QT Syndrome. in Cardiac Electrophysiology: From Cell to Bedside, (Zipes & Jalife, eds.), W. B. Sanders Company, pp.788-811

Schwartz et al., eds., (1979), Atlas of Protein Sequence and Structure, National Biomedical Research Foundation, pp. 357-358

Sesti, F., et al., Proc Natl Acad Sci USA. 97:10613-8 (2000)

Shibasaki, (1987). J. Physiol. 387: 227-250

Silhavy T J, Berman M L, Enquist L W & Cold Spring Harbor Laboratory. (1984) Experiments with Gene Fusions. Cold Spring Harbor Laboratory, Cold Spring Harbor, N.Y.

Sindrup S H & Jensen T S (2000) Pharmacologic treatment of pain in polyneuropathy. Neurology 55:915-920.

Smith et al., (1981) Adv. Appl. Math. 2:482

Smith et al., (1996) Nature 379: 33

Smith et al., (1996) Nature 379: 833-836

Smith M R, Smith R D, Plummer N W, Meisler M H & Goldin A L (1998) Functional analysis of the mouse Scn8a sodium channel. J Neurosci 18:6093-6102.

Smith R D & Goldin A L (1998) Functional analysis of the rat I sodium channel in Xenopus oocytes. J Neurosci 18:811-820.

Smith T F & Waterman M (1981) Comparison of Biosequences. Adv Appl Math 2:482-489.

Snyders & Chaudhary, (1996) Mol Pharmacol 49: 949-955

Spector et al., (1996) Circ Res 78: 499-503

Spector et al., (1996) J Gen Physiol 107: 611-619

Speight T M, Holford N H G & Avery G S (1997) Avery's Drug Treatment: A Guide to the Properties, Choice, Therapeutic Use and Economic Value of Drugs in Disease Management, 4th ed. Adis International, Auckland/Philadelphia.

Splawski et al., (1997) Nat Genet 17: 338-340

Squire I B, Lees K R, Pryse-Phillips W, Kertesz A & Bamford J (1995) Efficacy and tolerability of lifarizine in acute ischemic stroke. A pilot study. Lifarizine Study Group. Ann N Y Acad Sci 765:317-318.

Stoll J & Galdzicki Z (1996) Reduced expression of voltage-gated sodium channels in neurons cultured from trisomy 16 mouse hippocampus. Int J Dev Neurosci 14:749-760.

Stuhmer et al., (1989) EMBO J. 8(11): 3235-3244

Stuhmer W, Methfessel C, Sakmann B, Noda M & Numa S (1987) Patch clamp characterization of sodium channels expressed from rat brain cDNA. Eur Biophys J 14:131-138.

Surawicz, (1989). J. Am. Coll. Cardiol. 14: 172-184

Taglialatela M, Toro L & Stefani E (1992) Novel voltage clamp to record small, fast currents from ion channels expressed in Xenopus oocytes. Biophys J 61:78-82.

Teschemacher et al., (1999) Br J Pharmacol 128: 479-485

Tijssen (1993) Laboratory Techniques in Biochemistry and Molecular Biology—Hybridization with Nucleic Acid Probes. Elsevier, New York.

Titus et al., (1997) J. Neurosci. 17: 875-881

Trudeau et al., (1995) Science 269: 92-95

U.S. Pat. No. 4,683,195

U.S. Pat. No. 4,683,202

U.S. Pat. No. 4,965,188

U.S. Pat. No. 4,196,265

U.S. Pat. No. 4,455,842

U.S. Pat. No. 4,458,066

U.S. Pat. No. 4,551,482

U.S. Pat. No. 4,554,101

U.S. Pat. No. 4,737,323

U.S. Pat. No. 4,946,778

U.S. Pat. No. 5,008,050

U.S. Pat. No. 5,091,513

U.S. Pat. No. 5,132,405

U.S. Pat. No. 5,223,409

U.S. Pat. No. 5,234,933

U.S. Pat. No. 5,252,348

U.S. Pat. No. 5,260,203

U.S. Pat. No. 5,264,563

U.S. Pat. No. 5,326,902

U.S. Pat. No. 5,490,840

U.S. Pat. No. 5,498,538

U.S. Pat. No. 5,499,971

U.S. Pat. No. 5,499,971

U.S. Pat. No. 5,510,103

U.S. Pat. No. 5,574,172

U.S. Pat. No. 5,580,722

U.S. Pat. No. 5,627,158

U.S. Pat. No. 5,629,145

U.S. Pat. No. 5,650,489

U.S. Pat. No. 5,651,991

U.S. Pat. No. 5,667,988

U.S. Pat. No. 5,677,427

U.S. Pat. No. 5,688,931

U.S. Pat. No. 5,690,954

U.S. Pat. No. 5,702,892

U.S. Pat. No. 5,707,798

U.S. Pat. No. 5,714,166

U.S. Pat. No. 5,734,033

U.S. Pat. No. 5,738,996

U.S. Pat. No. 5,747,334

U.S. Pat. No. 5,756,291

U.S. Pat. No. 5,762,909

U.S. Pat. No. 5,762,918

U.S. Pat. No. 5,776,859

U.S. Pat. No. 5,780,225

U.S. Pat. No. 5,780,242.

U.S. Pat. No. 5,786,387

U.S. Pat. No. 5,824,483

U.S. Pat. No. 5,834,228

U.S. Pat. No. 5,840,479

U.S. Pat. No. 5,846,720

U.S. Pat. No. 5,855,900

U.S. Pat. No. 5,858,410

U.S. Pat. No. 5,858,670

U.S. Pat. No. 5,871,778

U.S. Pat. No. 5,872,011

U.S. Pat. No. 5,892,019

U.S. Pat. No. 5,912,132

U.S. Pat. No. 5,922,254

U.S. Pat. No. 5,922,356

U.S. Pat. No. 5,922,545

U.S. Pat. No. 5,928,944

U.S. Pat. No. 5,948,635

U.S. Pat. No. 5,962,667

U.S. Pat. No. 5,985,279

U.S. Pat. No. 5,994,392

U.S. Pat. No. 6,001,088

U.S. Pat. No. 6,041,253

U.S. Pat. No. 6,054,561

U.S. Pat. No. 6,057,098

U.S. Pat. No. 6,068,829

U.S. Pat. No. 6,071,890

U.S. Pat. No. 6,106,866

U.S. Pat. No. 6,107,059

U.S. Pat. No. 6,127,339

U.S. Pat. No. 6,140,123

U.S. Pat. No. 6,156,511

U.S. Pat. No. 6,165,440

U.S. Pat. No. 6,168,912

U.S. Pat. No. 6,174,690

U.S. Pat. No. 6,176,089

U.S. Pat. No. 6,177,088

U.S. Pat. No. 6,180,082

U.S. Pat. No. 6,180,348

U.S. Pat. No. 6,207,195

U.S. Pat. No. 6,214,553

U.S. Pat. No. 6,232,287

U.S. Pat. No. 6,234,990

U.S. Pat. No. 6,261,831

Vekris A, Maurange C, Moonen C, Mazurier F, De Verneuil H, Canioni P & Voisin P (2000) Control of transgene expression using local hyperthermia in combination with a heat-sensitive promoter. J Gene Med 2:89-96.

Vemuri et al.; (1995) Pharm. Acta Helvetiae 70: 95

Vincent et al., (1992) N. Engl. J. Med. 327: 846-852

Walker M R & Rapley R (1993) Molecular and Antibody Probes in Diagnosis. Wiley, Chichester, N.Y.

Wallace R H, Wang D W, Singh R, Scheffer I E, George A L, Jr., Phillips H A, Saar K, Reis A, Johnson E W, Sutherland G R, Berkovic S F & Mulley J C (1998) Febrile seizures and generalized epilepsy associated with a mutation in the Na+-channel beta1 subunit gene SCN1B. Nat Genet 19:366-370.

Wang et al., (1996) Nat Genet 12: 17-23

Wang et al., (1997) FEBS Lett 417: 43-47

Ward, (1964) J. Ir. Med. Assoc. 54: 103-106

Warmke & Ganetzky, (1994) Proc Nat'l Acad Sci USA 91: 3438-3442

Watwe et al., (1995) Curr. Sci. 68: 715

Waxman S G, Cummins T R, Dib-Hajj S, Fjell J & Black J A (1999b) Sodium channels, excitability of primary sensory neurons, and the molecular basis of pain. Muscle Nerve 22:1177-1187.

Waxman S G, Cummins T R, Dib-Hajj SD & Black J A (2000) Voltage-gated sodium channels and the molecular pathogenesis of pain: a review. J Rehabil Res Dev 37:517-528.

Waxman S G, Dib-Hajj S, Cummins T R & Black J A (1999a) Sodium channels and pain. Proc Natl Acad Sci USA 96:7635-7639.

Weichselbaum R R, Hallahan D, Fuks Z & Kufe D (1994) Radiation induction of immediate early genes: effectors of the radiation-stress response. Int J Radiat Oncol Biol Phys 30:229-234.

Weichselbaum R R, Hallahan D E, Sukhatme V, Dritschilo A, Sherman M L & Kufe D W (1991) Biological consequences of gene regulation after ionizing radiation exposure. J Natl Cancer Inst 83:480-484.

Weinshenker et al., (1999) J Neurosci 19: 9831-9840

Weiss M S, Wacker T, Weckesser J, Welte W & Schulz G E (1990) The three-dimensional structure of porin from Rhodobacter capsulatus at 3 A resolution. FEBS Lett 267:268-272.

Wetmur & Davidson, (1968) J. Mol. Biol. 31: 349-70

Willich et al., (1987). Am. J. Cardiol. 60: 801-806

WO 93/25521

WO 99/26966

Worrall T A, Cotter R J & Woods A S (1998) Purification of contaminated peptides and proteins on synthetic membrane surfaces for matrix-assisted laser desorption/ionization mass spectrometry. Anal Chem 70:750-756.

Wyckoff H W, Hirs C H W & Timasheff S N (1985) Diffraction Methods for Biological Macromolecules. Academic Press, Orlando, Fla.

Yamashita M, Ikemoto Y, Nielsen M & Yano T (1999) Effects of isoflurane and hexafluorodiethyl ether on human recombinant GABA(A) receptors expressed in Sf9 cells. Eur J Pharmacol 378:223-231

Yang et al., (1994). Circ. Res. 75: 870-878

Zipes, (1987). Am. J. Cardiol. 59: 26E-31E

Zou et al., (1998) J Physiol-Lond 509: 129-137

It will be understood that various details of the invention may be changed without departing from the scope of the invention. Furthermore, the foregoing description is for the purpose of illustration only, and not for the purpose of limitation-the invention being defined by the claims.

What is claimed is:

1. A method of identifying a compound that modulates a biological activity of a potassium channel, comprising:
(a) providing a structure comprising a potassium channel polypeptide and a KCR1 polypeptide;
(b) contacting the test compound with the structure;
(c) determining a biological activity of the potassium channel polypeptide in the presence of the test compound;
(d) comparing the biological activity of the potassium channel polypeptide in the presence of the test compound to the biological activity of the potassium channel polypeptide in an absence of the test compound, wherein a difference between the biological activity of the potassium channel in the absence of the test compound and the biological activity of the potassium channel polypeptide in the presence of test compound indicates modulation of a biological activity of the potassium channel.

2. The method of claim 1, wherein the structure comprises a cell.

3. The method of claim 2, wherein the cell is isolated from a subject.

4. The method of claim 1, wherein the structure comprises a lipid bilayer.

5. The method of claim 1, wherein the structure is a cell that has been transfected with a nucleic acid encoding an exogenous KCR1 polypeptide

6. The method of claim 1, wherein the structure is a cell that has been transfected with a nucleic acid encoding an exogenous potassium channel polypeptide.

7. The method of claim 1, wherein the potassium channel is HERG.

8. The method of claim 7, wherein the HERG potassium channel is comprises a polypeptide sequence as set forth in SEQ ID NO: 3.

9. The method of claim 8, wherein a nucleic acid encoding the HERG potassium channel is heterologous.

10. The method of claim 8, wherein a nucleic acid encoding the HERG potassium channel is polycistronic.

11. The method of claim 1, wherein the KCR1 polypeptide is encoded by a nucleic acid comprising SEQ ID NO: 1.

12. The method of claim 11, wherein the nucleic acid is heterologous.

13. The method of claim 11, wherein the nucleic acid is polycistronic.

14. The method of claim 1, wherein the determining comprises employing a patch clamp apparatus.

15. The method of claim 1, wherein the biological activity of a structure comprising a potassium channel polypeptide and a KCR1 polypeptide in the presence of a test compound is determined in the presence of an MiRP1 polypeptide.

16. The method of claim 1, wherein the structure further comprises a MiRP1 polypeptide.

17. The method of claim 16, wherein the MiRP1 polypeptide is encoded by a nucleic acid comprising SEQ ID NO: 4.

18. The method of claim 17, wherein the nucleic acid is heterologous.

19. The method of claim 17, wherein the nucleic acid is polycistronic.

20. A method of predicting a propensity of a candidate drug to induce a cardiac arrhythmia, comprising:
(a) providing a structure comprising a potassium channel and a KCR1 polypeptide;
(b) contacting a candidate drug with the structure;
(c) determining a biological activity of the potassium channel in the presence of the candidate drug; and
(d) comparing the biological activity of the potassium channel in the presence of a KCR1 polypeptide and in an absence of a candidate drug to a biological activity of the potassium channel in the presence of the candidate drug, wherein a biological activity of the potassium channel in the presence of a candidate drug that is less than a biological activity of the potassium channel in an absence of the candidate drug is indicative of a propensity of the drug to induce cardiac arrhythmia.

21. The method of claim 20, wherein the structure is selected from the group consisting of a cell and a lipid bilayer.

22. The method of claim 20, wherein the potassium channel is HERG.

23. The method of claim 22, wherein the HERG potassium channel comprises a polypeptide sequence as set forth in SEQ ID NO: 3.

24. The method of claim 23, wherein a nucleic acid encoding the HERG potassium channel is heterologous.

25. The method of claim 23, wherein a nucleic acid encoding the HERG potassium channel is polycistronic.

26. The method of claim 20, wherein the KCR1 polypeptide is encoded by a nucleic acid comprising SEQ ID NO: 1.

27. The method of claim 26, wherein the nucleic acid is heterologous.

28. The method of claim 26, wherein the nucleic acid is polycistronic.

29. The method of claim 30, wherein the determining comprises employing a patch clamp apparatus.

30. The method of claim 20, wherein the structure further comprises a MiRP1 polypeptide.

31. The method of claim 30, wherein the MiRP1 polypeptide is encoded by a nucleic acid comprising SEQ ID NO: 4.

32. The method of claim 31, wherein the nucleic acid is heterologous.

33. The method of claim 31, wherein the nucleic acid is polycistronic.

34. A method of identifying a candidate compound that modulates the biological activity of a complex comprising a HERG channel polypeptide and a KCR1 polypeptide, the method comprising:
(a) placing a cell comprising a HERG channel polypeptide and a KCR1 polypeptide into a bathing solution;
(b) determining an induced K⁺ current in the cell of step (a);
(c) adding a candidate drug to the bathing solution of step (a);
(d) determining an induced K⁺ current in the cell of step (c); and
(e) comparing the induced current of step (b) with the induced current of step (d), wherein the candidate compound modulates the biological activity of a complex comprising a HERG channel polypeptide and a KCR1 polypeptide if the current of step (d) is different from the current of step (b).

35. The method of claim 34, wherein the HERG channel polypeptide comprises a polypeptide sequence as set forth in SEQ ID NO: 3.

36. The method of claim 35, wherein a nucleic acid encoding the HERG potassium channel is heterologous.

37. The method of claim 35, wherein a nucleic acid encoding the HERG potassium channel is polycistronic.

38. The method of claim 45, wherein the KCR1 polypeptide is encoded by a nucleic acid comprising SEQ ID NO: 1.

39. The method of claim 38, wherein the nucleic acid is heterologous.

40. The method of claim 38, wherein the nucleic acid is polycistronic.

41. The method of claim 34, wherein the determining comprises employing a patch clamp apparatus.

42. The method of claim 34, wherein the cell further comprises a MiRP1 polypeptide.

43. The method of claim 42, wherein the MiRP1 polypeptide is encoded by a nucleic acid comprising SEQ ID NO: 4.

44. The method of claim 43, wherein the nucleic acid is heterologous.

45. The method of claim 43, wherein the nucleic acid is polycistronic.

46. The method of claim 34, wherein the cell is isolated from a subject.

47. The method of claim 34, further comprising transfecting the cell with a nucleic acid sequence encoding a HERG channel polypeptide and a nucleic acid sequence encoding a KCR1 polypeptide.

48. A modulator identified by the method of claim 34.

49. A method for identifying a candidate compound as a modulator of KCR1 expression, the method comprising:
(a) contacting a eukaryotic cell sample with a predetermined concentration of the candidate compound to be tested, the cell sample comprising at least one cell comprising a DNA construct comprising in 5′ to 3′ order (i) a modulatable transcriptional regulatory sequence of a KCR1-encoding gene, (ii) a promoter of the KCR1-encoding gene, and (iii) a reporter gene which expresses a polypeptide capable of producing a detectable signal coupled to and under the control of the promoter, under conditions such that the candidate compound if capable of acting as a transcriptional modulator of the gene encoding the protein of interest, causes a measurable detectable signal to be produced by the polypeptide expressed by the reporter gene;
(b) quantitatively determining the amount of the signal so produced; and
(c) comparing the amount so determined with the amount of produced signal detected in the absence of candidate compound being tested or upon contacting the cell sample with other compounds so as to thereby identify the candidate compound as a chemical which causes a change in the detectable signal produced by the polypeptide and which transcriptionally modulates expression of KCR1.

50. The method of claim 49, which comprises separately contacting each of a plurality of identical cell samples with different candidate compounds, each cell sample containing a predefined number of identical cells under conditions wherein said contacting is effected with a predetermined concentration of each different candidate compound to be tested.

51. A modulator identified by the method of claim 49.

52. A method for identifying a candidate compound as a modulator of KCR1 expression, the method comprising:
(a) contacting a eukaryotic cell sample with a predetermined concentration of the candidate compound to be tested, the cell sample comprising at least one cell comprising a DNA construct comprising in 5′ to 3′ order (i) a modulatable transcriptional regulatory sequence of a KCR1-encoding gene, (ii) a promoter of the KCR1-encoding gene, and (iii) a DNA sequence transcribable into mRNA coupled to and under the control of the promoter, under conditions such that the candidate compound if capable of acting as a transcriptional modulator of the KCR1-encoding gene, causes a measurable difference in the amount of mRNA transcribed from the DNA sequence;
(b) quantitatively determining the amount of the mRNA so produced; and
(c) comparing the amount so determined with the amount of mRNA detected in the absence of candidate compound being tested or upon contacting the cell sample with other compounds so as to thereby identify the candidate compound as a compound which causes a change in the detectable mRNA amount and which transcriptionally modulates expression of KCR1.

53. The method of claim 52, which comprises separately contacting each of a plurality of identical cell samples with different candidate compounds, each cell sample containing a predefined number of identical cells under conditions wherein said contacting is effected with a predetermined concentration of each different candidate compound to be tested.

54. A modulator identified by the method of claim 52.

55. A method for modulating potassium channel function in a subject, the method comprising:
(a) administering to the subject an effective amount of a substance that provides expression of a KCR1-encoding nucleic acid molecule in a cell or tissue where modulated potassium channel function is desired; and
(b) modulating potassium channel function in the subject through the administering of step (a).

56. The method of claim 55, wherein the subject is a mammal.

57. The method of claim 55, wherein the potassium channel function that is modulated in the subject comprises HERG function.

58. The method of claim 55, wherein the cell or tissue is a cardiac cell or tissue.

59. The method of claim 55, wherein the administering is selected for the group consisting of intravenous administration, intrasynovial administration, transdermal administration, intramuscular administration, subcutaneous administration and oral administration.

60. The method of claim 55, further comprising:
(a) providing a gene therapy construct comprising a nucleotide sequence encoding a KCR1 polypeptide; and
(b) administering the gene therapy construct to a subject, whereby the function of a potassium channel in the subject is modulated.

61. The method of claim 60, wherein the KCR1 polypeptide is encoded by a nucleic acid comprising SEQ ID NO: 1.

62. The method of claim 60, further comprising administering the gene therapy vector to a cardiac cell or tissue in the subject.

63. A method for modulating potassium channel function in a subject, the method comprising:
(a) preparing a composition comprising a modulator identified according to the method of claim 36, and a pharmaceutically acceptable carrier; and
(b) administering an effective dose of the pharmaceutical composition to a subject, whereby potassium channel activity is modulated in the subject.

64. The method of claim 63, wherein the subject is a mammal.

65. The method of claim 63, wherein the potassium channel activity that is modulated in the subject comprises HERG activity.

66. A method of screening for susceptibility to a drug-induced cardiac arrhythmia in a subject, the method comprising:
(a) obtaining a biological sample from the subject; and
(b) detecting a polymorphism of a KCR1 gene in the biological sample from the subject, the presence of the polymorphism indicating the susceptibility of the subject to a drug-induced cardiac arrhythmia.

67. The method of claim 66, wherein the biological sample comprises a nucleic acid sample.

68. The method of claim 67, wherein the polymorphism is an I447V polymorphism of the KCR1 gene.

69. The method of claim 68, wherein the polymorphism is detected by amplifying a target nucleic acid in the nucleic acid sample from the subject using an amplification technique.

70. The method of claim 69, wherein the polymorphism is detected by amplifying a target nucleic acid in the nucleic acid sample from the subject using an oligonucleotide pair, wherein a first oligonucleotide of the pair hybridizes to a first portion of the KCR1 gene, wherein the first portion includes the polymorphism of the KCR1 gene, and wherein the second of the oligonucleotide pair hybridizes to a second portion of the KCR1 gene that is adjacent to the first portion.

71. The method of claim 70, wherein the first and the second oligonucleotides each further comprise a detectable label, and wherein the label of the first oligonucleotide is distinguishable from the label of the second oligonucleotide.

72. The method of claim 71, wherein said label of said first oligonucleotide is a radiolabel, and wherein said label of said second oligonucleotide is a biotin label.

73. The method of claim 67, wherein the polymorphism is detected by sequencing a target nucleic acid in the nucleic acid sample from the subject.

74. The method of claim 73, wherein the sequencing comprises dideoxy sequencing.

75. The method of claim 67, wherein the step of detecting the polymorphism is detected by contacting a target nucleic acid in the nucleic acid sample from the subject with a reagent that detects the presence of the polymorphism and detecting the reagent.

76. The method of claim 75, wherein the reagent comprises an allele specific oligonucleotide.

77. The method of claim 66, wherein the subject is a human subject.

78. The method of claim 66, wherein the biological sample comprises a polypeptide sample

79. An oligonucleotide pair, wherein a first oligonucleotide of the pair hybridizes to a first portion of the KCR1 gene, wherein the first portion includes a polymorphism of the KCR1 gene, and wherein the second of the oligonucleotide pair hybridizes to a second portion of the KCR1 gene that is adjacent to the first portion.

80. The oligonucleotide pair of claim 79, wherein the polymorphism is an I447V polymorphism of the KCR1 gene.

81. The oligonucleotide pair of claim 79, wherein said first and said second oligonucleotides each further comprise a detectable label, and wherein said label of said first oligonucleotide is distinguishable from said label of said second oligonucleotide.

82. The oligonucleotide pair of claim 81, wherein said label of said first oligonucleotide is a radiolabel, and wherein said label of said second oligonucleotide is a biotin label.

83. A set of oligonucleotide primers comprising an anti-sense primer and a sense primer, wherein said oligonucleotide primer set is suitable for amplifying a portion of the KCR1 gene, wherein the portion includes a polymorphism of the KCR1 gene.

84. The oligonucleotide set of claim 83, wherein the polymorphism is an I447V polymorphism of the KCR1 gene.

85. A kit for detecting a polymorphism in a KCR1 gene, the kit comprising:
(a) a reagent for detecting the presence of a I447V polymorphism of the KCR1 gene in a biological sample from the subject; and
(b) a container for the reagent.

86. The kit of claim 85, wherein the polymorphism is an I447V polymorphism of the KCR1 gene.

87. The kit of claim 86, further comprising a reagent for amplifying a nucleic acid molecule containing an I447V polymorphism of the KCR1 gene.

88. The kit of claim 87, wherein the amplifying reagent comprises a polymerase enzyme suitable for use in a polymerase chain reaction and a pair of oligonucleotides.

89. The kit of claim 88, wherein a first oligonucleotide of the pair of oligonucleotides hybridizes to a first portion of the KCR1 gene, wherein the first portion includes the I447V polymorphism of the KCR1 gene, and wherein the second of the oligonucleotide pair hybridizes to a second portion of the KCR1 gene that is adjacent to the first portion.

90. The kit of claim 85, further comprising a reagent for extracting a nucleic acid sample from a biological sample obtained from a subject.

91. An assay kit for detecting the presence of a polymorphism of a KCR1 gene encoding a KCR1 polypeptide in a biological sample, the kit comprising a first container containing a first antibody capable of immunoreacting with a KCR1 subunit polypeptide encoding by a KCR1 gene comprising a polymorphism, wherein the first antibody is present in an amount sufficient to perform at least one assay.

92. The assay kit of claim 91, wherein the polymorphism is an I447V polymorphism of the KCR1 gene.

93. The assay kit of claim 91, further comprising a second container containing a second antibody that immunoreacts with the first antibody

94. The assay kit of claim 93, wherein the first antibody and the second antibody comprise monoclonal antibodies.

95. The assay kit of claim 93, wherein the first antibody is affixed to a solid support.

96. The assay kit of claim 93, wherein the first and second antibodies each comprise an indicator.

97. The assay kit of claim 96, wherein the indicator is a radioactive label or an enzyme.

98. An assay kit for detecting the presence, in a biological sample, of an antibody immunoreactive with a KCR1 polypeptide encoding by a KCR1 comprising a polymorphism, the kit comprising a first container containing a human KCR1 polypeptide encoded by a KCR1 gene comprising a polymorphism that immunoreacts with the antibody, with the polypeptide present in an amount sufficient to perform at least one assay.

99. The assay kit of claim 98, wherein the polymorphism is an I447V polymorphism of the KCR1 gene.
